SEQUENCE LISTING (45 sequences)

SEQ ID NO: 1 - protein, 290 aa, Homo sapiens
  REGION 1..290: note = PD-L1 protein
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 873 bp, Homo sapiens
  source 1..873: mol_type = unassigned DNA
  misc_feature 1..873: note = PD-L1 DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 3 - protein, 223 aa, Homo sapiens
  REGION 1..223: note = CTLA4 protein
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 4 - DNA, 672 bp, Homo sapiens
  source 1..672: mol_type = unassigned DNA
  misc_feature 1..672: note = CTLA4 DNA
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 5-6 - skipped (2 entries)

SEQ ID NO: 7 - protein, 323 aa, Homo sapiens
  REGION 1..323: note = CD47 protein
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 8 - DNA, 972 bp, Homo sapiens
  source 1..972: mol_type = unassigned DNA
  misc_feature 1..972: note = CD47 DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 9 - protein, 510 aa, Homo sapiens
  REGION 1..510: note = CD39 protein
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 10 - DNA, 1533 bp, Homo sapiens
  source 1..1533: mol_type = unassigned DNA
  misc_feature 1..1533: note = CD39 DNA
atggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtatag

SEQ ID NO: 11 - protein, 574 aa, Homo sapiens [sequence omitted]

SEQ ID NO: 12 - DNA, 1725 bp, Homo sapiens
  source 1..1725: mol_type = unassigned DNA
  misc_feature 1..1725: note = CD73 DNA
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 13 - protein, 178 aa, Homo sapiens
  REGION 1..178: note = IL-10 protein
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 14 - DNA, 537 bp, Homo sapiens
  source 1..537: mol_type = unassigned DNA
  misc_feature 1..537: note = IL-10 DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 15 - protein, 403 aa, Homo sapiens [sequence omitted]

SEQ ID NO: 16 - DNA, 1212 bp, Homo sapiens
  source 1..1212: mol_type = unassigned DNA
  misc_feature 1..1212: note = IDO1 DNA
atggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaa

SEQ ID NO: 17-20 - skipped (4 entries)

SEQ ID NO: 21 - DNA, 21 bp, synthetic construct
  source 1..21: mol_type = other DNA
  misc_feature 1..21: note = GAPDH forward primer
aggtcggtgtgaacggatttg

SEQ ID NO: 22 - DNA, 19 bp, synthetic construct
  source 1..19: mol_type = other DNA
  misc_feature 1..19: note = GAPDH reverse primer
ggggtcgttgatggcaaca

SEQ ID NO: 23 - DNA, 20 bp, synthetic construct
  source 1..20: mol_type = other DNA
  misc_feature 1..20: note = CTLA4 forward primer
agcagtagatacggggctct

SEQ ID NO: 24 - DNA, 20 bp, synthetic construct
  source 1..20: mol_type = other DNA
  misc_feature 1..20: note = CTLA4 reverse primer
tatcgggacacggttcagga

SEQ ID NO: 25 - DNA, 21 bp, synthetic construct
  source 1..21: mol_type = other DNA
  misc_feature 1..21: note = PD-L1 forward primer
gctccaaaggacttgtacgtg

SEQ ID NO: 26 - DNA, 21 bp, synthetic construct
  source 1..21: mol_type = other DNA
  misc_feature 1..21: note = PD-L1 reverse primer
tgatctgaagggcagcatttc

SEQ ID NO: 27 - DNA, 19 bp, synthetic construct
  source 1..19: mol_type = other DNA
  misc_structure 1..19: note = TGF-beta1 forward primer
ggccagatcctgtccaagc

SEQ ID NO: 28 - DNA, 21 bp, synthetic construct
  source 1..21: mol_type = other DNA
  misc_feature 1..21: note = TGF-beta1 reverse primer
gtgggtttccaccattagcac

SEQ ID NO: 29 - DNA, 22 bp, synthetic construct
  source 1..22: mol_type = other DNA
  misc_feature 1..22: note = Arg-1 forward primer
cattggcttgcgagacgtagac

SEQ ID NO: 30 - DNA, 22 bp, synthetic construct
  source 1..22: mol_type = other DNA
  misc_feature 1..22: note = Arg-1 reverse primer
gctgaaggtctcttccatcacc

SEQ ID NO: 31 - DNA, 22 bp, synthetic construct
  source 1..22: mol_type = other DNA
  misc_feature 1..22: note = iNOS forward primer
gagacagggaagtctgaagcac

SEQ ID NO: 32 - DNA, 22 bp, synthetic construct
  source 1..22: mol_type = other DNA
  misc_feature 1..22: note = iNOS reverse primer
ccagcagtagttgctcctcttc

SEQ ID NO: 33 - DNA, 22 bp, synthetic construct
  source 1..22: mol_type = other DNA
  misc_feature 1..22: note = TNF-alpha forward primer
ggtgcctatgtctcagcctctt

SEQ ID NO: 34 - DNA, 23 bp, synthetic construct
  source 1..23: mol_type = other DNA
  misc_feature 1..23: note = TNF-alpha reverse primer
gccatagaactgatgagagggag

SEQ ID NO: 35 - protein, 10 aa, synthetic construct
  REGION 1..10: note = BDC2.5 peptide
RTRPLWVRME

SEQ ID NO: 36 - protein, 9 aa, synthetic construct
  REGION 1..9: note = IGRP peptide
VYLKTNVFL

SEQ ID NO: 37 - DNA, 3030 bp, synthetic construct
  source 1..3030: mol_type = other DNA
  misc_feature 1..3030: note = synthetic mouse EF1a/IDO1/IL-10 construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggcactcagtaaaatatctcctacagaaggttctagaaggatccttgaagaccaccacatagatgaagatgtgggctttgctctaccacatccactggtggagctgcccgacgcatacagcccctgggtccttgtggctagaaatctgcctgtgctgattgagaacgggcagcttcgagaagaagttgaaaagctgcccacactgagcacggacggactgagaggacacaggttacagcgcctggcacacctggccctggggtacatcaccatggcgtatgtgtggaaccgaggggatgacgatgttcgaaaggtgctgccccgcaatattgctgttccctactgcgagctctcagagaagttgggcctgcctcctattctgtcttatgcagactgtgtcctggcaaactggaagaaaaaggaccccaatgggcccatgacatacgagaacatggacattctgttctcatttcctggtggggactgcgacaagggcttcttcctcgtctctctattggtggaaatcgcagcttctcctgcaatcaaagcaatccccactgtatccagtgcagtagagcgtcaagacctgaaagcattggaaaaggcactgcacgacatagctaccagtctggagaaagccaaggaaatttttaagaggatgcgtgactttgtggacccagacacgtttttccacgttctccgcatatatctgtctggctggaaatgcagctccaagctgccagaaggtctgctgtatgagggggtctgggacaccccaaaaatgttttcagggggcagtgcaggccagagcagcatcttccagagtcttgatgtccttctgggaataaaacacgaggctggcaaagaatctcctgcagaattcctccaggaaatgagagagtacatgcctccagcccaccggaacttccttttcttcttagagtcagctcccccagtccgtgagtttgtcatttcaagacacaatgaagacttgacgaaagcttataacgagtgtgtgaatggtctggtctctgtgagaaagttccacctcgcaatagtagatacttacattatgaaaccttcgaagaagaagcccactgatggcgacaagtcggaagagccctcaaatgtggaaagcagagggactgggggtacgaatcccatgactttcctaaggagtgtgaaagatacaaccgagaaagctcttctgagttggcctggaagcggagagggcaggggaagtcttctaacatgcggggacgtggaggaaaatcccggccccatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 38 - DNA, 4620 bp, synthetic construct
  source 1..4620: mol_type = other DNA
  misc_feature 1..4620: note = synthetic mouse EF1a/ CD39/CD73 construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggcagctggcagagggttggcgcgctttacggacctggaagaattggatgaagtcatcactgagaggaagaccaagaggcacaaactgagatcttaccaagattctaaggtgaagagattttgctccaaaaatattctgatcatccttggtttcacctctatcttggctgtgatagctttgattgctgtgggactgacccagaacaaacctttgccagaaaatgttaagtatgggattgtgttggatgcggggtcatctcacaccaacctgtacatctacaagtggccggccgagaaggagaatgacacaggggtggtgcagcagttagaggaatgccaagtgaaaggtcctggaatctcaaaatatgctcagaaaacagatgaaatcggtgcgtacctggccgaatgcatggaactgtccaccgaactgataccaacatccaagcatcaccagactcctgtctacctgggagccacagcaggcatgcgcttgcttagaatggaaagcgaacaatcggcagacgaggtcctggctgcagtgtcaacaagccttaagagctacccctttgacttccagggtgccaagatcatcactggacaagaggaaggtgcctatgggtggattactattaactatctgctgggcagattcactcaggaacagagttggctaagcctcatctcagacagtcagaaacaggaaacctttggcgctttggatctcggcggagcctccacacagatcaccttcgtgccccaaaacagcactatagagtccccagaaaactctctgcaattccgtctctatggcgaggactatactgtgtacacacacagcttcctgtgctatgggaaggatcaggctctctggcagaaactggccaaggacattcaggtttcaagtggtggcgtccttaaggacccatgctttaacccaggatacgagaaggttgtgaatgtaagtgagctctatggcactccctgcaccaaaagattcgaaaagaagctaccatttgatcagtttcgaatccagggcactggagactacgaacagtgccaccagagcatccttgagctcttcaacaacagccactgcccttactcccagtgtgccttcaatggcgtcttcctgccacctctccatgggagttttggggcgttttctgctttctactttgtgatggatttttttaagaaggtagcgaaaaacagtgtcatctctcaggagaaaatgaccgagataacaaaaaatttttgctcaaaatcttgggaagagacaaagacatcttatccttcagtaaaggagaagtacctgagtgagtactgcttctcgggcgcctacatcctctctctcctgcaaggctataacttcacagacagctcctgggaacagattcattttatgggcaagatcaaagacagcaacgcggggtggactttgggctacatgctgaacttgaccaacatgatcccagctgaacagccgttgtccccgcctctccctcactccacctacatcggcctcatggttctcttctccctgctcttggttgctgtggccatcacaggcctgttcatctatagcaagccttcatatttctggaaagaggcagtaggaagcggagagggcaggggaagtcttctaacatgcggggacgtggaggaaaatcccggccccatgcgtcccgcggccgctaaggtacccaagtggctgcttctcgcactgagcgctctactaccacagtggcccgcagccagtgcctgggagctcacgatcctgcacacaaacgacgtgcacagccggctagagcagaccagcgatgactccaccaagtgcctcaacgccagcctgtgtgtgggcggcgtggcccggctctttaccaaggtgcagcagatccgcaaggaagaacccaacgtgctgtttttggatgccggagaccagtaccagggcaccatctggttcaccgtttacaaaggccttgaagtggcacacttcatgaacatcctgggctacgatgctatggcactgggaaatcatgaatttgataacggtgtggaaggactgattgatcccctcctcagaaacgttaaatttccaattctgagcgcaaacattaaggcacgggggcctctagcacatcagatatctggactttttctgccatctaaagttctctctgttggcggtgaggttgtggggattgttggatatacttcaaaggaaacccctttcctctcaaatccagggacaaatttagtctttgaagatgaaatctctgcattgcagcctgaagtagataaactaaagacgctaaatgtgaataagatcatcgccctggggcactctggttttgagatggacaaacttatcgctcagaaagttcgaggtgtggacatcgtggtgggaggacactccaacacctttctctacacaggaaatccaccttccaaagaagtgcctgcggggaagtacccattcatagtcaccgcagatgatggacggcaggtgcctgtggtccaggcctatgcctttggcaaatacctgggctacctgaaggttgagtttgatgataaaggcaatgttatcacttcctatggaaatcccattcttctcaacagcagcattcctgaagatgcgaccatcaaagcagacattaaccaatggaggataaaattagataattattctacccaggaactcgggagaacgatcgtctacctggatggctccactcagacgtgccgcttcagggaatgcaacatgggaaacctgatctgtgatgccatgattaacaacaacctcagacacccagatgaaatgttttggaaccacgtgtccatgtgcattgtaaacggaggtggcatccggtcccccattgatgagaagaacaatggtaccatcacctgggagaacctggctgctgtgctgccctttggagggacatttgacctcgtccaattaaaagggtccaccctgaagaaggcttttgagcacagcgtgcatcgctatggccagtccaccggagagttcctgcaagtgggtggaatccatgtggtgtacgatattaaccgaaagccctggaacagagtggtccaattagaagttctctgcaccaagtgtcgagtgcccatctatgagcctcttgaaatggataaagtgtataaagtgaccctcccaagctatctggccaacggtggagatggattccagatgataaaagatgaattactaaagcatgactctggtgatcaagatatcagcgtggtttctgaatacatctcaaaaatgaaagtagtttacccagccgttgaagggcggatcaagttctctgcagcaagtcattaccagggaagctttcctttagtaattctttcattttgggcaatgatccttattttgtaccaataa

SEQ ID NO: 39 - DNA, 1746 bp, synthetic construct
  source 1..1746: mol_type = other DNA
  misc_feature 1..1746: note = synthetic construct encoding mouse IL-10 under control of EF1alpha promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 40 - protein, 355 aa, Homo sapiens
  REGION 1..355: note = Galectin-9 protein
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 41 - DNA, 1068 bp, Homo sapiens
  source 1..1068: mol_type = unassigned DNA
  misc_feature 1..1068: note = Galectin-9 DNA
atggccttcagcggttcccaggctccctacctgagtccagctgtccccttttctgggactattcaaggaggtctccaggacggacttcagatcactgtcaatgggaccgttctcagctccagtggaaccaggtttgctgtgaactttcagactggcttcagtggaaatgacattgccttccacttcaaccctcggtttgaagatggagggtacgtggtgtgcaacacgaggcagaacggaagctgggggcccgaggagaggaagacacacatgcctttccagaaggggatgccctttgacctctgcttcctggtgcagagctcagatttcaaggtgatggtgaacgggatcctcttcgtgcagtacttccaccgcgtgcccttccaccgtgtggacaccatctccgtcaatggctctgtgcagctgtcctacatcagcttccagaacccccgcacagtccctgttcagcctgccttctccacggtgccgttctcccagcctgtctgtttcccacccaggcccagggggcgcagacaaaaacctcccggcgtgtggcctgccaacccggctcccattacccagacagtcatccacacagtgcagagcgcccctggacagatgttctctactcccgccatcccacctatgatgtacccccaccccgcctatccgatgcctttcatcaccaccattctgggagggctgtacccatccaagtccatcctcctgtcaggcactgtcctgcccagtgctcagaggttccacatcaacctgtgctctgggaaccacatcgccttccacctgaacccccgttttgatgagaatgctgtggtccgcaacacccagatcgacaactcctgggggtctgaggagcgaagtctgccccgaaaaatgcccttcgtccgtggccagagcttctcagtgtggatcttgtgtgaagctcactgcctcaaggtggccgtggatggtcagcacctgtttgaatactaccatcgcctgaggaacctgcccaccatcaacagactggaagtggggggcgacatccagctgacccatgtgcagacatag

SEQ ID NO: 42 - protein, 417 aa, Homo sapiens
  REGION 1..417: note = CD155 protein
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGMSRNAIIFLVLGILVFLILLGIGIYFYWSKCSREVLWHCHLCPSSTEHASASANGHVSYSAVSRENSSSQDPQTEGTR

SEQ ID NO: 43 - DNA, 1254 bp, Homo sapiens
  source 1..1254: mol_type = unassigned DNA
  misc_feature 1..1254: note = CD155 DNA
atggcccgagccatggccgccgcgtggccgctgctgctggtggcgctactggtgctgtcctggccacccccaggaaccggggacgtcgtcgtgcaggcgcccacccaggtgcccggcttcttgggcgactccgtgacgctgccctgctacctacaggtgcccaacatggaggtgacgcatgtgtcacagctgacttgggcgcggcatggtgaatctggcagcatggccgtcttccaccaaacgcagggccccagctattcggagtccaaacggctggaattcgtggcagccagactgggcgcggagctgcggaatgcctcgctgaggatgttcgggttgcgcgtagaggatgaaggcaactacacctgcctgttcgtcacgttcccgcagggcagcaggagcgtggatatctggctccgagtgcttgccaagccccagaacacagctgaggttcagaaggtccagctcactggagagccagtgcccatggcccgctgcgtctccacagggggtcgcccgccagcccaaatcacctggcactcagacctgggcgggatgcccaatacgagccaggtgccagggttcctgtctggcacagtcactgtcaccagcctctggatattggtgccctcaagccaggtggacggcaagaatgtgacctgcaaggtggagcacgagagctttgagaagcctcagctgctgactgtgaacctcaccgtgtactaccccccagaggtatccatctctggctatgataacaactggtaccttggccagaatgaggccaccctgacctgcgatgctcgcagcaacccagagcccacaggctataattggagcacgaccatgggtcccctgccaccctttgctgtggcccagggcgcccagctcctgatccgtcctgtggacaaaccaatcaacacaactttaatctgcaacgtcaccaatgccctaggagctcgccaggcagaactgaccgtccaggtcaaagagggacctcccagtgagcactcaggcatgtcccgtaacgccatcatcttcctggttctgggaatcctggtttttctgatcctgctggggatcgggatttatttctattggtccaaatgttcccgtgaggtcctttggcactgtcatctgtgtccctcgagtacagagcatgccagcgcctcagctaatgggcatgtctcctattcagctgtgagcagagagaacagctcttcccaggatccacagacagagggcacaaggtga

SEQ ID NO: 44 - protein, 330 aa, Homo sapiens
  REGION 1..330: note = Arginase 1 protein
MSAKSRTIGIIGAPFSKGQPRGGVEEGPTVLRKAGLLEKLKEQVTQNFLILECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 45 - DNA, 993 bp, Homo sapiens
  source 1..993: mol_type = unassigned DNA
  misc_feature 1..993: note = Arginase 1 DNA
atgagcgccaagtccagaaccatagggattattggagctcctttctcaaagggacagccacgaggaggggtggaagaaggccctacagtattgagaaaggctggtctgcttgagaaacttaaagaacaagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

(19) United States
(12) Patent Application Publication
(10) Pub. No.: US 2025/0170184 A1
(43) Pub. Date: May 29, 2025

(54) ENGINEERED IMMUNOMODULATORY ACCESSORY CELLS IMPROVE ALLOGENEIC ISLET TRANSPLANTATION WITHOUT IMMUNOSUPPRESSION

(71) Applicant: CORNELL UNIVERSITY, Ithaca, NY (US)

(72) Inventors: Minglin MA, Ithaca, NY (US); Xi WANG, Boston, MA (US); Bo WANG, Ithaca, NY (US)

(21) Appl. No.: 18/841,883

(22) PCT Filed: Mar. 3, 2023
(86) PCT No.: PCT/US2023/063714

§ 371 Date: Aug. 27, 2024

Related U.S. Application Data
(60) Provisional application No. 63/316,201, filed on Mar. 3, 2022.

(51) Int. Cl. A61K 35/28; A61K 35/39; A61K 38/26; A61K 38/28; A61K 45/06; A61M 37/00; A61P 37/06; C12N 5/071; C12N 5/0775

(52) U.S. Cl. CPC A61K 35/28; A61K 35/39; A61K 38/26; A61K 38/28; A61K 45/06; A61M 37/00; A61P 37/06; C12N 5/0662; C12N 5/0676; A61M 2202/0445; C12N 2510/00

(57) ABSTRACT

Disclosed herein are recombinant mesenchymal stromal cells (e MSCs) that expresses one or more immunomodulatory proteins or polypeptides as well as mixed cell populations that include the eMSCs and one or more cell types distinct of the eMSC, such as islet cells or islets. Also disclosed are implantable cell culture devices that include eMSCs or the mixed cell populations. The eMSCs can be used to improve survival of transplanted cells (such as an allograft), and more particularly to methods of modifying T cell response to an allograft and treating a diabetic subject.

This application claims the priority benefit of U.S. Provisional Patent Application Ser. No. 63/316,201, filed Mar. 3, 2022, which is hereby incorporated by reference in its entirety.

This invention was made with government support under grant 1R01DK105967-01A1 awarded by the National Institutes of Health. The government has certain rights in the invention.

### SEQUENCE LISTING

The Sequence Listing is being submitted electronically in XML format and is hereby incorporated by reference in its entirety. Said XML copy, created on Mar. 2, 2023, is named 147402009121.xml and is 73 KB in Size. No new matter is being introduced.

### FIELD OF USE

This invention relates to recombinant mesenchymal stromal cell that expresses one or more immunomodulatory proteins or peptides, as well as mixed cell populations containing the same, and method of using such recombinant mesenchymal stromal cells and mixed cell populations.

### BACKGROUND

Type 1 diabetes (T1D) is an autoimmune disease in which immune cells (mainly CD8⁺ T cells) mistakenly attack β cells, causing deficiency of insulin and elevation of blood glucose. Replacement of β cells by allogeneic islet transplantation via portal vein has been established in clinics all over the world and shown to improve glycemic control among patients (Shapiro et al., “International Trial of the Edmonton Protocol for Islet Transplantation,” N. Engl. J. Med. 355:1318-1330 (2006); Shapiro et al., “Islet Transplantation in Seven Patients with Type 1 Diabetes Mellitus Using a Glucocorticoid-free Immunosuppressive Regimen,” N. Engl. J. Med. 343:230-238 (2000)). However, systemic immunosuppression, required to prevent allograft rejection, may be toxic to islets and, more importantly, has deleterious side effects to patients (Hernández-Fisac et al., “Tacrolimus-induced Diabetes in Rats Courses with Suppressed Insulin Gene Expression in Pancreatic Islets,” Am. J. Transplant. 7:2455-2462 (2007); Arnold et al., “Association Between Calcineurin Inhibitor Treatment and Peripheral Nerve Dysfunction in Renal Transplant Recipients,” Am. J. Transplant. 13:2426-2432 (2013)). Of note, for most T1D patients, the systemic immunosuppression is riskier than long-term standard management with exogenous insulin supplementation, which makes eliminating systemic immunosuppression critical to β cell replacement therapies. Novel strategies to circumvent the challenges associated with systemic immunosuppression have been extensively pursued for islet transplantation recently including immunoprotection using cell encapsulation devices (Wang et al., “A Nanofibrous Encapsulation Device for Safe Delivery of Insulin-Producing Cells to Treat Type 1 Diabetes,” Sci. Transl. Med. 13, eabb4601 (2021); Liu et al., “A Zwitterionic Polyurethane Nanoporous Device with Low Foreign-body Response for Islet Encapsulation,” Adv Mater 33:e2102852 (2021); Fuchs et al., “Hydrogels in Emerging Technologies for Type 1 Diabetes,” Chem. Rev. 121:11458-11526 (2021); Ernst et al., “Nanotechnology in Cell Replacement Therapies for Type 1 Diabetes,” Adv. Drug Deliver Rev. 139:116-138 (2019)) and induction of local immunotolerance toward allogeneic islets (Wang et al., “Local Immunomodulatory Strategies to Prevent Allo-rejection in Transplantation of Insulin-producing Cells,” Adv. Sci. 8:2003708 (2021)). Compared to cell encapsulation, the local immunomodulation approach is considered as “open,” involving no physical barrier between the graft and the body and therefore can potentially allow better and direct host integration.

In general, T cells play a critical role in allograft rejection (Lakkis et al., “Origin and Biology of the Allogeneic Response,” Cold Spring Harb. Perspect Med. 3:a014993 (2013); Zakrzewski et al., “Overcoming Immunological Barriers in Regenerative Medicine,” Nat. Biotechnol. 32:786-794 (2014)). Upon recognition of alloantigens, a costimulatory signal, commonly provided by B7-1 (CD80) or B7-2 (CD86) ligands on antigen-presenting cells (APCs) that interact with CD28 on T cells, is necessary for T cell activation (Wang et al., “Local Immunomodulatory Strategies to Prevent Allo-rejection in Transplantation of Insulin-producing Cells,” Adv. Sci. 8:2003708 (2021)). Thus, modulation of T cell costimulatory pathways, including blocking T cell costimulation and/or providing negative modulatory signals, has been investigated and used to improve graft survival and functionality. Specifically, the programmed death-1 (PD-1)/programmed death ligand-1 (PD-L1) interaction is a well-studied negative costimulatory pathway, which is critical in maintaining peripheral tolerance and immunological homeostasis (Okazaki et al., “A Rheostat for Immune Responses: The Unique Properties of PD-1 and Their Advantages for Clinical Application,” Nat. Immunol. 14:1212-1218 (2013)). Targeting the PD-1/PD-L1 pathway was shown to regulate and delay immune destruction of allograft in cardiac (Yang et al., “The Novel Costimulatory Programmed Death Ligand 1/B7.1 Pathway Is Functional in Inhibiting Alloimmune Responses in vivo,” J. Immunol. 187:1113-1119 (2011); Dudler et al., “Gene Transfer of Programmed Death Ligand-LIg Prolongs Cardiac Allograft Survival,” Transplantation 82:1733-1737 (2006)), islet (Gao et al., “Stimulating PD-1-negative Signals Concurrent with Blocking CD154 Co-stimulation Induces Long-term Islet Allograft Survival,” Transplantation 76:994-999 (2003)), and corneal (Watson et al., Differential Effects of Costimulatory Pathway Modulation on Corneal Allograft Survival,” Invest. Ophthalmol. Vis. Sci. 47:3417-3422 (2006)) transplantation. Similarly, the cytotoxic T lymphocyte antigen 4 immunoglobulin (CTLA4-Ig) fusion protein, which competitively blocks the CD28-B7 pathways, was shown to inhibit T cell activation (Dumont, “Technology Evaluation: Abatacept, Bristol-Myers Squibb,” Curr. Opin. Mol. Ther. 6:318-330 (2004)) and prevent allograft rejection in skin (Larsen et al., “Long-term Acceptance of Skin and Cardiac Allografts after Blocking CD40 and CD28 Pathways,” Nature 381:434-438 (1996)), cardiac (Turka et al., “T-cell Activation by the CD28 Ligand b7 Is Required for Cardiac Allograft Rejection in vivo,” Proc. Natl. Acad. Sci. U.S.A. 89:11102-11105 (1992); Lin et al., “Long-term Acceptance of Major Histocompatibility Complex Mismatched Cardiac Allografts Induced by CTLA4Ig Plus Donor-specific Transfusion,” J. Exp. Med. 178:1801-1806 (1993)), liver (Li et al., “Costimulation Blockade Promotes the Apoptotic Death of Graft-Infiltrating T Cells and Prolongs Survival of Hepatic Allografts from Flt31-treated Donors,” Transplantation 72:1423-1432 (2001)), and islet (Tran et al., “Distinct Mechanisms for the Induction and Maintenance of Allograft Tolerance with CTLA4-Fc Treatment,” J. Immunol. 159:2232-2239 (1997); Grohmann et al., “CTLA-4-Ig Regulates Tryptophan Catabolism in vivo,” Nat. Immunol. 3:1097-1101 (2002)) transplantation. In addition, PD-L1 and CTLA4-Ig have been demonstrated to inhibit T cell activity in a nonredundant way (Curran et al., “PD-1 and CTLA-4 Combination Blockade Expands Infiltrating T Cells and Reduces Regulatory T and Myeloid Cells within B16 Melanoma Tumors,” Proc. Natl. Acad. Sci. U.S.A. 107:4275-4280 (2010); Fife et al., “Control of Peripheral T-cell Tolerance and Autoimmunity Via the CTLA-4 and PD-1 Pathways,” Immunol. Rev. 224:166-182 (2008)). Despite these promising developments, the PD-L1 or CTLA4-Ig was often administered systemically and caused nonspecific immune responses and immune-related toxicity (Özkaynak et al., “Programmed Death-1 Targeting Can Promote Allograft Survival,” J. Immunol. 169:6546-6553 (2002)). Thus, there is great interest in targeted delivery of immunomodulatory molecules and localized regulation of immune responses within the graft microenvironment.

Multiple studies have reported strategies of using the PD-L1 or CTLA4 immune checkpoint pathways to improve islet transplantation in a localized manner. For example, researchers engineered functional biomaterial platforms [poly(ethylene glycol) (PEG) microgels] to display PD-L1, which have been shown to achieve long-term allogeneic islet graft function in diabetic mouse models with a short-term (15 days) administration of rapamycin (Coronel et al., “Immunotherapy via PD-L1-presenting Biomaterials Leads to Long-term Islet Graft Survival,” Sci. Adv. 6:eaba5573 (2020)). A major advantage of the biomaterial approach is that the biomaterial can be prefabricated, and there is a minimal need, if any, to manipulate or modify the islets. However, biomaterials can cause foreign body responses and induce antibodies (e.g., anti-PEG antibodies) and may be challenging to be applied in current clinical islet transplantation through the portal vein. In addition, the immunomodulatory ligands delivered or presented via biomaterials may degrade or be depleted over time. Alternatively, mouse islets were modified with PD-L1/CTLA4-Ig (Khatib et al., “β-Cell-targeted Blockage of PD1 and CTLA4 Pathways Prevents Development of Autoimmune Diabetes and Acute Allogeneic Islets Rejection,” Gene Ther. 22:430-438 (2015)) or PD-L1 (Batra et al., “Localized Immunomodulation with PD-L1 Results in Sustained Survival and Function of Allogeneic Islets Without Chronic Immunosuppression,” J. Immunol. 204:2840-2851 (2020); Li et al., “PD-L1-driven tolerance protects Neurogenin 3-induced Islet Neogenesis to Reverse Established Type 1 Diabetes in NOD Mice,” Diabetes 64:529-540 (2014)), which resulted in protection of islets from acute rejection. Although modifying islets is a straightforward approach, the modification takes time and may be challenging to be applied in clinical settings, especially given that human islets are not easy to maintain in vitro for a long time.

It would be desirable to develop an approach that overcomes these challenges and protects the graft locally, where such an approach does not require modification of islets and is compatible with current clinical islet transplantation.

The present invention is directed to overcoming these and other deficiencies in the art.

### SUMMARY

A first aspect of the invention relates to a recombinant mesenchymal stromal cell that expresses one or more immunomodulatory proteins or polypeptides.

In one exemplary embodiment, the recombinant mesenchymal stromal cell overexpresses PD-L1 (when compared to a non-recombinant mesenchymal stromal cell) and also expresses the fusion polypeptide CTLA4-Ig.

In another exemplary embodiment, the recombinant mesenchymal stromal cell overexpresses each of CD47, CD39, CD73, and IL-10 (when compared to a non-recombinant mesenchymal stromal cell).

In yet another exemplary embodiment, the recombinant mesenchymal stromal cell overexpresses each of CD39, CD73, IDO1, and IL-10 (when compared to a non-recombinant mesenchymal stromal cell).

A second aspect of the invention relates to a mixed cell population that includes one or more recombinant mesenchymal stromal cells according to the first aspect, and one or more cell types distinct of the recombinant mesenchymal stromal cells. The distinct cell types may be recombinant or non-recombinant in nature, and are preferably one or more islet cells such as a cells, β cells, δ cells, PP cells, and ε cells. The one or more islet cells can be present in the form of an islet or multiple islets, which are present as a mixture with the one or more recombinant mesenchymal stromal cells according to the first aspect.

A third aspect of the invention relates to an implantable cell culture device that includes the mixed cell population according to the second aspect.

A fourth aspect of the invention relates to a method of improving survival of transplanted cells including the step of implanting (i) one or more recombinant mesenchymal stromal cells according to the first aspect, and (ii) one or more cell types distinct of the recombinant mesenchymal stromal cells into an individual at the same locus, whereby the implanted one or more cell types distinct of the recombinant mesenchymal stromal cells exhibit improved survival compared to said one or more cell types implanted in the absence of the one or more recombinant mesenchymal stromal cells at the same locus.

In preferred embodiments, the method is carried out in the absence of administering immunosuppressive agents to the individual or, alternatively, using a reduction in the frequency, quantity, or number of immunosuppressive agents administered to the individual.

A fifth aspect of the invention relates to a method of treating a diabetic subject, which includes the step of implanting the mixed population of cells according to the second aspect into the diabetic subject, whereby the islet cells express insulin and glucagon to treat the diabetic subject.

A sixth aspect of the invention relates to a method of modifying T cell response to an allograft, which includes the step of: implanting an allograft into an individual with one or more recombinant mesenchymal stromal cells according to the first aspect, whereby the one or more recombinant mesenchymal stromal cells cause, relative to an allograft in the absence of the one or more recombinant mesenchymal stromal cells, (i) an increase in the percentage of regulatory T cells (CD4+/CD25+/Foxp3+) present in the implanted graft, and (ii) a reduction in the number of T effector cells (CD4+ or CD8+) present in the implanted graft.

Islet transplantation has been established as a viable treatment modality for type 1 diabetes. However, the side effects of the systemic immunosuppression required for patients often outweigh its benefits. To overcome this problem, mesenchymal stromal cells (eMSCs) were recombinantly engineered to overexpress both PD-L1 and CTLA4-Ig and then used as accessory cells for islet co-transplantation (FIG. 1A). The eMSCs suppressed activation and proliferation of allogeneic and diabetogenic CD4⁺ and CD8⁺ T cells in vitro after 3 days of co-culture. The immunomodulatory function of the PD-L1 and CTLA4-Ig expression was further confirmed by delayed rejection of similarly engineered allogeneic 4T1 cells in immunocompetent mice. The therapeutic potential of the eMSCs was demonstrated in two scenarios of islet transplantation (FIG. 1). In an allogeneic mouse transplantation model, islets transplanted with the eMSCs in kidney capsules functioned and corrected diabetes for up to 100 days without any systemic immunosuppression, while islets transplanted with unmodified MSCs or alone were rejected by days 20 and 14, respectively. Even in a syngeneic model, the eMSCs prolonged and enhanced the islet function, likely due to their anti-inflammatory and paracrine effects. Immunological profiling of explanted allografts with or without eMSCs showed that the eMSCs reduced the infiltration of CD4⁺ or CD8⁺ T effector (T_{eff}) cells and promoted graft infiltration of regulatory T (T_{reg}) cells within the graft microenvironment. It was also confirmed that the immunomodulatory effect was local as allogeneic islets transplanted in the other kidney capsule of the same mouse were still rapidly rejected. These results in mice confirm that PD-L1/CTLA4-Ig-expressing eMSCs can immunologically protect co-transplanted islets. The eMSCs, when used in clinical islet transplantation, should be effective to reduce or minimize the need of systemic immunosuppression and ameliorate its negative impact.

### BRIEF DESCRIPTION OF DRAWINGS

FIGS. 1A-B schematically illustrate local immunotolerance induction by eMSCs. In FIG. 1A, local immunomodulation with eMSCs expressing PD-L1 and CTLA4-Ig protects allogeneic islets from being rejected. FIG. 1B illustrates the experimental design of animal studies showing the syngeneic or allogeneic islets without MSCs, with MSC spheroids, or with eMSC spheroids that were transplanted into diabetic C57BL/6 mice in the kidney capsule.

FIGS. 2A-2N illustrate the in vitro characterization of eMSCs expressing PD-L1 and CTLA4-Ig. FIG. 2A is a schematic illustration of the experimental procedure for generating eMSCs. FIG. 2B is a graph showing mRNA expression of PD-L1 and CTLA4-Ig in MSCs and eMSCs (normalized to GAPDH expression) (n=4). FIG. 2C illustrates a Western blot analysis of PD-L1 and CTLA4-Ig in MSCs and eMSCs. FIG. 2D shows the concentration of CTLA4-Ig in the culture medium of MSCs and eMSCs (n=4). FIG. 2E shows flow cytometric plots of MSCs and eMSCs stained for CD29 and PD-L1. FIG. 2F shows immunofluorescent staining of MSCs and eMSCs with antibodies (green in color version=PD-Li; blue in color version=DAPI).

FIG. 2G shows in vitro CD4⁺ T cell proliferation as measured by CellTrace™ dilution. FIG. 2H shows a quantitative analysis of proliferated CD4⁺ T cell percentage shown in FIG. 2G (n=3). FIG. 2I shows in vitro CD8⁺ T cell proliferation as measured by CellTrace™ dilution. FIG. 2J shows a quantitative analysis of proliferated CD8⁺ T cell percentage shown in FIG. 2I (n=3). FIG. 2K shows a quantitative analysis of activated CD4⁺ T cell percentage (n=3). FIG. 2L shows a quantitative analysis of activated CD8⁺ T cell percentage (n=3). (FIG. 2M contains flow cytometry plots of T cells stained with CD8 and granzyme B. FIG. 2N shows a quantitative analysis of cytotoxic CD8⁺ T cell percentage shown in FIG. 2M. The two-tailed Student's t test was performed when the data consisted of only two groups. One-way ANOVA followed by Tukey's test was performed for comparing the multigroup data. The level of significance was labeled by *, **, ***, and ****, denoting P values of denoting a P value of <0.05, <0.01, <0.001, and <0.0001, respectively.

FIGS. 3A-3G illustrates that expression of PD-L1 and CTLA4-Ig in 4T1 breast cancer cells delays allorejection. FIG. 3A illustrates a pair of flow cytometric dot plots of native 4T1 and modified 4T1 stained for PD-Li. FIG. 3B contains bioluminescent images of healthy C57BL/6 mice transplanted with native 4T1 cells and modified 4T1 cells (derived from fully MHC-mismatched BALB/c) in right hindlimb (n=5). FIG. 3C shows a quantitative analysis of the bioluminescent intensity of the engrafted cells shown in FIG. 3B. Each line represents one mouse (n=5). FIG. 3D is a graft survival curve of healthy C57BL/6 mice receiving native 4T1 and modified 4T1 cells. FIG. 3E includes representative H&E staining images of native 4T1 cells engrafted in syngeneic BALB/c mice (left) and modified 4T1 cells engrafted in allogeneic C57BL/6 mice (right). Representative digital images of 4T1 tumor grown in syngeneic and allogeneic mice in the right hindlimb are shown in the inset. FIG. 3F includes representative immunofluorescent images of native 4T1 cells engrafted in syngeneic BALB/c mice (left) and modified 4T1 cells engrafted in allogeneic C57BL/6 mice (right) stained with antibodies (green in color version=PD-Li; blue in color version=DAPI). FIG. 3G shows flow cytometry analysis of native 4T1 cells isolated from the tumor grown in syngeneic mice and modified 4T1 cells isolated from long-term grafts in allogeneic mice after 60 days with PD-L1 marker. Survival curve was analyzed using a Mantel-Cox test. The level of significance was labeled by *, **, ***, and ****, denoting a P value of <0.05, <0.01, <0.001, and <0.0001, respectively. Scale bars, 100 μm in FIG. 3F.

FIGS. 4A-4I illustrate that PD-L1/CTLA4-Ig-overexpressing eMSCs improve syngeneic islet transplantation in mice. FIG. 4A shows live and dead staining of islets cocultured without MSCs, with MSC spheroids, or with eMSC spheroids in vitro for 24 hours (green in color version=live cells; red in color version=dead cells). FIG. 4B shows a quantitative analysis of fluorescence intensity of images shown in FIG. 4A (n=5). FIG. 4C depicts a stimulation index of islets (the ratio of insulin secretion at high glucose to that at low glucose) cocultured without MSCs, with MSC spheroids, or with eMSC spheroids for 24 hours (n=4 to 5). FIG. 4D shows blood glucose curves of diabetic C57BL/6 mice transplanted with syngeneic islets with a marginal dosage without MSCs (no-MSC group) (n=3), with MSC spheroids (MSC group) (n=4), and with eMSC spheroids (eMSC group) (n=5) in the kidney capsule. FIG. 4E shows graft survival curves of indicated groups shown in FIG. 4D. FIG. 4F shows blood glucose measurement in the intraperitoneal glucose tolerance test of different groups (n=3 to 5). FIG. 4G is an image of representative H&E staining of syngeneic islets with eMSC engrafted in diabetic C57BL/6 mice in the kidney capsule. FIG. 4H is an image of representative immunofluorescent staining of syngeneic islets with eMSCs engrafted in diabetic C57BL/6 mice in the kidney capsule. DAPI (gray in color version), insulin (INS, magenta in color version), and glucagon (GCG, green in color version). FIG. 4I is a higher magnification of islets shown in FIG. 4H. One-way ANOVA followed by Tukey's test was performed for comparing the multigroup data. Survival curve was analyzed using a Mantel-Cox test. The level of significance was labeled by n.s., *, and **, denoting nonsignificant and P values of <0.05 and <0.01, respectively. Scale bars, 50 μm in FIGS. 4A and 4I, and 100 μm in FIGS. 4G and 4H.

FIGS. 5A-5G show that PD-L1/CTLA4-Ig-overexpressing eMSCs delay allogeneic islet rejection in mice. FIG. 5A is a pair of representative digital images of kidneys transplanted with islets alone (top) or with either MSC or eMSC spheroids (bottom). FIG. 5B shows blood glucose curves of diabetic C57BL/6 mice transplanted with BALB/c islets without MSCs (no-MSC group) (n=9), BALB/c islets with MSC spheroids (MSC group) (n=9), and BALB/c islets with eMSC spheroids (eMSC group) (n=9) in the kidney capsule. FIG. 5C shows a graft survival curve of indicated groups shown in FIG. 5B. FIG. 5D shows blood glucose measurement in the intraperitoneal glucose tolerance test of different groups (n=3). FIG. 5E contains bioluminescent images of diabetic C57BL/6 mice transplanted with GFP/luciferase FVB mouse islets without MSCs (no-MSC group) (n=6), with MSC spheroids (MSC group) (n=6), or with eMSC spheroids (eMSC group) (n=8) in the kidney capsule. FIG. 5F shows a quantitative analysis of bioluminescent signals measured in mice with different grafts (n=6 to 8). FIG. 5G is a graft survival curve of indicated groups in FIG. 5F (n=6 to 8). Survival curve was analyzed using a Mantel-Cox test. The level of significance was labeled by *** and ****, denoting P values of <0.001 and <0.0001, respectively.

FIGS. 6A-6N illustrate the characterization of immune cells in the local microenvironment of the islet allografts in mice. FIG. 6A shows the percentage of CD3+ T cells in CD45+ cells (n=4). FIG. 6B shows the percentage of CD4+ Teff cells in CD4+ T cells (n=4). FIG. 6C shows the percentage of CD8+ Teff cells in CD8+ T cells (n=4). FIG. 6D shows the percentage of activated dendritic cells (DCs) in DCs (n=4). FIG. 6E shows the percentage of PD-1+ cells in CD8+ T cells (n=4). FIG. 6F shows the percentage of Treg cells in CD4+ T cells (n=4). FIG. 6G shows the ratio of Treg to CD4+ Teff cells (n=4). FIG. 6H shows the ratio of Treg cells to CD8+ Teff cells (n=4). FIG. 6I is a panel of representative immunofluorescent staining of islet grafts (DAPI=blue in color version; INS=red in color version; CD3=green in color version). FIG. 6J shows the ratio of CD3+ T cells to insulin+3 cells shown in FIG. 6I (n=5). FIG. 6K is a panel of representative immunofluorescent staining of islet grafts (DAPI=blue in color version; CD4=red in color version; CD3=green in color version). FIG. 6L shows a quantitative analysis of CD4+ T cell density shown in FIG. 6K (n=5). FIG. 6M is a panel of representative immunofluorescent staining of islet grafts (DAPI, blue; CD8, red; CD3, green). FIG. 6N shows a quantitative analysis of CD8+ T cell density shown in 6M (n=5). One-way ANOVA followed by Tukey's test was performed for comparing the multigroup data. The level of significance was labeled by n.s., *, **, ***, and ****, denoting nonsignificant and P values of <0.05, <0.01, <0.001, and <0.0001, respectively. Scale bars, 50 μm in FIGS. 6I, 6K, and 6M.

FIGS. 7A-7G illustrate the ex vivo characterization of allografts with eMSCs. FIG. 7A is a representative H&E image of islet grafts with eMSCs explanted on day 45 (higher-magnification image on the right). The asterisk indicates the allogeneic islet. FIG. 7B is a representative immunofluorescent staining of islet grafts with eMSCs explanted on day 45 with markers DAPI (gray in color version), insulin (INS, magenta in color version), and glucagon (GCG, green in color version). A higher-magnification image is provided on the right. FIG. 7C is a representative immunofluorescent staining of islet grafts with eMSCs explanted on day 45 with markers DAPI (gray in color version), insulin (INS, magenta in color version), and Foxp3 (green in color version). FIG. 7D contains a pair of higher-magnification images from FIG. 7C. FIG. 7E contains a pair of representative immunofluorescent staining of islet grafts with eMSCs explanted on day 45 with markers DAPI (gray in color version), CD4 (magenta in color version), and Foxp3 (green in color version). FIG. 7F contains a pair of representative immunofluorescent staining of islet grafts with eMSCs explanted on day 103. Left: DAPI (gray in color version), insulin (INS, magenta in color version), and Foxp3 (green in color version). Right: DAPI (gray in color version), CD4 (magenta in color version), and Foxp3 (green in color version). FIG. 7G shows Foxp3+ Treg cell density within the islet grafts (n=3 for no-MSC and MSC groups, grafts retrieved on day 30; and n=4 for eMSC group, grafts retrieved between 45 and 103 days). FIG. 7H shows the percentage of Foxp3+ Treg cells in the CD4+ T cell population within the retrieved grafts (n=3 for no-MSC and MSC groups, grafts retrieved on day 30; and n=4 for the eMSC group, grafts retrieved between 45 and 103 days). One-way ANOVA followed by Tukey's test was performed for comparing the multigroup data. The level of significance was labeled by ** and ****, denoting P values of <0.01 and <0.0001, respectively. Scale bars, 50 μm FIGS. 7D-7F and 100 μm 7A-7C.

FIG. 8 shows the immunomodulation induced by eMSCs is a local effect when the allogeneic islets and the eMSCs were transplanted separately into two kidneys in diabetic C57BL/6 mice. Blood glucose curves of mice receiving allogeneic islets and the eMSCs in two kidneys (n=5).

FIGS. 9A-9C illustrate host immune responses of diabetic C57BL/6 mice receiving allogeneic islets in the kidney capsule. Representative H&E images of allografts retrieved on day 5 (FIG. 9A), 8 (FIG. 9B) and 15 (FIG. 9C) after transplantation. Stars indicate islets. Scale bar: 100 μm.

FIGS. 10A-10I show the host immune responses of diabetic C57BL/6 mice receiving allogeneic islets in the kidney capsule. Representative immunofluorescent images of allografts retrieved on day 5 (FIG. 10A) and day 8 (FIG. 10B) after transplantation stained with insulin (INS, red in color version), CD3 (green in color version) and DAPI (blue in color version). Magnified field is shown on the right. FIG. 10C is a graph showing the ratio of CD3⁺ T cells to insulin⁺β cells measured from immunofluorescent images of grafts in FIGS. 10A-10B (n=5). Representative immunofluorescent images of allografts retrieved on day 5 (FIG. 10D) and day 8 (FIG. 10E) after transplantation stained with CD4 (red in color version), CD3 (green in color version) and DAPI (blue in color version). Magnified field is shown on the right. FIG. 10F is a graph showing quantitative analysis of the density of CD4⁺ T cells measured from immunofluorescent images of grafts in FIGS. 10D-10E (n=5). Representative immunofluorescent images of allografts retrieved on day 5 (FIG. 10G) and day 8 (FIG. 10H) after transplantation stained with CD8 (red in color version), CD3 (green in color version) and DAPI (blue in color version). Magnified field is shown on the right. FIG. 10I is a graph showing quantitative analysis of the density of CD8⁺ T cells measured from immunofluorescent images of grafts in FIGS. 10G-10H (n=5). The two-tailed Student's t test was performed. The level of significance was labeled by * and **, denoting p value<0.05 and 0.01, respectively. Scale bar: 50 μm (FIGS. 10A, 10B, 10D, 10E, 10G and 10H).

FIGS. 11A-11D are graphs illustrating CD4 and CD8 T cell analysis within the local microenvironment of allogeneic islet graft. In FIG. 11A, the percentage of CD4⁺ T cells in CD45⁺ cell population is shown (n=4). In FIG. 11B, the percentage of CD8⁺ T cells in CD45+ cell population is shown (n=4). In FIG. 11C, the percentage of CD4⁺ T cells in CD3⁺ T cell population is shown (n=4). In FIG. 11D, the percentage of CD8⁺ T cells in CD3⁺ T cell population is shown (n=4). The one-way ANOVA followed by Tukey's test was performed for comparing the multigroup data. The level of significance was labeled by n.s., *, ** denoting non-significant, p value of <0.05, <0.01 and <0.001, respectively.

FIGS. 12A-D show the histological analysis of allograft with eMSCs. FIG. 12A shows representative immunofluorescent staining of islet grafts with eMSCs explanted on day 45 with markers DAPI (gray in color version), insulin (INS, red in color version) and Foxp3 (green in color version). FIG. 12B shows representative immunofluorescent staining of islet grafts with eMSCs explanted on day 45 with markers DAPI (gray in color version), CD4 (red in color version) and Foxp3 (green in color version). FIG. 12C shows representative immunofluorescent staining of islet grafts with eMSCs explanted on day 103. DAPI (gray in color version), insulin (INS, red in color version) and Foxp3 (green in color version). FIG. 12D shows representative immunofluorescent staining of islet grafts with eMSCs explanted on day 103. DAPI (gray in color version), CD4 (red in color version) and Foxp3 (green in color version). Scale bar: 50 μm.

FIGS. 13A-D illustrate the in vitro characterization of eMSCs expressing IL-10, confirming the IL-10 eMSCs inhibit allogenic response in vitro. FIGS. 13A-13B show that the IL-10 expressing eMSCs significantly reduce proliferation of CD4⁺ and CD8⁺ T cells, respectively. FIGS. 13C-13D show that the IL-10 expressing eMSCs significantly reduce activation of CD4⁺ and CD8⁺ T cells, respectively.

FIG. 14 is a recombinant gene construct encoding mouse IDO1 and IL-10 (SEQ ID NO: 37). The gene construct includes an EF1a promoter (gray shading) followed by a Kozak sequence immediately upstream of the mouse IDO1 open reading frame (bold) and mouse IL-10 open reading frame (bold), which are separated by a T2A sequence (dashed underline).

FIG. 15 is a recombinant gene construct encoding mouse CD39 and CD73 (SEQ ID NO: 38). The gene construct includes an EF1a promoter (gray shading) followed by a Kozak sequence immediately upstream of the mouse CD39 open reading frame (bold) and mouse CD73 open reading frame (bold), which are separated by a T2A sequence (dashed underline).

FIG. 16 is a recombinant gene construct encoding mouse IL-10 (SEQ ID NO: 40). The gene construct includes an EF1a promoter (gray shading) followed by a Kozak sequence immediately upstream of the mouse IL-10 open reading frame (bold).

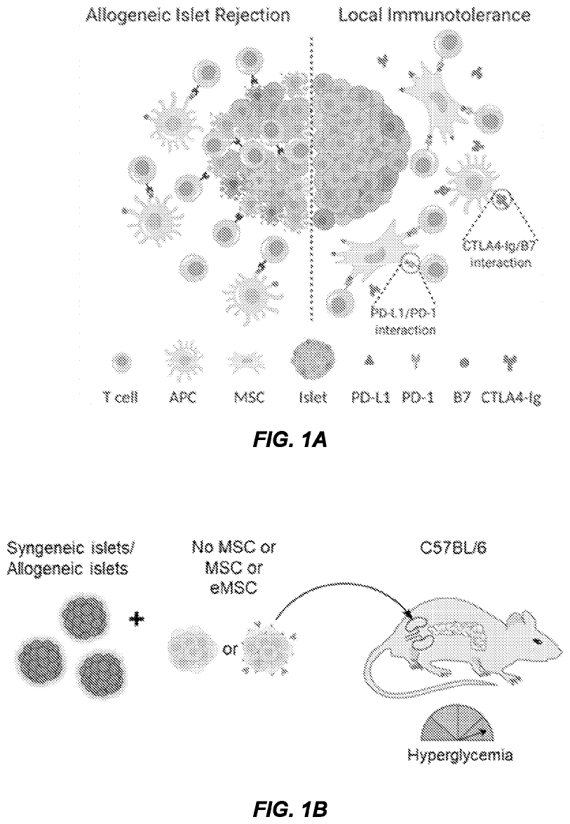

### DETAILED DESCRIPTION

One aspect of the invention relates to a recombinant (or engineered) mesenchymal stromal cell (eMSC) that expresses one or more immunomodulatory proteins or polypeptides. The eMSCs can be used to form mixed cell populations, and to improve survival of transplanted cells (included in mixed cell populations) by modifying T cell response to transplanted cells such as an allograft.

Mesenchymal stem cells (MSCs) have the ability to modulate the immune system and are multipotent. MSCs can readily be isolated from different sources, including the umbilical cord, cord blood, adipose tissue, and bone marrow. See “Mesenchymal Stem Cells: Methods and Protocols,” In: Methods in Molecular Biology 449, Prockop et al. (Eds.), Humana Press (2008); Francis et al., “Isolating Adipose-derived Mesenchymal Stem Cells from Lipoaspirate Blood and Saline Fraction,” Organogenesis 6:11-14 (2010); Secco et al., “Multipotent Stem Cells from Umbilical Cord: Cord Is Richer Than Blood!” Stem Cells 26:146-150 (2008); Ghorbani et al., “Isolation of Adipose Tissue Mesenchymal Stem Cells Without Tissue Destruction: A Non-enzymatic Method,” Tissue Cell 46(1):54-8 (2014); Baer et al., “Adipose-Derived Mesenchymal Stromal/Stem Cells: Tissue Localization, Characterization, and Heterogeneity,” Stem Cells Internat'l 2012: Article ID 812693, 11 pp (2012), each of which is hereby incorporated by reference in its entirety.

Subsequent to isolation of MSCs, the MSCs can be recombinantly modified to express one or more immunomodulatory proteins or polypeptides including combinations of the immunomodulatory proteins or polypeptides. As used herein, the terms ‘recombinant MSCs’ and ‘engineered MSCs’ (or eMSCs) are used interchangeably.

In certain embodiments, immunomodulatory proteins or polypeptides comprise proteins or polypeptides capable of modifying or regulating one or more immune functions, including, but not limited to, production and action of cells that fight disease or infection.

In certain embodiments, the one or more immunomodulatory proteins or polypeptides comprise a combination of at least one immunomodulatory protein or polypeptide expressed on the surface of the eMSCs and at least one immunomodulatory protein or polypeptide that is secreted by the eMSCs.

Exemplary immunomodulatory proteins include, without limitation, the following immunosuppressive proteins or polypeptides: programmed death ligand-1 (PD-L1), cytotoxic T lymphocyte antigen 4 immunoglobulin (CTLA4-Ig) fusion protein, CD47, CD39, CD73, IL-10, IDO1, Galectin-9, CD155, and Arg1.

In the case of immunomodulatory proteins or polypeptides that are normally turned off or expressed at low levels in the MSCs, then the eMSCs will overexpress such one or more immunomodulatory proteins or polypeptides. In the case of immunomodulatory proteins or polypeptides that take the form of non-naturally occurring fusion proteins, then such immunomodulatory fusion proteins are necessarily overexpressed in comparison to the MSCs.

In certain embodiments, the one or more immunomodulatory proteins or polypeptides are more than 100-fold overexpressed by the eMSCs (in comparison to the MSCs), including more than 200-fold overexpressed, more than 300-fold overexpressed, more than 400-fold overexpressed, more than 500-fold overexpressed, more than 600-fold overexpressed, more than 700-fold overexpressed, more than 800-fold overexpressed, more than 900-fold overexpressed, or more than 1000-fold overexpressed.

As discussed below, recombinant modification of the MSCs can be carried out using several approaches. In one approach, an exogenous transgene can be introduced into the MSCs, thus forming eMSCs, where the transgene includes a promoter sequence, an opening reading frame encoding one or more of the immunomodulatory proteins or polypeptides, and 3′ untranslated regions. The promoter and 3′ untranslated regions can be selected to achieve appropriate expression of the one or more of the immunomodulatory proteins or polypeptides. In another approach, an exogenous promoter can be introduced into the promoter region of the genomic DNA encoding one or more of the immunomodulatory proteins that are unexpressed or minimally expressed in the MSCs, thereby altering expression of the recombinant gene to cause overexpression thereof.

PD-L1 (CD274) plays a critical role in induction and maintenance of immune tolerance to self and has been shown to modulate the activation threshold of T-cells and limit T-cell effector response (Freeman et al., “Engagement of the PD-1 Immunoinhibitory Receptor by a Novel B7 Family Member Leads to Negative Regulation of Lymphocyte Activation,” J. Exp. Med. 192(7):1027-1034 (2000), which is hereby incorporated by reference in its entirety).

One exemplary human PD-L1 protein comprises the amino acid sequence according to SEQ ID NO: 1 shown below:

MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET

This human PD-L1 protein is encoded by the nucleic acid molecule according to SEQ ID NO: 2 below:

| | |
|---|---|
| 1 | atgaggatat ttgctgtctt tatattcatg acctactggc atttgctgaa cgcatttact |
| 61 | gtcacggttc ccaaggacct atatgtggta gagtatggta gcaatatgac aattgaatgc |
| 121 | aaattcccag tagaaaaaca attagacctg gctgcactaa ttgtctattg ggaaatggag |
| 181 | gataagaaca ttattcaatt tgtgcatgga gaggaagacc tgaaggttca gcatagtagc |
| 241 | tacagacaga gggcccggct gttgaaggac cagctctccc tgggaaatgc tgcacttcag |
| 301 | atcacagatg tgaaattgca ggatgcaggg gtgtaccgct gcatgatcag ctatggtggt |
| 361 | gccgactaca agcgaattac tgtgaaagtc aatgccccat acaacaaaat caaccaaaga |
| 421 | attttggttg tggatccagt cacctctgaa catgaactga catgtcaggc tgagggctac |
| 481 | cccaaggccg aagtcatctg gacaagcagt gaccatcaag tcctgagtgg taagaccacc |
| 541 | accaccaatt ccaagagaga ggagaagctt ttcaatgtga ccagcacact gagaatcaac |
| 601 | acaacaacta atgagatttt ctactgcact tttaggagat tagatcctga ggaaaaccat |
| 661 | acagctgaat tggtcatccc agaactacct ctggcacatc ctccaaatga aaggactcac |
| 721 | ttggtaattc tgggagccat cttattatgc cttggtgtag cactgacatt catcttccgt |
| 781 | ttaagaaaag ggagaatgat ggatgtgaaa aaatgtggca tccaagatac aaactcaaag |
| 841 | aagcaaagtg atacacattt ggaggagacg taa |

Both the protein and encoding nucleic acid molecule are disclosed in Genbank Accession AY254342, which is hereby incorporated by reference in its entirety.

A number of PD-L1 homologs exist in other species and can be used to practice the present invention. These include, without limitation, chimpanzee (>99% sequence identity to human, Genbank Accession XP_001140705); orangutan (>98% sequence identity to human, Genbank Accession XP_002819859.1); macaque (>93% sequence identity to human, Genbank Accession XP_005581836); rhesus monkey (>92% sequence identity to human, Genbank Accession AB033163); gorilla (>92% sequence identity to human, Genbank Accession XP_018889139); horse (>79% sequence identity to human, Genbank Accession XP_001492892); dog (>75% sequence identity to human, Genbank Accession NM_001291972); cow (>73% sequence identity to human, Genbank Accession LC271174); cat (73% sequence identity to human, Genbank Accession LC735019); and mouse (>69% sequence identity to human, Genbank Accession GQ904196). Each of the above-identified Genbank Accessions is hereby incorporated by reference in its entirety.

Based on the foregoing, it is contemplated that variations of the human PD-L1 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 1 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human PD-L1 protein can also be tolerated if activity of the protein is not severely compromised.

CTLA4-Ig is an Fc fusion protein containing the extracellular domain of CTLA-4 (CD152), a receptor known to deliver a negative signal to T cells. CTLA4-Ig modulates T cell costimulatory signals by blocking the CD80 and CD86 ligands from binding to CD28, which delivers a positive T cell costimulatory signal. Xu et al., “Affinity and Cross-reactivity Engineering of CTLA4-Ig to Modulate T Cell Costimulation”, J Immunol 189(9):4470-7 (2012), which is hereby incorporated by reference in its entirety, reports a number of CTLA4-Ig single amino acid substitution variants. Variants were identified that equally affected the binding affinity of CTLA4-Ig for both ligands as well as those that differentially affected binding. All of the high-affinity variants showed improved off-rates, with the best one being a 17.5-fold improved off-rate over parental CTLA4-Ig binding to CD86. Allostimulation of human CD4(+) T cells showed that improvement of CD80 and CD86 binding activity augmented inhibition of naive and primed T cell activation. In general, increased affinity for CD86 resulted in more potent inhibition of T cell response than did increased affinity for CD80. In addition, Belatacept—a second-generation CTLA4-Ig protein with higher in-vitro potency—was approved by the FDA in 2011 for maintenance immunosuppression in kidney transplant recipients.

The CTLA4-Ig Fc fusion protein contains the CTLA-4 extracellular domain and an IgG constant region (Fc). A number of exemplary CTLA4-Ig Fc fusion protein are described in U.S. Pat. No. 10,155,800, which is hereby incorporated by reference in its entirety.

One exemplary human CTLA4 protein comprises the amino acid sequence according to SEQ ID NO: 3 shown below:

MACLGFQRHKAQLNLATRTWPCTLLFFLLFIPVFCKAMHVAQPAVVLAS
SRGIASFVCEYASPGKATEVRVTVLRQADSQVTEVCAATYMMGNELTFL
DDSICTGTSSGNQVNLTIQGLRAMDTGLYICKVELMYPPPYYLGIGNGT
QIYVIDPEPCPDSDFLLWILAAVSSGLFFYSFLLTAVSLSKMLKKRSPL
TTGVYVKMPPTEPECEKQFQPYFIPIN

This human CTLA4 protein a signal sequence at amino acids 1-39, an extracellular domain at amino acids 40-161 (bold), a transmembrane domain at amino acids 162-182, and a topological domain at amino acids 183-223. The full length CTLA4 protein is encoded by the nucleic acid molecule according to SEQ ID NO: 4 below:

| | |
|---|---|
| 1 | atggcttgcc ttggatttca gcggcacaag gctcagctga acctggctac caggacctgg |
| 61 | ccctgcactc tcctgttttt tcttctcttc atccctgtct tctgcaaagc aatgcacgtg |
| 121 | gcccagcctg ctgtggtact ggccagcagc cgaggcatcg ccagctttgt gtgtgagtat |
| 181 | gcatctccag gcaaagccac tgaggtccgg gtgacagtgc ttcggcaggc tgacagccag |
| 241 | gtgactgaag tctgtgcggc aacctacatg atggggaatg agttgacctt cctagatgat |
| 301 | tccatctgca cgggcacctc cagtggaaat caagtgaacc tcactatcca aggactgagg |
| 361 | gccatggaca cgggactcta catctgcaag gtggagctca tgtacccacc gccatactac |
| 421 | ctgggcatag gcaacggaac ccagatttat gtaattgatc cagaaccgtg cccagattct |
| 481 | gacttcctcc tctggatcct tgcagcagtt agttcggggt tgttttttta tagctttctc |
| 541 | ctcacagctg tttctttgag caaaatgcta aagaaaagaa gccctcttac aacaggggtc |
| 601 | tatgtgaaaa tgcccccaac agagccagaa tgtgaaaagc aatttcagcc ttattttatt |
| 661 | cccatcaatt ga |

The protein and encoding nucleic acid molecule are disclosed in Genbank Accessions P16410 and NM_005214, each of which is hereby incorporated by reference in its entirety.

A number of CTLA4 homologs exist in other species and can be used to practice the present invention. These include, without limitation, chimpanzee (100% sequence identity to human, Genbank Accession PNI67063); orangutan (>99% sequence identity to human, Genbank Accession XP_002812816); macaque (>96% sequence identity to human, Genbank Accession NP_001038204.1); rhesus monkey (>96% sequence identity to human, Genbank Accession NP_001038204); gorilla (100% sequence identity to human, Genbank Accession XP_004033133); horse (>86% sequence identity to human, Genbank Accession XP_023478240); dog (>87% sequence identity to human, Genbank Accession AAF23813); cow (>84% sequence identity to human, Genbank Accession BAX73996.1); cat (>87% sequence identity to human, Genbank Accession NP_001009236); and mouse (>74% sequence identity to human, Genbank Accession AAF01489.1). Each of the above-identified Genbank Accessions is hereby incorporated by reference in its entirety.

Based on the foregoing, it is contemplated that variations of the human CTLA4 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 3 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human CTLA4 protein can also be tolerated if activity of the protein is not severely compromised.

The IgG constant region (Fc) used to prepare the fusion protein can be any of a variety of human IgG constant regions, including constant regions from IgG1, IgG2, IgG3, or IgG4. The IgG constant region preferably contains an Fc region containing Cys to Ser mutations as described in Xu et al., “Affinity and Cross-reactivity Engineering of CTLA4-Ig to Modulate T Cell Costimulation,” J Immunol 189(9):4470-7 (2012), which is hereby incorporated by reference in its entirety. Exemplary IgG constant regions that have been previously used to create CTLA4-Ig Fc fusion proteins are disclosed in the Xu et al. publication as well as U.S. Pat. No. 10,300,112, which is hereby incorporated by reference in its entirety, Any of those can be utilized in practicing the present invention.

CD47 is a cell-surface antigen that acts as an antiphagocytic signal, via the CD47-SIPRα signaling pathway, that cancer cells employ to inhibit macrophage-mediated destruction. Upon CD47 engagement with SIRPα, the intracellular immunoreceptor tyrosine-based inhibition motif (ITIM) domain of SIRPα becomes phosphorylated, leading to recruitment and activation of src homology regions 2 domain-containing phosphatases, which inhibits phagocytosis of CD47-expressing cells. Early clinical trials of anti-CD47 antibody Hu5F9-G4 showed promising results in patients with non-Hodgkin's lymphoma, highlighting the contribution of CD47-SIRPα signaling pathway to tumor immune evasion. Additionally, multiple recent studies also demonstrate that CD47 blockade synergize with T cell-mediated anti-tumor immunity, indicating the potential synergy between CD47- and PD-1/CTLA-4-mediated immunosuppression to alleviate allorejection.

One exemplary human CD47 protein comprises the amino acid sequence according to SEQ ID NO: 7 shown below:

MWPLVAALLLGSACCGSAQLLFNKTKSVEFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWFSPNENILIVI
FPIFAILLFWGQFGIKTLKYRSGGMDEKTIALLVAGLVITVIVIVGAIL
FVPGEYSLKNATGLGLIVISTGILILLHYYVFSTAIGLTSFVIAILVIQ
VIAYILAVVGLSLCIAACIPMHGPLLISGLSILALAQLLGLVYMKFVAS
NQKTIQPPRKAVEEPLNAFKESKGMMNDE

This human CD47 protein includes a signal sequence at amino acids 1-18, and the mature protein spans amino acids 19-323. The full length CD47 protein is encoded by the nucleic acid molecule according to SEQ ID NO: 8 below:

| | |
|---|---|
| 1 | atgtggcccc tggtagcggc gctgttgctg ggctcggcgt gctgcggatc agctcagcta |
| 61 | ctatttaata aaacaaaatc tgtagaattc acgttttgta atgacactgt cgtcattcca |
| 121 | tgctttgtta ctaatatgga ggcacaaaac actactgaag tatacgtaaa gtggaaattt |
| 181 | aaaggaagag atatttacac ctttgatgga gctctaaaca agtccactgt ccccactgac |
| 241 | tttagtagtg caaaaattga agtctcacaa ttactaaaag gagatgcctc tttgaagatg |
| 301 | gataagagtg atgctgtctc acacacagga aactacactt gtgaagtaac agaattaacc |
| 361 | agagaaggtg aaacgatcat cgagctaaaa tatcgtgttg tttcatggtt ttctccaaat |
| 421 | gaaaatattc ttattgttat tttcccaatt tttgctatac tcctgttctg gggacagttt |
| 481 | ggtattaaaa cacttaaata tagatccggt ggtatggatg agaaaacaat tgctttactt |
| 541 | gttgctggac tagtgatcac tgtcattgtc attgttggag ccattctttt cgtcccaggt |
| 601 | gaatattcat taaagaatgc tactggcctt ggtttaattg tgacttctac agggatatta |
| 661 | atattacttc actactatgt gtttagtaca gcgattggat taacctcctt cgtcattgcc |
| 721 | atattggtta ttcaggtgat agcctatatc ctcgctgtgg ttggactgag tctctgtatt |
| 781 | gcggcgtgta taccaatgca tggccctctt ctgatttcag gtttgagtat cttagctcta |
| 841 | gcacaattac ttggactagt ttatatgaaa tttgtggctt ccaatcagaa gactatacaa |
| 901 | cctcctagga aagctgtaga ggaacccctt aatgcattca aagaatcaaa aggaatgatg |
| 961 | aatgatgaat aa |

The protein and encoding nucleic acid molecule are disclosed in Genbank Accessions NP_001768 and NM_001777, each of which is hereby incorporated by reference in its entirety.

A number of CD47 homologs exist in other species and can be used to practice the present invention. These include, without limitation, chimpanzee (>99% sequence identity to human, Genbank Accession XP_016797086); orangutan (>99% sequence identity to human, Genbank Accession NP_001124828); macaque (>98% sequence identity to human, Genbank Accession XP_005548289); rhesus monkey (>98% sequence identity to human, Genbank Accession NP_001253446); gorilla (>99% sequence identity to human, Genbank Accession XP_030865189); horse (>69% sequence identity to human, Genbank Accession XP_023479584); dog (>71% sequence identity to human, Genbank Accession XP_038300507); cow (>72% sequence identity to human, Genbank Accession XP_005201320); cat (>69% sequence identity to human, Genbank Accession XP_044892827); and mouse (>64% sequence identity to human, Genbank Accession XP_006521872). Each of the above-identified Genbank Accessions is hereby incorporated by reference in its entirety.

Based on the foregoing, it is contemplated that variations of the human CD47 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 7 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human CD47 protein can also be tolerated if activity of the protein is not severely compromised.

CD39 is a cell-surface bound ectoenzyme that acts as an ATP diphosphohydrolase and CD73 is a cell-surface bound ectoenzyme that acts as a 5-prime-nucleotidase that catalyzes the conversion at neutral pH of purine 5-prime mononucleotides to nucleosides (typically AMP to adenosine). Both CD39 and CD73 are involved in the adenosine pathway. Adenosine pathway is another clinically proven immunosuppressive pathway that tumors harness for immune evasion. Oxygen deprivation in the tumor microenvironment limits the availability of energy sources and induce the accumulation of extracellular ATP. Upregulated expression of CD39 and CD73 in numerous categories of tumor cells, and also often on various immune cells in the tumor microenvironment, degrades ATP to adenosine, which binds to one of four adenosine receptors, A1R, A2AR, A2BR, and A3R, mediating profound immunosuppression via multiple mechanisms. Adenosine receptor antagonists have shown clinical efficacy for cancer treatment, highlighting the importance of adenosine-mediated immunosuppression for tumor development. Therefore, adenosine pathway is a promising target for mitigation of allorejection.

One exemplary human CD39 protein comprises the amino acid sequence according to SEQ ID NO: 9 shown below:

MEDTKESNVKTFCSKNILAILGESSIIAVIALLAVGLTQNKALPENVKY
GIVLDAGSSHTSLYIYKWPAEKENDTGVVHQVEECRVKGPGISKFVQKV
NEIGIYLTDCMERAREVIPRSQHQETPVYLGATAGMRLLRMESEELADR
VLDVVERSLSNYPFDFQGARIITGQEEGAYGWITINYLLGKFSQKTRWF
SIVPYETNNQETFGALDLGGASTQVTFVPQNQTIESPDNALQFRLYGKD
YNVYTHSFLCYGKDQALWQKLAKDIQVASNEILRDPCFHPGYKKVVNVS
DLYKTPCTKRFEMTLPFQQFEIQGIGNYQQCHQSILELFNTSYCPYSQC
AFNGIFLPPLQGDFGAFSAFYFVMKFLNLTSEKVSQEKVTEMMKKFCAQ
PWEEIKTSYAGVKEKYLSEYCFSGTYILSLLLQGYHFTADSWEHIHFIG
KIQGSDAGWTLGYMLNLTNMIPAEQPLSTPLSHSTYVFLMVLFSLVLFT
VAIIGLLIFHKPSYFWKDMV

The full length CD39 protein is encoded by the nucleic acid molecule according to SEQ ID NO: 10 below:

| | |
|---|---|
| 1 | atggaagata caaaggagtc taacgtgaag acattttgct ccaagaatat cctagccatc |
| 61 | cttggcttct cctctatcat agctgtgata gctttgcttg ctgtggggtt gacccagaac |
| 121 | aaagcattgc cagaaaacgt taagtatggg attgtgctgg atgcgggttc ttctcacaca |
| 181 | agtttataca tctataagtg gccagcagaa aaggagaatg acacaggcgt ggtgcatcaa |
| 241 | gtagaagaat gcagggttaa aggtcctgga atctcaaaat ttgttcagaa agtaaatgaa |
| 301 | ataggcattt acctgactga ttgcatggaa agagctaggg aagtgattcc aaggtcccag |
| 361 | caccaagaga cacccgttta cctgggagcc acggcaggca tgcggttgct caggatggaa |
| 421 | agtgaagagt tggcagacag ggttctggat gtggtggaga ggagcctcag caactacccc |
| 481 | tttgacttcc agggtgccag gatcattact ggccaagagg aaggtgccta tggctggatt |
| 541 | actatcaact atctgctggg caaattcagt cagaaaacaa ggtggttcag catagtccca |
| 601 | tatgaaacca ataatcagga aacctttgga gctttggacc ttgggggagc ctctacacaa |
| 661 | gtcacttttg taccccaaaa ccagactatc gagtccccag ataatgctct gcaatttcgc |
| 721 | ctctatggca aggactacaa tgtctacaca catagcttct tgtgctatgg gaaggatcag |
| 781 | gcactctggc agaaactggc caaggacatt caggttgcaa gtaatgaaat tctcagggac |
| 841 | ccatgctttc atcctggata taagaaggta gtgaacgtaa gtgaccttta caagaccccc |
| 901 | tgcaccaaga gatttgagat gactcttcca ttccagcagt ttgaaatcca gggtattgga |
| 961 | aactatcaac aatgccatca aagcatcctg gagctcttca acaccagtta ctgcccttac |
| 1021 | tcccagtgtg ccttcaatgg gattttcttg ccaccactcc agggggattt tggggcattt |
| 1081 | tcagcttttt actttgtgat gaagttttta aacttgacat cagagaaagt ctctcaggaa |
| 1141 | aaggtgactg agatgatgaa aaagttctgt gctcagcctt gggaggagat aaaaacatct |
| 1201 | tacgctggag taaaggagaa gtacctgagt gaatactgct tttctggtac ctacattctc |
| 1261 | tccctccttc tgcaaggcta tcatttcaca gctgattcct gggagcacat ccatttcatt |
| 1321 | ggcaagatcc agggcagcga cgccggctgg actttgggct acatgctgaa cctgaccaac |
| 1381 | atgatcccag ctgagcaacc attgtccaca cctctctccc actccaccta tgtcttcctc |
| 1441 | atggttctat tctccctggt ccttttcaca gtggccatca taggcttgct tatctttcac |
| 1501 | aagccttcat atttctggaa agatatggta tag |

The protein and encoding nucleic acid molecule are disclosed in Genbank Accessions NP_001767 and NM_001776.6, each of which is hereby incorporated by reference in its entirety.

A number of CD39 homologs exist in other species and can be used to practice the present invention. These include, without limitation, chimpanzee (>99% sequence identity to human, Genbank Accession PNI82205); orangutan (>98% sequence identity to human, Genbank Accession XP_009243927); macaque (>98% sequence identity to human, Genbank Accession XP_015311944); rhesus monkey (>98% sequence identity to human, Genbank Accession AFE66135); gorilla (>99% sequence identity to human, Genbank Accession XP_018890606); horse (>77% sequence identity to human, Genbank Accession XP_046510266); dog (>76% sequence identity to human, Genbank Accession XP_038296024); cow (>70% sequence identity to human, Genbank Accession NP_776961); cat (>79% sequence identity to human, Genbank Accession XP_023096552); and mouse (>75% sequence identity to human, Genbank Accession NP_001291650). Each of the above-identified Genbank Accessions is hereby incorporated by reference in its entirety.

Based on the foregoing, it is contemplated that variations of the human CD39 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 9 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human CD39 protein can also be tolerated if activity of the protein is not severely compromised.

One exemplary human CD73 protein comprises the amino acid sequence according to SEQ ID NO: 11 shown below:

MCPRAARAPATLLLALGAVLWPAAGAWELTILHTNDVHSRLEQTSEDSS
KCVNASRCMGGVARLFTKVQQIRRAEPNVLLLDAGDQYQGTIWFTVYKG
AEVAHFMNALRYDAMALGNHEFDNGVEGLIEPLLKEAKFPILSANIKAK
GPLASQISGLYLPYKVLPVGDEVVGIVGYTSKETPFLSNPGTNLVFEDE
ITALQPEVDKLKTLNVNKIIALGHSGFEMDKLIAQKVRGVDVVVGGHSN
TFLYTGNPPSKEVPAGKYPFIVTSDDGRKVPVVQAYAFGKYLGYLKIEF
DERGNVISSHGNPILLNSSIPEDPSIKADINKWRIKLDNYSTQELGKTI
VYLDGSSQSCRFRECNMGNLICDAMINNNLRHADETFWNHVSMCILNGG
GIRSPIDERNNGTITWENLAAVLPFGGTFDLVQLKGSTLKKAFEHSVHR
YGQSTGEFLQVGGIHVVYDLSRKPGDRVVKLDVLCTKCRVPSYDPLKMD
EVYKVILPNFLANGGDGFQMIKDELLRHDSGDQDINVVSTYISKMKVIY
PAVEGRIKFSTGSHCHGSFSLIFLSLWAVIFVLYQ

This human CD73 protein includes a signal sequence at amino acids 1-26, and the mature protein spans amino acids 27-549 following cleavage of the proprotein amino acids 550-574. The full length CD73 protein is encoded by the nucleic acid molecule according to SEQ ID NO: 12 below:

| | |
|---|---|
| 1 | atgtgtcccc gagccgcgcg ggcgcccgcg acgctactcc tcgccctggg cgcggtgctg |
| 61 | tggcctgcgg ctggcgcctg ggagcttacg attttgcaca ccaacgacgt gcacagccgg |
| 121 | ctggagcaga ccagcgagga ctccagcaag tgcgtcaacg ccagccgctg catgggtggc |
| 181 | gtggctcggc tcttcaccaa ggttcagcag atccgccgcg ccgaacccaa cgtgctgctg |
| 241 | ctggacgccg gcgaccagta ccagggcact atctggttca ccgtgtacaa gggcgccgag |
| 301 | gtggcgcact tcatgaacgc cctgcgctac gatgccatgg cactgggaaa tcatgaattt |
| 361 | gataatggtg tggaaggact gatcgagcca ctcctcaaag aggccaaatt tccaattctg |
| 421 | agtgcaaaca ttaaagcaaa ggggccacta gcatctcaaa tatcaggact ttatttgcca |
| 481 | tataaagttc ttcctgttgg tgatgaagtt gtgggaatcg ttggatacac ttccaaagaa |
| 541 | accccttttc tctcaaatcc agggacaaat ttagtgtttg aagatgaaat cactgcatta |
| 601 | caacctgaag tagataagtt aaaaactcta aatgtgaaca aaattattgc actgggacat |
| 661 | tcgggttttg aaatggataa actcatcgct cagaaagtga ggggtgtgga cgtcgtggtg |
| 721 | ggaggacact ccaacacatt tctttacaca ggcaatccac cttccaaaga ggtgcctgct |
| 781 | gggaagtacc cattcatagt cacttctgat gatgggcgga aggttcctgt agtccaggcc |
| 841 | tatgcttttg gcaaatacct aggctatctg aagatcgagt ttgatgaaag aggaaacgtc |
| 901 | atctcttccc atggaaatcc cattcttcta aacagcagca ttcctgaaga tccaagcata |
| 961 | aaagcagaca ttaacaaatg gaggataaaa ttggataatt attctaccca ggaattaggg |
| 1021 | aaaacaattg tctatctgga tggctcctct caatcatgcc gctttagaga atgcaacatg |
| 1081 | ggcaacctga tttgtgatgc aatgattaac aacaacctga gacacacgga tgaaatgttc |
| 1141 | tggaaccacg tatccatgtg cattttaaat ggaggtggta tccggtcgcc cattgatgaa |
| 1201 | cgcaacaatg gcacaattac ctgggagaac ctggctgctg tattgccctt tggaggcaca |
| 1261 | tttgacctag tccagttaaa aggttccacc ctgaagaagg cctttgagca tagcgtgcac |
| 1321 | cgctacggcc agtccactgg agagttcctg caggtgggcg gaatccatgt ggtgtatgat |
| 1381 | ctttcccgaa aacctggaga cagagtagtc aaattagatg ttctttgcac caagtgtcga |
| 1441 | gtgcccagtt atgaccctct caaaatggac gaggtatata aggtgatcct cccaaacttc |
| 1501 | ctggccaatg gtggagatgg gttccagatg ataaaagatg aattattaag acatgactct |
| 1561 | ggtgaccaag atatcaacgt ggtttctaca tatatctcca aaatgaaagt aatttatcca |
| 1621 | gcagttgaag gtcggatcaa gttttccaca ggaagtcact gccatggaag cttttcttta |
| 1681 | atatttcttt cactttgggc agtgatcttt gttttatacc aatag |

The protein and encoding nucleic acid molecule are disclosed in Genbank Accessions AAH65937 and BC065937.1, each of which is hereby incorporated by reference in its entirety.

A number of CD73 homologs exist in other species and can be used to practice the present invention. These include, without limitation, chimpanzee (>99% sequence identity to human, Genbank Accession JAA33084); orangutan (>99% sequence identity to human, Genbank Accession XP_002817156); macaque (>98% sequence identity to human, Genbank Accession EHH53214); rhesus monkey (>98% sequence identity to human, Genbank Accession XP_001086989); gorilla (>99% sequence identity to human, Genbank Accession XP_004044409); horse (>90% sequence identity to human, Genbank Accession XP_023506526); dog (>90% sequence identity to human, Genbank Accession XP_038540011); cow (>89% sequence identity to human, Genbank Accession AAI14094); cat (>92% sequence identity to human, Genbank Accession XP_011280799); and mouse (>86% sequence identity to human, Genbank Accession AAI19269). Each of the above-identified Genbank Accessions is hereby incorporated by reference in its entirety.

Based on the foregoing, it is contemplated that variations of the human CD73 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 11 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human CD73 protein can also be tolerated if activity of the protein is not severely compromised.

IL-10 is a key anti-inflammatory mediator ensuring protection of a host from over-exuberant responses to pathogens and microbiota, while playing important roles in other settings as sterile wound healing, autoimmunity, cancer, and homeostasis. A wealth of data demonstrates that the IL-10/STAT3 axis as a major transcriptional inhibitor of genes encoding cytokines, chemokines, cell-surface molecules, and other molecules required for a full immune response. Therefore, IL-10 is a broad-spectrum immunosuppressant that suppresses macrophages, dendritic cells (DCs), and T cells. These features make IL-10 an ideal candidate for prevention of allorejection.

One exemplary human IL-10 protein comprises the amino acid sequence according to SEQ ID NO: 13 shown below:

MHSSALLCCLVLLTGVRASPGQGTQSENSCTHfPGNLPNMLRDLRDAFS
RVKTFFQMKDQLDNLLLKESLLEDFKGYLGCQALSEMIQFYLEEVMPQA
ENQDPDIKAHVNSLGENLKTLRLRLRRCHRFLPCENKSKAVEQVKNAFN
KLQEKGIYKAMSEFDIFINYIEAYMTMKIRN

This human IL-10 protein includes a signal sequence at amino acids 1-18, and the mature protein spans amino acids 19-178. The full-length IL-10 protein is encoded by the nucleic acid molecule according to SEQ ID NO: 14 below:

| | |
|---|---|
| 1 | atgcacagct cagcactgct ctgttgcctg gtcctcctga ctggggtgag ggccagccca |
| 61 | ggccagggca cccagtctga gaacagctgc acccacttcc caggcaacct gcctaacatg |
| 121 | cttcgagatc tccgagatgc cttcagcaga gtgaagactt totttcaaat gaaggatcag |
| 181 | ctggacaact tgttgttaaa ggagtccttg ctggaggact ttaagggtta cctgggttgc |
| 241 | caagccttgt ctgagatgat ccagttttac ctggaggagg tgatgcccca agctgagaac |
| 301 | caagacccag acatcaaggc gcatgtgaac tccctggggg agaacctgaa gaccctcagg |
| 361 | ctgaggctac ggcgctgtca tcgatttett ccctgtgaaa acaagagcaa ggccgtggag |
| 421 | caggtgaaga atgcctttaa taagctccaa gagaaaggca tctacaaagc catgagtgag |
| 481 | tttgacatct tcatcaacta catagaagcc tacatgacaa tgaagatacg aaactga |

The protein and encoding nucleic acid molecule are disclosed in Genbank Accessions NP_000563 and NM_000572, each of which is hereby incorporated by reference in its entirety.

A number of IL-10 homologs exist in other species and can be used to practice the present invention. These include, without limitation, chimpanzee (>99% sequence identity to human, Genbank Accession NP_001129092); orangutan (>97% sequence identity to human, Genbank Accession XP_002809587); macaque (>96% sequence identity to human, Genbank Accession P79338); rhesus monkey (>95% sequence identity to human, Genbank Accession NP_001038192); gorilla (>98% sequence identity to human, Genbank Accession XP_004028338); horse (>84% sequence identity to human, Genbank Accession AFI70769); dog (>76% sequence identity to human, Genbank Accession P48411); cow (>77% sequence identity to human, Genbank Accession P43480); cat (>79% sequence identity to human, Genbank Accession P55029); and mouse (>73% sequence identity to human, Genbank Accession NP_034678). Each of the above-identified Genbank Accessions is hereby incorporated by reference in its entirety.

Based on the foregoing, it is contemplated that variations of the human IL-10 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 13 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human IL-10 protein can also be tolerated if activity of the protein is not severely compromised.

Indoleamine 2,3-dioxygenase 1 (IDO1) has been shown to be constitutively expressed in most human tumors, and IDO1 has been shown in mice to prevent tumor cell rejection by preimmunized mice. This effect is accompanied by a lack of accumulation of specific T cells at the tumor site and can be partly reverted by systemic treatment of mice with an inhibitor of IDO1. Therefore, IDO1 is an immunosuppressant that suppresses T cells, which makes IDO1 an ideal candidate for prevention of allorejection.

One exemplary human IDO1 protein comprises the amino acid sequence according to SEQ ID NO: 15 shown below:

MAHAMENSWTISKEYHIDEEVGFALPNPQENLPDFYNDWMFIAKHLPDL
IESGQLRERVEKLNMLSIDHLTDHKSQRLARLVLGCITMAYVWGKGHGD
VRKVLPRNIAVPYCQLSKKLELPPILVYADCVLANWKKKDPNKPLTYEN
MDVLFSFRDGDCSKGFFLVSLLVEIAAASAIKVIPTVFKAMQMQERDTL
LKALLEIASCLEKALQVFHQIHDHVNPKAFFSVLRIYLSGWKGNPQLSD
GLVYEGFWEDPKEFAGGSAGQSSVFQCFDVLLGIQQTAGGGHAAQFLQD
MRRYMPPAHRNFLCSLESNPSVREFVLSKGDAGLREAYDACVKALVSLR
SYHLQIVTKYILIPASQQPKENKTSEDPSKLEAKGTGGTDLMNFLKTVR
STTEKSLLKEG

The full length IDO1 protein is encoded by the nucleic acid molecule according to SEQ ID NO: 16 below:

| | |
|---|---|
| 1 | atggcacacg ctatggaaaa ctcctggaca atcagtaaag agtaccatat tgatgaagaa |
| 61 | gtgggctttg ctctgccaaa tccacaggaa aatctacctg atttttataa tgactggatg |
| 121 | ttcattgcta aacatctgcc tgatctcata gagtctggcc agcttcgaga aagagttgag |
| 181 | aagttaaaca tgctcagcat tgatcatctc acagaccaca agtcacagcg ccttgcacgt |
| 241 | ctagttctgg gatgcatcac catggcatat gtgtggggca aaggtcatgg agatgtccgt |
| 301 | aaggtcttgc caagaaatat tgctgttcct tactgccaac tctccaagaa actggaactg |
| 361 | cctcctattt tggtttatgc agactgtgtc ttggcaaact ggaagaaaaa ggatcctaat |
| 421 | aagcccctga cttatgagaa catggacgtt ttgttctcat ttcgtgatgg agactgcagt |
| 481 | aaaggattct tcctggtctc tctattggtg gaaatagcag ctgcttctgc aatcaaagta |
| 541 | attcctactg tattcaaggc aatgcaaatg caagaacggg acactttgct aaaggcgctg |
| 601 | ttggaaatag cttcttgctt ggagaaagcc cttcaagtgt ttcaccaaat ccacgatcat |
| 661 | gtgaacccaa aagcattttt cagtgttctt cgcatatatt tgtctggctg gaaaggcaac |
| 721 | ccccagctat cagacggtct ggtgtatgaa gggttctggg aagacccaaa ggagtttgca |
| 781 | gggggcagtg caggccaaag cagcgtcttt cagtgctttg acgtcctgct gggcatccag |
| 841 | cagactgctg gtggaggaca tgctgctcag ttcctccagg acatgagaag atatatgcca |
| 901 | ccagctcaca ggaacttcct gtgctcatta gagtcaaatc cctcagtccg tgagtttgtc |
| 961 | ctttcaaaag gtgatgctgg cctgcgggaa gcttatgacg cctgtgtgaa agctctggtc |
| 1021 | tccctgagga gctaccatct gcaaatcgtg actaagtaca tcctgattcc tgcaagccag |
| 1081 | cagccaaagg agaataagac ctctgaagac ccttcaaaac tggaagccaa aggaactgga |
| 1141 | ggcactgatt taatgaattt cctgaagact gtaagaagta caactgagaa atcccttttg |
| 1201 | aaggaaggtt aa |

The protein and encoding nucleic acid molecule are disclosed in Genbank Accessions NP_002155 and NM_002164, each of which is hereby incorporated by reference in its entirety.

A number of IDO1 homologs exist in other species and can be used to practice the present invention. These include, without limitation, chimpanzee (>99% sequence identity to human, Genbank Accession XP_001137531); orangutan (>98% sequence identity to human, Genbank Accession XP_024106901); macaque (>93% sequence identity to human, Genbank Accession XP_005563203); rhesus monkey (>93% sequence identity to human, Genbank Accession NP_001070951); gorilla (>99% sequence identity to human, Genbank Accession XP_004046983); horse (>72% sequence identity to human, Genbank Accession XP_014592024); dog (>68% sequence identity to human, Genbank Accession XP_038545722); cow (>68% sequence identity to human, Genbank Accession NP_001095336); cat (>68% sequence identity to human, Genbank Accession XP_038545722); and mouse (>62% sequence identity to human, Genbank Accession NP_032350). Each of the above-identified Genbank Accessions is hereby incorporated by reference in its entirety.

Based on the foregoing, it is contemplated that variations of the human IDO1 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 15 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human IDO1 protein can also be tolerated if activity of the protein is not severely compromised.

Galectin-9 (LGALS9) is a tandem protein that contains two ligand-binding domains fused together by a peptide linker. Although LGALS9 lacks a secretory domain, it is believed to be trafficked onto the cell surface by T cell immunoglobulin and mucin domain containing protein 3 (Tim-3). Once on the cell surface, proteolytic shedding results in the release of a soluble form of both Tim-3 and LGALS9. Both Tim-3 and LGALS9 act to suppress anti-cancer immune surveillance. Secreted LGALS9 contributes to anti-cancer immune suppression by killing cytotoxic T lymphocytes and impairing the activity of natural killer cells to allow for disease progression. Therefore, LGALS9 is an immunosuppressant that suppresses T cells, which makes LGALS9 an ideal candidate for prevention of allorejection.

One exemplary human LGALS9 protein comprises the amino acid sequence according to SEQ ID NO: 40 shown below:

MAFSGSQAPYLSPAVPFSGTIQGGLQDGLQITVNGTVLSSSGTRFAVNF
QTGFSGNDIAFHENPRFEDGGYVVCNTRQNGSWGPEERKTHMPFQKGMP
FDLCFLVQSSDFKVMVNGILFVQYFHRVPFHRVDTISVNGSVQLSYISF
QNPRTVPVQPAFSTVPFSQPVCFPPRPRGRRQKPPGVWPANPAPITQTV
IHTVQSAPGQMESTPAIPPMMYPHPAYPMPFITTILGGLYPSKSILLSG
TVLPSAQRFHINLCSGNHIAFHLNPRFDENAVVRNTQIDNSWGSEERSL
PRKMPFVRGQSFSVWILCEAHCLKVAVDGQHLFEYYHRLRNLPTINRLE
VGGDIQLTHVQT

The full length LGALS9 protein is encoded by the nucleic acid molecule according to SEQ ID NO: 41 below:

| | |
|---|---|
| 1 | atggccttca gcggttccca ggctccctac ctgagtccag ctgtcccctt ttctgggact |
| 61 | attcaaggag gtctccagga cggacttcag atcactgtca atgggaccgt tctcagctcc |
| 121 | agtggaacca ggtttgctgt gaactttcag actggcttca gtggaaatga cattgccttc |
| 181 | cacttcaacc ctcggtttga agatggaggg tacgtggtgt gcaacacgag gcagaacgga |
| 241 | agctgggggc ccgaggagag gaagacacac atgcctttcc agaaggggat gccctttgac |
| 301 | ctctgcttcc tggtgcagag ctcagatttc aaggtgatgg tgaacgggat cctcttcgtg |
| 361 | cagtacttcc accgcgtgcc cttccaccgt gtggacacca tctccgtcaa tggctctgtg |
| 421 | cagctgtcct acatcagctt ccagaacccc cgcacagtcc ctgttcagcc tgccttctcc |
| 481 | acggtgccgt tctcccagcc tgtctgtttc ccacccaggc ccagggggcg cagacaaaaa |
| 541 | cctcccggcg tgtggcctgc caacccggct cccattaccc agacagtcat ccacacagtg |
| 601 | cagagcgccc ctggacagat gttctctact cccgccatcc cacctatgat gtacccccac |
| 661 | cccgcctatc cgatgccttt catcaccacc attctgggag ggctgtaccc atccaagtcc |
| 721 | atcctcctgt caggcactgt cctgcccagt gctcagaggt tccacatcaa cctgtgctct |
| 781 | gggaaccaca tcgccttcca cctgaacccc cgttttgatg agaatgctgt ggtccgcaac |
| 841 | acccagatcg acaactcctg ggggtctgag gagcgaagtc tgccccgaaa aatgcccttc |
| 901 | gtccgtggcc agagcttctc agtgtggatc ttgtgtgaag ctcactgcct caaggtggcc |
| 961 | gtggatggtc agcacctgtt tgaatactac catcgcctga ggaacctgcc caccatcaac |
| 1021 | agactggaag tggggggcga catccagctg acccatgtgc agacatag |

The protein and encoding nucleic acid molecule are disclosed in Genbank Accessions NP_033665 and NM_009587, each of which is hereby incorporated by reference in its entirety.

A number of LGALS9 homologs exist in other species and can be used to practice the present invention. These include, without limitation, chimpanzee (>95% sequence identity to human, Genbank Accession XP_024206173); orangutan (>96% sequence identity to human, Genbank Accession PNJ34131); macaque (>80% sequence identity to human, Genbank Accession XP_045231137); rhesus monkey (>94% sequence identity to human, Genbank Accession XP_014974370); gorilla (>98% sequence identity to human, Genbank Accession XP_004042123); horse (>61% sequence identity to human, Genbank Accession XP_001918023); dog (>74% sequence identity to human, Genbank Accession XP_038530897); cow (>74% sequence identity to human, Genbank Accession DAA19125); cat (>76% sequence identity to human, Genbank Accession XP_003996571); and mouse (>69% sequence identity to human, Genbank Accession NP_034838). Each of the above-identified Genbank Accessions is hereby incorporated by reference in its entirety.

Based on the foregoing, it is contemplated that variations of the human LGALS9 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 40 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human LGALS9 protein can also be tolerated if activity of the protein is not severely compromised.

CD155 is overexpressed in the tumor microenvironment of various cancers. CD155 expression has been shown to be coregulated with PD-L1 on tumor-associated macrophages, and transcriptionally regulated by persistently active aryl hydrocarbon receptor (AhR) (see McKay et al., “Aryl Hydrocarbon Receptor Signaling Controls CD155 Expression on Macrophages and Mediates Tumor Immunosuppression,” J. Immunol. 206(6):1385-1394 (2021), which is hereby incorporated by reference in its entirety). McKay et al. also showed that therapeutic inhibition of AhR reversed tumor immunosuppression in an immune competent murine tumor model. Thus, CD155 is an immunosuppressant that suppresses T cells, which makes CD155 an ideal candidate for prevention of allorejection.

One exemplary human CD155 protein comprises the amino acid sequence according to SEQ ID NO: 42 shown below:

MARAMAAAWPLLLVALLVLSWPPPGTGDVVVQAPTQVPGFLGDSVTLPC
YLQVPNMEVTHVSQLTWARHGESGSMAVFHQTQGPSYSESKRLEFVAAR
LGAELRNASLRMFGLRVEDEGNYTCLFVTFPQGSRSVDIWLRVLAKPQN
TAEVQKVQLTGEPVPMARCVSTGGRPPAQITWHSDLGGMPNTSQVPGFL
SGTVTVTSLWILVPSSQVDGKNVTCKVEHESFEKPQLLTVNLTVYYPPE
VSISGYDNNWYLGQNEATLTCDARSNPEPTGYNWSTTMGPLPPFAVAQG
AQLLIRPVDKPINTTLICNVTNALGARQAELTVQVKEGPPSEHSGMSRN
AIIFLVLGILVFLILLGIGIYFYWSKCSREVLWHCHLCPSSTEHASASA
NGHVSYSAVSRENSSSQDPQTEGTR

The full length CD155 protein is encoded by the nucleic acid molecule according to SEQ ID NO: 43 below:

| | |
|---|---|
| 1 | atggcccgag ccatggccgc cgcgtggccg ctgctgctgg tggcgctact ggtgctgtcc |
| 61 | tggccacccc caggaaccgg ggacgtcgtc gtgcaggcgc ccacccaggt gcccggcttc |
| 121 | ttgggcgact ccgtgacgct gccctgctac ctacaggtgc ccaacatgga ggtgacgcat |
| 181 | gtgtcacagc tgacttgggc gcggcatggt gaatctggca gcatggccgt cttccaccaa |
| 241 | acgcagggcc ccagctattc ggagtccaaa cggctggaat tcgtggcagc cagactgggc |
| 301 | gcggagctgc ggaatgcctc gctgaggatg ttcgggttgc gcgtagagga tgaaggcaac |
| 361 | tacacctgcc tgttcgtcac gttcccgcag ggcagcagga gcgtggatat ctggctccga |
| 421 | gtgcttgcca agccccagaa cacagctgag gttcagaagg tccagctcac tggagagcca |
| 481 | gtgcccatgg cccgctgcgt ctccacaggg ggtcgcccgc cagcccaaat cacctggcac |
| 541 | tcagacctgg gcgggatgcc caatacgagc caggtgccag ggttcctgtc tggcacagtc |
| 601 | actgtcacca gcctctggat attggtgccc tcaagccagg tggacggcaa gaatgtgacc |
| 661 | tgcaaggtgg agcacgagag ctttgagaag cctcagctgc tgactgtgaa cctcaccgtg |
| 721 | tactaccccc cagaggtatc catctctggc tatgataaca actggtacct tggccagaat |
| 781 | gaggccaccc tgacctgcga tgctcgcage aacccagagc ccacaggcta taattggagc |
| 841 | acgaccatgg gtcccctgcc accctttgct gtggcccagg gcgcccagct cctgatccgt |
| 901 | cctgtggaca aaccaatcaa cacaacttta atctgcaacg tcaccaatgc cctaggagct |
| 961 | cgccaggcag aactgaccgt ccaggtcaaa gagggacctc ccagtgagca ctcaggcatg |
| 1021 | tcccgtaacg ccatcatctt cctggttctg ggaatcctgg tttttctgat cctgctgggg |
| 1081 | atcgggattt atttctattg gtccaaatgt tcccgtgagg tcctttggca ctgtcatctg |
| 1141 | tgtccctcga gtacagagca tgccagcgcc tcagctaatg ggcatgtctc ctattcagct |
| 1201 | gtgagcagag agaacagctc ttcccaggat ccacagacag agggcacaag gtga |

The protein and encoding nucleic acid molecule are disclosed in Genbank Accessions NP_006496 and NM_006505, each of which is hereby incorporated by reference in its entirety.

A number of CD155 homologs exist in other species and can be used to practice the present invention. These include, without limitation, chimpanzee (>97% sequence identity to human, Genbank Accession XP_001161582); orangutan (>96% sequence identity to human, Genbank Accession XP_024093654); macaque (>90% sequence identity to human, Genbank Accession XP_045234933); rhesus monkey (>90% sequence identity to human, Genbank Accession NP_001036851); horse (>52% sequence identity to human, Genbank Accession XP_023505635); dog (>56% sequence identity to human, Genbank Accession XP_038512643); cow (>54% sequence identity to human, Genbank Accession XP_005219484); cat (>62% sequence identity to human, Genbank Accession XP_044901841); and mouse (>43% sequence identity to human, Genbank Accession NP_081790). Each of the above-identified Genbank Accessions is hereby incorporated by reference in its entirety.

Based on the foregoing, it is contemplated that variations of the human CD155 protein can also be used to practice the present invention, including those that have at least 40% sequence identity over the full length thereof, including at least 45% sequence identity, at least 50% sequence identity, at least 55% sequence identity, at least 60% sequence identity, at least 65% sequence identity, at least 70% sequence identity, at least 75% sequence identity, at least 80% sequence identity, at least 85% sequence identity, at least 90% sequence identity to SEQ ID NO: 42 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human CD155 protein can also be tolerated if activity of the protein is not severely compromised.

Arginase 1 (Arg1) degrades L-arginine and its expression is substantially increased in cancer. Increased activity of Arg1 correlates with more advanced disease and worse clinical prognosis. Nearly all types of myeloid cells produce arginases and the increased number of myeloid-derived suppressor cells and macrophages correlates with inferior clinical outcomes. Arg1 is involved in the immune escape in the tumor microenvironment. Therefore, Arg1 is an immunosuppressant that suppresses T cells, which makes Arg1 an ideal candidate for prevention of allorejection.

One exemplary human Arg1 protein comprises the amino acid sequence according to SEQ ID NO: 44 shown below:

MSAKSRTIGIIGAPFSKGQPRGGVEEGPTVLRKAGLLEKLKEQVTQNFL
ILECDVKDYGDLPFADIPNDSPFQIVKNPRSVGKASEQLAGKVAEVKKN
GRISLVLGGDHSLAIGSISGHARVHPDLGVIWVDAHTDINTPLTTTSGN
LHGQPVSFLLKELKGKIPDVPGFSWVTPCISAKDIVYIGLRDVDPGEHY
ILKTLGIKYFSMTEVDRLGIGKVMEETLSYLLGRKKRPIHLSFDVDGLD
PSFTPATGTPVVGGLTYREGLYITEEIYKTGLLSGLDIMEVNPSLGKTP
EEVTRTVNTAVAITLACFGLAREGNHKPIDYLNPPK

The full length Arg1 protein is encoded by the nucleic acid molecule according to SEQ ID NO: 45 below:

| | |
|---|---|
| 1 | atgagcgcca agtccagaac catagggatt attggagctc ctttctcaaa gggacagcca |
| 61 | cgaggagggg tggaagaagg ccctacagta ttgagaaagg ctggtctgct tgagaaactt |
| 121 | aaagaacaag taactcaaaa ctttttaatt ttagagtgtg atgtgaagga ttatggggac |
| 181 | ctgccctttg ctgacatccc taatgacagt ccctttcaaa ttgtgaagaa tccaaggtct |
| 241 | gtgggaaaag caagcgagca gctggctggc aaggtggcag aagtcaagaa gaacggaaga |
| 301 | atcagcctgg tgctgggcgg agaccacagt ttggcaattg gaagcatctc tggccatgcc |
| 361 | agggtccacc ctgatcttgg agtcatctgg gtggatgctc acactgatat caacactcca |
| 421 | ctgacaacca caagtggaaa cttgcatgga caacctgtat ctttcctcct gaaggaacta |
| 481 | aaaggaaaga ttcccgatgt gccaggattc tcctgggtga ctccctgtat atctgccaag |
| 541 | gatattgtgt atattggctt gagagacgtg gaccctgggg aacactacat tttgaaaact |
| 601 | ctaggcatta aatacttttc aatgactgaa gtggacagac taggaattgg caaggtgatg |
| 661 | gaagaaacac tcagctatct actaggaaga aagaaaaggc caattcatct aagttttgat |
| 721 | gttgacggac tggacccatc tttcacacca gctactggca caccagtcgt gggaggtctg |
| 781 | acatacagag aaggtctcta catcacagaa gaaatctaca aaacagggct actctcagga |
| 841 | ttagatataa tggaagtgaa cccatccctg gggaagacac cagaagaagt aactcgaaca |
| 901 | gtgaacacag cagttgcaat aaccttggct tgtttcggac ttgctcggga gggtaatcac |
| 961 | aagcctattg actaccttaa cccacctaag taa |

The protein and encoding nucleic acid molecule are disclosed in Genbank Accessions NP_001231367 and NM_001244438, each of which is hereby incorporated by reference in its entirety.

A number of Arg1 homologs exist in other species and can be used to practice the present invention. These include, without limitation, chimpanzee (>99% sequence identity to human, Genbank Accession PNI87226); orangutan (>98% sequence identity to human, Genbank Accession PNJ78548); macaque (>95% sequence identity to human, Genbank Accession XP_005551900); rhesus monkey (>95% sequence identity to human, Genbank Accession XP_001103609); gorilla (>96% sequence identity to human, Genbank Accession XP_004044730); horse (>89% sequence identity to human, Genbank Accession XP_001503335); dog (>90% sequence identity to human, Genbank Accession XP_038538818); cow (>88% sequence identity to human, Genbank Accession NP_001039619); cat (>91% sequence identity to human, Genbank Accession XP_003986598); and mouse (>84% sequence identity to human, Genbank Accession NP_031508). Each of the above-identified Genbank Accessions is hereby incorporated by reference in its entirety.

Based on the foregoing, it is contemplated that variations of the human Arg1 protein can also be used to practice the present invention, including those that have at least 60% sequence identity over the full length thereof, including at least 65% sequence identity, at least 70% sequence identity, at least 75% sequence identity, at least 80% sequence identity, at least 85% sequence identity, at least 90% sequence identity to SEQ ID NO: 44 (such as at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity). Amino acids that are generally tolerant to change and can be replaced with conserved or non-conserved amino acids, whereas amino acids that are intolerant to non-conserved amino acid changes can be replaced with conserved amino acids. Likewise, truncations of N-terminal and C-terminal regions of the human Arg1 protein can also be tolerated if activity of the protein is not severely compromised.

According to one embodiment, a combination of the immunomodulatory proteins or polypeptides includes a PD-L1 polypeptide and a CTLA4-Ig fusion protein. In preferred embodiments, the recombinant mesenchymal stromal cell substantially overexpresses both PD-L1 and CTLA4-Ig (when compared to a non-recombinant mesenchymal stromal cell). In these embodiments, the PD-L1 is cell surfaced expressed on the eMSCs and the CTLA4-Ig fusion protein is secreted by the eMSCs into the local environment (i.e., of the mixed cell population).

According to one embodiment, a combination of the immunomodulatory proteins or polypeptides includes any two or more of CD47, CD39, CD73, IL-10, and IDO1. In certain embodiments, the combination of immunomodulatory proteins or polypeptides includes CD47, CD39, CD73, and IL-10. In certain embodiments, the combination of immunomodulatory proteins or polypeptides includes CD39, CD73, IDO1, and IL-10. In preferred embodiments, the recombinant mesenchymal stromal cell substantially overexpresses each of the combination of immunomodulatory proteins or polypeptides (when compared to a non-recombinant mesenchymal stromal cell). In these embodiments, the CD47, CD39, and/or CD73 are cell surfaced expressed on the eMSCs and the IL-10 and/or IDO1 is secreted by the eMSCs into the local environment (i.e., of the mixed cell population).

The construction of the recombinant MSCs (i.e., eMSCs) described herein may be carried out using techniques known to a person skilled in the art.

In one embodiment, the recombinant MSCs as described herein are characterized in that the exogenous nucleic acid comprises viral vector sequences, for example in the form of a viral expression construct.

In one embodiment, the recombinant MSCs as described herein are characterized in that the exogenous nucleic acid is a non-viral expression construct.

As used herein, “nucleic acid” shall mean any nucleic acid molecule, including, without limitation, DNA, RNA and hybrids or modified variants thereof. An “exogenous nucleic acid” or “exogenous genetic element” relates to any nucleic acid introduced into the MSCs, which is not a component of the cells “original” or “natural” genome. Exogenous nucleic acids may be integrated or non-integrated in the genetic material of the MSCs, or may refer to stably transduced nucleic acids.

Where the exogenous nucleic acid comprises a transgene including a promoter, an opening reading frame encoding the one or more immunomodulatory proteins or polypeptides, and 3′ untranslated regions, the various components of the transgene can be ligated together using known materials and techniques.

Any given gene delivery method is encompassed by the invention and preferably relates to viral or non-viral vectors, as well as biological or chemical methods of transfection, or combinations thereof. The methods can yield either stable or transient gene expression in the system used.

Non-viral vectors include, without limitation, plasmid vectors and transposon-based vectors, or any other vector suitable for introduction of the exogenous gene construct described herein into the MSCs to facilitate the expression of the immunomodulatory protein or polypeptide.

Genetically modified viruses have been widely applied for the delivery of genes into MSCs. Exemplary viral vectors include, without limitation, vaccina vectors, lentiviral vector (integration competent or integration-defective lentiviral vectors), adenoviral vectors, adeno-associated viral vectors, and vectors for baculovirus expression.

Adenoviruses may be applied, or RNA viruses such as Lentiviruses, or other retroviruses. Adenoviruses have been used to generate a series of vectors for gene transfer in the field of gene therapy and cellular engineering. The initial generation of adenovirus vectors were produced by deleting the EI gene (required for viral replication) generating a vector with a 4 kb cloning capacity. An additional deletion of E3 (responsible for host immune response) allowed an 8 kb cloning capacity. Further generations have been produced encompassing E2 and/or E4 deletions. The use of any given adenovirus vector, for example those according to those described above, is encompassed by the present invention.

Lentiviruses are members of Retroviridae family of viruses (Scherr et al., “Gene Transfer into Hematopoietic Stem Cells Using Lentiviral Vectors,” Curr Gene Ther. 2(1):45-55 (2002), which is hereby incorporated by reference in its entirety. Lentivirus vectors are generated by deletion of the entire viral sequence with the exception of the LTRs and cis acting packaging signals. The resultant vectors have a cloning capacity of about 8 kb. One distinguishing feature of these vectors from retroviral vectors is their ability to transduce dividing and non-dividing cells as well as terminally differentiated cells.

Non-viral methods may also be employed, and these also include the application of targeted gene integration or modification through the use of nuclease-based gene editing, integrase or transposase technologies. These represent approaches for vector transformation that have the advantage of being both efficient, and often site-specific in their integration. Physical methods to introduce vectors into cells are known to a skilled person. One example relates to electroporation, which relies on the use of brief, high voltage electric pulses which create transient pores in the membrane by overcoming its capacitance. One advantage of this method is that it can be utilized for both stable and transient gene expression in most cell types. Alternative methods relate to the use of liposomes or protein transduction domains. Appropriate methods are known to a skilled person and are not intended as limiting embodiments of the present invention.

The invention encompasses the use of more than one virus, or a virus and other gene editing event or genetic modification, including the use of or mRNA or other genetic modification in order to manipulate gene expression.

In one embodiment the genetically modified MSC as described herein is characterized in that the promoter (with or without any enhancer elements) yields constitutive expression of the exogenous nucleic acid. Due to the need to create immunosuppressive local environments following the co-administration of the recombinant MSCs and islet cells (or islets), the use of a constitutive promoter for expression of the one or more immunomodulatory proteins or polypeptides is preferred.

Non-limiting examples of suitable promoters for use in the recombinant transgene, or for modifying the promoter region of a native gene encoding an immunomodulatory protein, include, the EF1 alpha promoter, for example the EF1alphaS promoter; the PGK promoter; the CMV or SV40 viral promoters; the GAG promoter; the UBC promoter. Other constitutive promoters can also be used (see Qin et al., “Systematic Comparison of Constitutive Promoters and the Doxycycline-Inducible Promoter,” PLoS One 5(5):e10611 (2010), which is hereby incorporated by reference in its entirety).

In some embodiments, the exogenous gene construct further comprises a selection marker. Suitable selection markers for mammalian cells are known in the art, and include for example, thymidine kinase, dihydrofolate reductase (together with methotrexate as a DHFR amplifier), aminoglycoside phosphotransferase, hygromycin B phosphotransferase, asparagine synthetase, adenosine deaminase, metallothionein, and antibiotic resistant genes, e.g., the puromycin resistance gene or the neomycin resistance gene.

In some embodiments, the exogenous gene construct further encodes at least one marker domain. Non-limiting examples of marker domains include fluorescent proteins, purification tags, and epitope tags. The marker domain may be operatively coupled to the constitutive mammalian promoter.

In some aspects, the marker domain may be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g, ECFP, Cerulean, CyPet, AmCyanl, Midoriishi-Cyan), red fluorescent proteins (mKate, mKate2, mPlum, DsRed monomer, mCherry, mRFPl, DsRed-Express, DsRed2, DsRed-Monomer, HcRed-Tandem, HcRedl, AsRed2, mRasberry, mStrawberry, Jred), and orange fluorescent proteins (mOrange, mKO, Kusabira-Orange, Monomeric Kusabira-Orange, mTangerine, tdTomato) or any other suitable fluorescent protein.

In other aspects, the marker domain may be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AUl, AUS, E, ECS, E2, FLAG, HA, nus, Softag 1, Softag 3, Strep, SBP, Glu-Glu, HSV, KT3, S, SI, T7, V5, VSV-G, 6×His, biotin carboxyl carrier protein (BCCP), and calmodulin.

In some embodiments, the recombinant genetic constructs as disclosed herein that promote expression of the one or more immunomodulatory proteins or polypeptides include a CRISPR/Cas9 system or zinc-finger nuclease.

CRISPR/CRISPR-associated (Cas) systems use single guide RNAs to target and cleave DNA elements in a sequence-specific manner. CRISPR/Cas systems are well known in the art and include, e.g., the type II CRISPR system from Streptococcus pyogenes (Qi et al, “Repurposing CRISPR as an RNA-Guided Platform for Sequence-Specific Control of Gene Expression,” Cell 152(5):1173-1183 (2013), which is hereby incorporated by reference in its entirety). The Streptococcuspyogenes type II CRISPR system includes a single gene encoding the Cas9 protein and two RNAs, a mature CRISPR RNA (crRNA), and a partially complementary trans-acting RNA (tracrRNA). Maturation of the crRNA requires tracrRNA and RNase II. However, this requirement can be by-passed by using an engineered small guide RNA (sgRNA) containing a designed hairpin that mimics the tracrRNA-crRNA complex. Base pairing between the sgRNA and target DNA causes double-strand breaks (DSBs) due to the endonuclease activity of Cas9. Binding specificity is determined by both sgRNA-DNA base pairing and a short DNA motif (protospacer adjacent motif (PAM) sequence: NGG) juxtaposed to the DNA complementary region.

In some embodiments, the CRISPR/Cas 9 system encoded by the recombinant genetic construct comprises a Cas9 protein and a sgRNA.

The Cas9 protein may comprise a wild-type Cas9 protein or a nuclease-deficient Cas9 protein. Binding of wild-type Cas9 to the sgRNA forms a protein-RNA complex that mediates cleavage of a target DNA by the cas9 nuclease. Binding of nuclease deficient Cas9 to the sgRNA forms a protein-RNA complex that mediates transcriptional regulation of a target DNA by the nuclease deficient Cas9 (Qi et al, “Repurposing CRISPR as an RNA-Guided Platform for Sequence-Specific Control of Gene Expression,” Cell 152(5):1173-1183 (2013); Maeder et al., “CRISPR RNA-Guided Activation of Endogenous Human Genes,” Nat. Methods 10(10):977-999 (2013); and Gilbert et al., “CRISPR-Mediated Modular RNA-Guided Regulation of Transcription in Eukaryotes,” Cell 154(2):442-451 (2013), each of which is hereby incorporated by reference in its entirety).

The sgRNA comprises a region complementary to a specific DNA sequence (e.g., a region of the 5′ untranslated region in a native/endogenous gene encoding one of the immunomodulatory proteins), a hairpin for Cas9 binding, and/or a transcription terminator (Qi et al., “Repurposing CRISPR as an RNA-Guided Platform for Sequence-Specific Control of Gene Expression,” Cell 152(5):1173-1183 (2013), which is hereby incorporated by reference in its entirety). Methods of designing sgRNA for the purposes of targeting specific gene sequence are well known in the art and are described in more detail in, e.g., WO2015/089364, WO2014/191521 and WO2015/065964, each of which is hereby incorporated by reference in its entirety).

In another embodiment, the one or more agents encoded by the recombinant genetic construct disclosed herein for purposes of modifying expression of native/endogenous genes encoding one of the immunomodulatory proteins is a zinc finger nuclease. Zinc finger nucleases (ZFNs) are synthetic enzymes comprising three (or more) zinc finger domains linked together to create an artificial DNA-binding protein that binds>9 bp of DNA. To cut DNA, the zinc finger domains are fused to one half of the Fokl nuclease domain such that when two ZFNs bind the two unique 9 bp sites, separated by a suitable spacer, they can cut within the spacer to make a DSB. Methods of designing zinc finger nucleases to recognize a desired target are well known in the art and are described in more detail in, e.g, U.S. Pat. No. 7,163,824 to Cox III; U.S. Patent Application Publication No. 2017/0327795 to Kim et al.; and Harrison et al., “A Beginner's Guide to Gene Editing,” Exp. Physiol. 103(4):439-448 (2018), each of which is hereby incorporated by reference in its entirety.

The recombinant genetic constructs described herein further comprise first and second “gene sequences” also referred to herein as “homology arms”. These gene sequences, direct insertion of the recombinant construct into a gene of interest (i.e., a target gene) within the MSCs by, for example, homologous recombination. Thus, the recombinant genetic construct comprises a first gene sequence that is located 5′ to the promoter region of the native/endogenous genes encoding one of the immunomodulatory proteins; and the second gene sequence is located immediately upstream (or 5′) to exon 1 of the native/endogenous gene encoding one of the immunomodulatory proteins.

The first and second gene sequence(s) of the recombinant genetic construct described herein are nucleotide sequences that are the same as or closely homologous (sharing significant sequence identity) to the nucleotide sequence of particular regions of the target gene, i.e., the gene into which the recombinant genetic construct will be inserted.

Preferably, the first and second gene sequences of the recombinant construct are the same as or similar to the target gene sequence (e.g., the same as the sense strand of the target gene) immediately upstream and downstream of an insertion cleavage site.

In some embodiments, the percent identity between the first gene sequence located at the 5′ end of the recombinant construct (i.e., a 5′ homology arm) and the corresponding sequence of target gene (e.g., sense strand) is at least about 70%, 75%, 80%, 85%, 90%, 95%, 98%, 99%, or 100%. In some embodiments, the percent identity between the second gene sequence located at the 3′ end of the recombinant construct (i.e., a 3′ homology arm) and the corresponding sequence of the target gene (e.g, sense strand) is at least about 70%, 75%, 80%, 85%, 90%, 95%, 98%, 99%, or 100%.

In some embodiments, the first and second gene sequences (e.g, the 5′ and 3′ homology arms) are more than about 30 nucleotide residues in length, for example more than about any of 50 nucleotide residues, 100 nucleotide residues, 200 nucleotide residues, 300 nucleotide residues, 500 nucleotide residues, or 1000 or more nucleotides in length.

The recombinant genetic construct as disclosed herein may be circular or linear.

When the recombinant genetic construct is linear, the first and second gene sequences (e.g, the 5′ and 3′ homology arms) are proximal to the 5′ and 3′ ends of the linear nucleic acid, respectively, i.e., about 200 bp away from the 5′ and 3′ ends of the linear nucleic acid. In some embodiments, the first gene sequence (e.g, the 5′ homology arm) is about any of 1, 2, 5, 10, 15, 20, 25, 30, 35, 40, 45, 50, 55, 60, 70, 80, 90, 100, 120, 140, 160, 180, or 200 nucleotide residues away from the 5′ end of the linear DNA. In some embodiments, the second gene sequence (e.g, the 3′ homology arm) is about any of 1, 2, 5, 10, 15, 20, 25, 30, 35, 40, 45, 50, 55, 60, 70, 80, 90, 100, 120, 140, 160, 180, or 200 nucleotide residues away from the 3′ end of the linear DNA.

The first and second gene sequences of the recombinant genetic construct are designed to mimic sequences of a “target gene” to facilitate insertion of the construct into the target gene. As described above, in particular, for replacement of the native promoter sequence of a native/endogenous gene for an immunomodulatory protein with a constitutive promoter sequence, thereby increasing expression of the native immunomodulatory protein, the targeted sequences are located in the upstream or 5′ region of the native/endogenous gene.

The eMSCs that are successfully prepared using the above techniques can be selected for using the selection markers or otherwise isolated using cell sorting procedures that are well known in the art. Prior to use, the eMSCs can optionally be cultured to induce formation of eMSC spheroids. For example, the eMSCs can be cultured using methylcellulose-based medium or hydrogels such as alginate, fibrin, collagen, or hyaluronic acid to promote aggregation. See Ryu et al., “Spheroid Culture System Methods and Applications for Mesenchymal Stem Cells,” Cells 8(12):1620 (2019); Deynoux et al., “A Comparative Study of the Capacity of Mesenchymal Stromal Cell Lines to Form Spheroids,” PLoS One 15(6):e0225485 (2020), each of which is hereby incorporated by reference in its entirety. Alternatively, a number of handling technique can alternatively be utilized, as reported by Ryu et al., “Spheroid Culture System Methods and Applications for Mesenchymal Stem Cells,” Cells 8(12):1620 (2019), which is hereby incorporated by reference in its entirety.

Once the eMSCs are selected, they can then be used to prepare a mixed cell population that includes the eMSCs (whether in the form of spheroids or not) and one or more cell types distinct of the eMSCs.

The one or more cell types that are distinct of the eMSCs can be harvested and cultured non-recombinant cells or they may also be recombinantly modified.

In certain embodiments, the one or more cell types that are present in the mixed cell population includes islet cells. The islet cells may be any one or more of a cells, β cells, δ cells, PP cells, and ε cells. The islet cells can also be a combination of the various islet cells in the form of one or more islets.

According to one embodiment, the mixed cell population includes either one or both of a cells and p cells, and non-spheroidal eMSCs of the invention.

According to another embodiment, the mixed cell population includes either one or both of a cells and p cells, and spheroidal eMSCs of the invention.

In certain embodiments, the mixed cell population can be cultured in vitro.

In alternative embodiments, as discussed infra, the mixed cell population can be introduced into the body of a patient (i.e., reside in vivo).

In the preparation of eMSCs and mixed cell populations, the cells are preferably mammalian in origin, e.g., a preparation of rodent cells (i.e., mouse or rat cells), rabbit cells, guinea pig cells, feline cells, canine cells, porcine cells, equine cells, bovine cells, ovine cells, non-human primate cells, or human cells. In one embodiment, the preparation is a preparation of human cells.

While the immunomodulatory capacity of the eMSCs to control cellular or acellular mediated destruction of the one or more cell types that are distinct of the eMSCs, further protection may be afforded by introducing the mixed cell population into an implantable cell culture device that includes a culture chamber and presents a physical barrier against T cell infiltration while allowing expression products of the eMSCs and the one or more cell types that are distinct of the eMSCs to diffuse through the physical barrier. For example, such an implantable device may include one or more porous coatings that permit passage of soluble factors but inhibit passage of cells. By way of example, islet cells can be protected against T-cell mediated destruction, while insulin and/or glucagon can diffuse through the porous coatings in response to signals.

Exemplary implants of this type include, without limitation, those disclosed in PCT Publ. Nos. WO/2016/019391, WO/2015/191547, and WO/2021/202945; and U.S. Publ. Nos. 20170095514 and 20200171095, each of which is hereby incorporated by reference in its entirety. Other implants having different constructions and containing different materials can also be utilized.

As used herein, a “subject” or “individual” or a “patient” that receives a preparation of eMSCs described herein (whether as part of a mixed cell population or not) encompasses any animal, preferably a mammal. Suitable subjects include, without limitation, domesticated and undomesticated animals such as rodents (mouse or rat), cats, dogs, rabbits, horses, cows, sheep, pigs, and any primates. In one embodiment the subject is a human subject. Suitable human subjects include, without limitation, infants, children, adults, and elderly subjects.

In one embodiment, the subject is in need of a terminally differentiated cell type. For example, the subject has a condition mediated by the loss of or dysfunction of a differentiated cell population. One exemplary condition that includes the partial or complete loss of a differentiated cell type is Type 1 diabetes, where β cells are lost.

In carrying out the methods of the present disclosure, “treating” or “treatment” includes inhibiting, preventing, ameliorating or delaying onset of a particular condition. Treating and treatment also encompasses any improvement in one or more symptoms of the condition or disorder. Treating and treatment encompasses any modification to the condition or course of disease progression as compared to the condition or disease in the absence of therapeutic intervention.

In some embodiments, the administering is effective to reduce at least one symptom of a disease or condition that is associated with the loss or dysfunction of the differentiated cell type. In another embodiment, the administering is effective to mediate an improvement in the disease or condition that is associated with the loss or dysfunction of the differentiated cell type. In another embodiment, the administering is effective to prolong survival in the subject as compared to expected survival if no administering were carried out.

In accordance with this aspect of the present disclosure, the preparation of eMSCs may be autologous/autogeneic (“self”) to the recipient subject. In another embodiment, the preparation of eMSCs is non-autologous (“non-self,” e.g., allogeneic, syngeneic, or xenogeneic) to the recipient subject. The one or more cell types that are distinct of the eMSCs and are present in the mixed cell population are, in most cases, non-autologous.

In carrying out the methods of the present disclosure, the administering may be carried out in the absence of immunosuppression or using a modified course of immunosuppression therapy such as a reduction in the frequency, quantity, or number of immunosuppressive agents administered to the individual. For example, in one embodiment, the administering may be followed up with an initial course of immunosuppression therapy, but the administration of long-term immunosuppression therapy is not required.

The number of cells in a given volume can be determined by well-known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The preparation eMHCs of the mixed population of cells can be administered in dosages and by techniques well known to those skilled in the medical and veterinary arts taking into consideration such factors as the age, sex, weight, and condition of the particular patient, and the formulation that will be administered. The dose appropriate to be used in accordance with various embodiments described herein will depend on numerous factors. It may vary considerably for different circumstances. The parameters that will determine optimal doses to be administered for primary and adjunctive therapy generally will include some or all of the following: the disease being treated and its stage; the species of the subject, their health, gender, age, weight; the subject's immunocompetence; other therapies being administered; and expected potential complications from the subject's history or genotype. The parameters may also include whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as immunosuppressants). The optimal dose in a given situation also will take into consideration the way in which the cells/medium are formulated, the way they are administered, and the degree to which the cells/medium will be localized at the target sites following administration. Finally, the determination of optimal dosing necessarily will provide an effective dose that is neither below the threshold of maximal beneficial effect nor above the threshold where any deleterious effects associated with the dose outweighs the advantages of the increased dose.

For fairly pure preparations of eMHCs, optimal doses in various embodiments will range from about 10⁴ to about 10⁹ cells/spheroids per administration. In some embodiments, the optimal dose per administration will be between about 10⁵ to about 10⁷ cells/spheroids. Where the mixed cell populations are administered, these same doses may apply but the mixture will include between about 2 to about 40 percent of the eMSCs/spheroids, preferably between about 5 to about 30 percent of the eMSCs/spheroids, such as about 5 to about 25 percent, about 5 to about 20 percent, about 5 to about 15 percent, about 7.5 to about 30 percent, about 7.5 to about 25 percent, about 7.5 to about 20 percent, about 7.5 to about 15 percent, about 10 to about 30 percent, about 10 to about 25 percent, about 10 to about 20 percent, about 12.5 to about 30 percent, about 12.5 to about 25 percent, or about 12.5 to about 20 percent.

It is to be appreciated that a single dose of the eMSCs or mixed cell population may be delivered all at once, fractionally, or continuously over a period of time. The entire dose also may be delivered to a single location or spread fractionally over several locations.

Human and non-human subjects are treated generally longer than experimental animals; but treatment generally has a length proportional to the length of the disease process and the effectiveness of the treatment. Those skilled in the art will take this into account in using the results of other procedures carried out in humans and/or in animals, such as rats, mice, dogs, cats, cows, horses, non-human primates, and the like, to determine appropriate doses for such subjects. Such determinations, based on these considerations and taking into account guidance provided by the present disclosure and the prior art will enable the skilled artisan to do so without undue experimentation and for purposes of optimizing a treatment regimen.

Suitable regimens for initial administration and further doses or for sequential administrations may all be the same or may be variable. Appropriate regimens can be ascertained by the skilled artisan, from this disclosure, the documents cited herein, and the knowledge in the art.

In some embodiments, the preparation of eMSCs or mixed cell population is administered to a subject in one dose. In others, the preparation of eMSCs or mixed cell population is administered to a subject in a series of two or more doses in succession. In some other embodiments where the preparation of cells is administered in a single dose, in two doses, and/or more than two doses, the doses may be the same or different, and they are administered with equal or with unequal intervals between them.

The preparation of eMSCs or mixed cell population may be administered in many frequencies over a wide range of times. In some embodiments, they are administered over a period of less than one day. In other embodiments, they are administered over two, three, four, five, or six days. In some embodiments, they are administered one or more times per week, over a period of weeks. In other embodiments, they are administered over a period of weeks for one to several months. In various embodiments, they may be administered over a period of months. In others they may be administered over a period of one or more years. Generally, lengths of treatment will be proportional to the length of the disease process, the effectiveness of the therapies being applied, and the condition and response of the subject being treated.

The choice of formulation for administering the eMSCs or mixed cell population for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Thus, measures may be taken to increase cell survival and/or to overcome problems posed by barriers to seeding and/or growth.

Final formulations may include an aqueous suspension of cells/medium and, optionally, protein and/or small molecules, and will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose, and the like. Organic polymer base materials, such as polyethylene glycol and hyaluronic acid as well as non-fibrillar collagen, such as succinylated collagen, can also act as lubricants. Such lubricants are generally used to improve the injectability, intrudability, and dispersion of the injected material at the site of injection. This final formulation is by definition the eMSCs or mixed cell population described herein in a pharmaceutically acceptable carrier.

Based on the foregoing, the eMSCs can be used in accordance with the present invention to improve the survival of transplanted cells. This method includes the step of implanting (i) one or more eMSCs, and (ii) one or more cell types distinct of the eMSCs into an individual at the same locus, whereby the implanted one or more cell types distinct of the eMSCs exhibit improved survival compared to the same one or more cell types implanted in the absence of the one or more eMSCs at the same locus.

According to one embodiment, the eMSCs or the mixed population of cells can be administered via implantation at a particular locus (e.g., at the renal capsule). Alternatively, the eMSCs or the mixed population of cells can be present in a cell culture device of the type described above, which cell culture device is implanted within the renal capsule or adjacent to the kidneys. Where implantation is used, the implantation can involve an open surgical field or a laparoscopic procedure. Alternatively, the eMSCs or the mixed population of cells can be administered intraperitoneally, percutaneously, or subcutaneously.

A further aspect relates to a method of treating a diabetic subject, such as an individual having Type 1 diabetes. This method includes the step of implanting the eMSCs or the mixed population of cells (containing one or more islet cells or islets) into the diabetic subject, whereby the islet cells express insulin, glucagon, or both to treat the diabetic subject. Administration by implantation can be carried out using the procedures noted above, with or without the presence of a cell culture device.

Yet another aspect relates to a method of modifying T cell response to an allograft. This method includes the step of implanting an allograft into an individual with one or more eMSCs, whereby the one or more eMSCs cause, relative to an allograft in the absence of the one or more eMSCs, (i) an increase in the percentage of regulatory T cells (CD4+/CD25+/Foxp3+) present in the implanted graft, and (ii) a reduction in the number of T effector cells (CD4+ or CD8+) present in the implanted graft. In certain embodiments, the one or more eMSCs also cause, relative to an allograft in the absence of the one or more eMSCs, a reduction in the number of activated dendritic cells (CD11c+/CD86+) present in the implanted graft.

The implanted allograft can take the form of the mixed cell population as described above, or the eMSCs located in proximity to distinct cells of the allograft. In preferred embodiments, the allograft comprises islet cells, islets, or a mixture of the islet cells or islets with the one or more eMSCs/spheroids.

Wherever the word ‘about’ is employed herein, for example in the context of numbers or amounts, i.e., absolute amounts such as sizes, nucleotide length, doses, or concentrations, or time periods; or relative amounts including percentages, it will be appreciated that such variables are approximate and as such may vary by ±10%, for example ±5% and preferably ±2% (e.g. ±1%) from the actual numbers specified. In this respect, the term ‘about 10%’ means e.g. ±10% about the number 10, i.e. between 9% and 11%.

### EXAMPLES

The examples below are intended to exemplify the practice of embodiments of the disclosure but are by no means intended to limit the scope thereof.

### Materials and Methods for Examples 1-5

Experimental Design: Examples 1-5 demonstrate the development of a type of immunoprotective accessory cell that can protect allogeneic islets with no or reduced systemic immunosuppression. Animals were handled and cared for by trained scientists and approved by the Cornell Institutional Animal Care and Use Committee. Sample size, including number of mice per group, was chosen to ensure adequate power and was based on historical data. All mice used were males to eliminate any potential confounding influences of gender differences. All mice were randomly assigned to treatment groups, and all data collection and analyses were performed blindly for different treatment conditions. The number of biologic replicates is specified in the figure legends.

Animals: Eight-week-old male C57BL/6, BALB/c, and FVB-Tg(CAG-luc,-GFP)L2G85Chco/J (L2G85) mice were purchased from the Jackson Laboratory (Bar Harbor, ME). All animal procedures were approved by the Cornell Institutional Animal Care and Use Committee.

Cell Culture: Strain C57BL/6 mouse MSCs (Cyagen, MUBMX-01001) were purchased. The 293T cell line and the 4T1 cell line were received as gifts. 293T cells were cultured in Dulbecco's modified Eagle's medium (Gibco, 2051526) supplemented with 10% FBS and 1% penicillin/streptomycin (P/S). 4T1 cells were cultured in RPMI 1640 media with 10% FBS and 1% P/S. MSCs were cultured in MSC growth medium (Cyagen, GUXMX-90011) following the manufacturer's instruction. Primary islets were cultured in RPMI 1640 media with 10% FBS and 1% P/S. Splenocytes were cultured in RPMI 1640 media (Thermo Fisher Scientific, 11875093) with 5% FBS, 1% P/S, 1% L-glutamine (Thermo Fisher Scientific, 25030149), and 0.1% 2-mercaptoethanol (Thermo Fisher Scientific, 31350010).

Generation of GFP Luciferase-expressing Cell Line: Plasmid containing enhanced GFP gene (720 base pairs (bp), e.g., Genbank Accession AAK15492, which is hereby incorporated by reference in its entirety) and humanized firefly luciferase (Luc2) gene (1653 bp, e.g., Genbank Accession AHL68682, which is hereby incorporated by reference in its entirety) was constructed by Vector Builder. GFP⁺/luciferase⁺4T1 cells and GFP⁺/luciferase⁺ MSCs were generated following a previous publication (Wang et al., “A Nanofibrous Encapsulation Device for Safe Delivery of Insulin-producing Cells to Treat Type 1 Diabetes,” Sci. Transl. Med. 13:eabb4601 (2021), which is hereby incorporated by reference in its entirety) and verified under a fluorescence microscope.

Generation of PD-L1 and CTLA4-Ig-expressing Cell Lines: The pLenti-based expression vector containing mouse PD-L1 gene (873 bp, e.g., Genbank Accession GQ904196, which is hereby incorporated by reference in its entirety) and CTLA4-Ig gene (1179 bp) was constructed by request using the services of Vector Builder. 293T cells were plated and cultured in 10-cm treated tissue culture plate the day before transduction to obtain 90 to 95% confluency. The lentiviral stocks were produced by transfecting 293T cells with the designed vector using the ViraPower Bsd Lentiviral Support Kit (Thermo Fisher Scientific, K497000) following the manufacturer's instruction. After 48 to 72 hours after transfection, the lentiviral supernatant was collected, centrifuged at 3000 rpm for 15 min at 4° C. to pellet debris, and stored at −80° C. MSC single cells were prepared and seeded in a six-well plate (5000 cells per well) with 2 ml of virus-containing supernatant. After 48 hours of transduction, lentivirus medium was discarded, and fresh MSC culture medium was added. Bsd solution with a final concentration of 5 μg/ml was used to purify the transfected cells. The PD-L1/CTLA4-Ig GFP/luciferase 4T1 cells were generated in the same manner as PD-L1/CTLA4-Ig MSCs. For the formation of MSC spheroids, about 4 ml of solution containing 4 million MSCs was added in one well of six-well suspension culture plate (Genesee Scientific, 25-100). Then, the cells were cultured on an orbital shaker with a speed of 100 rpm overnight. The spheroids were collected and centrifuged into cell pellet for further use.

Quantitative Reverse Transcription PCR: Five million MSCs or eMSCs were collected in the tube and centrifuged into a cell pellet. Total RNA of two groups was isolated with an RNeasy kit (Qiagen, 74106), and complementary DNA was prepared using reverse transcriptase III (Thermo Fisher Scientific, 4368814) according to the manufacturer's instruction. Quantitative PCR was performed using SYBR Green Master Mix (Thermo Fisher Scientific, A25776), and detection was achieved using the StepOnePlus Real-time PCR system thermocycler (Applied Biosystems). Expression of target genes was normalized to glyceraldehyde-3-phosphate dehydrogenase (GAPDH). Real-time PCR primer sequences are listed in Table 1 below.

TABLE 1
Sequences of primers used for qRT-PCR

| Gene | Forward (5′ to 3′) | Reverse (5′ to 3′) |
|---|---|---|
| GAPDH | AGGTCGGTGTGAACGGATTTG | GGGGTCGTTGATGGCAACA |
| | (SEQ ID NO: 21) | (SEQ ID NO: 22) |
| CTLA4 | AGCAGTAGATACGGGGCTCT | TATCGGGACACGGTTCAGGA |
| | (SEQ ID NO: 23) | (SEQ ID NO: 24) |
| PD-L1 | GCTCCAAAGGACTTGTACGTG | TGATCTGAAGGGCAGCATTTC |
| | (SEQ ID NO: 25) | (SEQ ID NO: 26) |
| TGF-β1 | GGCCAGATCCTGTCCAAGC | GTGGGTTTCCACCATTAGCAC |
| | (SEQ ID NO: 27) | (SEQ ID NO: 28) |
| Arg-1 | CATTGGCTTGCGAGACGTAGAC | GCTGAAGGTCTCTTCCATCACC |
| | (SEQ ID NO: 29) | (SEQ ID NO: 30) |
| iNOS | GAGACAGGGAAGTCTGAAGCAC | CCAGCAGTAGTTGCTCCTCTTC |
| | (SEQ ID NO: 31) | (SEQ ID NO: 32) |
| TNF-α | GGTGCCTATGTCTCAGCCTCTT | GCCATAGAACTGATGAGAGGGAG |
| | (SEQ ID NO: 33) | (SEQ ID NO: 34) |

Western Blot: Five million MSCs or eMSCs were collected in the tube and centrifuged into a cell pellet. Cells were lysed with radioimmunoprecipitation assay lysis buffer (Thermo Fisher Scientific, 89901) in the presence of protease inhibitor. The concentration of extracted protein was measured using Pierce 660-nm protein assay reagent (Thermo Fisher Scientific, 1861426) and the Bio-Rad SmartSpec 3000 spectrophotometer. Forty micrograms of whole-cell protein lysate was applied to 4 to 15% Mini-PROTEAN TGX precast protein gel (Bio-Rad, 4561084) with electrophoresis and then transferred to a nitrocellulose membrane. The probed primary antibodies were detected by using horseradish peroxidase-conjugated secondary antibodies and the enhanced chemiluminescent detection system (GE Healthcare, 28906836). Primary and secondary antibodies are described in Table 2 below.

TABLE 2
List of antibodies used in the western blot

| Antibody | Vendor | Catalog | Dilution | |
|---|---|---|---|---|
| Goat anti-mouse | R&D Systems | AF476-SP | 0.1 | μg/mL |
| CTLA-4 antibody | | | | |
| Rabbit anti-mouse | R&D Systems | MAB90781-SP | 2 | μg/mL |
| PD-L1 antibody | | | | |

| | | | |
|---|---|---|---|
| Rabbit anti-α | Cell Signaling | 2144S | 1:1000 |
| tubulin antibody | | | |
| Horse anti-rabbit | Cell Signaling | 7074S | 1:1000 |
| IgG HRP | | | |
| Horse anti-goat | Vector Laboratories | PI-9500-1 | 1:1000 |
| IgG HRP | | | |

Enzyme-linked Immunosorbent Assay: One million MSCs or eMSCs were seeded in one well of a 12-well culture plate with 1 ml of culture medium in each well. Cells were cultured for 6 hours in an incubator. Culture media were collected in tubes, and the supernatant was collected after centrifuge. The CTLA4-Ig in the supernatant was quantified by Mouse CTLA-4 DuoSet ELISA (R&D Systems, DY476) according to the manufacturer's instruction. Absorbance of reaction solution at 450 nm was measured in the Synergy plate reader (Biotek).

Flow Cytometry: For analysis of MSCs and 4T1 cells before and after modification, the cells were detached from the culture dish by using TrypLE, centrifuged, and washed with PBS. The cells were blocked with staining buffer, incubated for 15 min at 4° C. with antibodies shown in Table 3 below, washed with staining buffer, and resuspended in staining buffer to analyze on an Attune N×T flow cytometer (Thermo Fisher Scientific). Native 4T1 cells engrafted in BALB/c mice and modified 4T1 cells engrafted in C57BL/6 mice were dissociated into single cells with mechanical force. The cells were filtered through a 40-μm strainer (VWR, 10199-654). The single cells were then stained with antibody (APC anti-mouse PD-L1 antibody, BioLegend, 124311) and analyzed following the steps as described above. The data were analyzed and generated by FlowJo™ software v10.7.

TABLE 3
List of antibodies used in the flow cytometry

| Antibody | Vendor | Catalog |
|---|---|---|
| FITC anti-mouse IgG Antibody | Biolegend | 406001 |
| FITC anti-mouse CD29 antibody | Biolegend | 102205 |
| FITC anti-mouse Ly-6A/E (Sca-1) Antibody | Biolegend | 108105 |
| FITC anti-mouse/human CD44 antibody | Biolegend | 103021 |
| FITC anti-mouse CD31 Antibody | Biolegend | 102405 |
| FITC anti-mouse CD45 Antibody | Biolegend | 103107 |
| FITC anti-mouse CD117 antibody | Biolegend | 105805 |
| APC anti-mouse PD-L1 antibody | Biolegend | 124311 |

Immunofluorescent Staining: Cells were seeded in eight-well Lab-Tek chamber slides at a density of 50,000 cells/cm². After confluency, cells were fixed in 10% formalin and then blocked for 30 min in 5% donkey serum (Sigma-Aldrich, S30-M). Subsequently, cells were incubated with primary antibodies (rabbit anti-mouse PD-L1 antibody, R&D Systems, MAB90781-SP) for 30 min at room temperature. Cells were washed three times with PBS and then incubated with secondary antibodies [donkey anti-rabbit immunoglobulin G (H+L) highly cross-adsorbed secondary antibody, Alexa Fluor 488, Thermo Fisher Scientific, A21206] for 30 min at room temperature. Cells were washed three times with PBS and stained with 4′,6-diamidino-2-phenylindole (DAPI) (0.5 μg/ml) for 5 min. Slides were mounted with fluorescence mounting medium (Sigma-Aldrich, F6057). Slides were imaged using confocal microscopy (FV1000, Olympus, Japan).

In vitro T CellSuppression Assay: MSCs or eMSCs were seeded at a density of 20,000 per well in a U-bottom 96-well plate and incubated for 2 hours in MSC culture media. Splenocytes were isolated from either BDC2.5 TCR transgenic mice (CD4 T cells specific for BDC2.5 mimotope on MHCII) or from NY8.3 TCR transgenic mice (CD8 T cells specific for IGRP peptide on MHCI) and labeled using CellTrace™ Violet (cell proliferation kit, Invitrogen, San Diego, CA, USA) for cell proliferation quantification by CellTrace dilution. The MSC media were removed. Splenocytes were added at a density of 100,000 per well into each well on top of the MSCs using culture medium for splenocytes. Splenocytes were stimulated either with BDC2.5 (5 μg/ml) peptide (sequence: RTRPLWVRME, SEQ ID NO: 35) or with IGRP (0.1 g/ml) peptide (sequence: VYLKTNVFL, SEQ ID NO: 36) in the presence of MSCs or eMSCs for 3 days. After 3 days' coculture, the splenocytes were harvested and stained for flow cytometry analysis using LIVE/DEAD Fixable Dead Cell Stain (near infrared, Invitrogen, L34975) and antibodies against the following surface markers: anti-mouse CD3 (BD Biosciences, 555274), anti-mouse CD4 (eBioscience, 56004182), anti-mouse CD8 (BD Biosciences, 551182), anti-mouse CD44 (BD Biosciences, 582464), anti-mouse CD25 (BD Biosciences, 552880), and granzyme B (BioLegend, 515406). Live, CD3⁺ CD4⁻ CD8⁺, and CD3⁺ CD4⁺ CD8⁻ T cell subpopulations were identified, and their proliferation in vitro was quantified by CellTrace™ dilution. Activation was determined by CD25 and CD44 up-regulation for CD4 and by CD25, CD44, and granzyme B up-regulation for CD8. Flow cytometry was performed with a CytoFlex™ S flow cytometer (Beckman Coulter, Brea, CA, USA), and data were analyzed with FlowJo™ (Tree Star Inc., Ashland, Oregon, USA).

Live and Dead Staining: Fifty islets without MSCs, with MSC spheroids, or with eMSC spheroids were cultured in 3 ml of RPMI 1640 complete media for 24 hours in nonadherent 25-mm² culture dishes. After culture, islets were handpicked and stained by calcein-AM (green, live) and ethidium homodimer (red, dead) according to the manufacturer's protocol (R37601, Thermo Fisher Scientific). Fluorescent microscopic images were taken using a digital inverted microscope (EVOS FL Cell Imaging System). Quantification of the percentage of live cells in islets was carried out by calculating the intensity of fluorescence using ImageJ.

In vitro Glucose-Stimulated Insulin Secretion: Fifty islets without MSCs, with MSC spheroids, or with eMSC spheroids were cultured in 3 ml of RPMI 1640 complete media for 24 hours in nonadherent 25-mm² culture dishes. After culture, islets were handpicked and incubated in prewarmed Krebs-Ringer bicarbonate solution supplemented with 25 mM Hepes, 1 mM L-GlutaMAX, 0.1% BSA, and 2.8 mM D-glucose for 30 min at 37° C., 5% CO² for calibration, and then incubated for 1 hour with 2.8 mM or 16.7 mM D-glucose under the same condition. The supernatant was collected and frozen for future analysis. The insulin content in the supernatant was quantified by mouse insulin ELISA kit (ALPCO) according to the manufacturer's specification. Absorbance of reaction solution at 450 nm was measured in the Synergy plate reader (Biotek). The SI was calculated as the ratio of insulin secretion at high glucose (16.7 mM) to that at low glucose (2.8 mM).

Bioluminescent Imaging: At different time points after transplantation, the mice were injected with luciferin (150 mg/kg body weight; PerkinElmer, 122799) and imaged with the IVIS Spectrum System (PerkinElmer) at the Biotechnology Resource Center at Cornell.

Isolation of Rodent Pancreatic Islets: Mouse pancreatic islets were isolated from 8-week-old male BALB/c mice or L2G85 mice. One bottle of collagenase (Vitacyte, CIzyme RI, 005-1030) was reconstituted in 30 ml of M199 media (Gibco, USA). The bile duct was cannulated with a 27-gauge needle, and the pancreas was distended with cold collagenase. The perfused pancreases were then removed and digested in a 37° C. water bath for 21 min. Islets were centrifuged in lymphocyte separation medium (Corning, 25-072-CV)/M199 media gradient in 1750 rcf for 20 min at 4° C. Purified islets were hand-counted by aliquot under a stereomicroscope (Olympus SZ61). Detailed procedures of isolation and purification were described in previous publications (Wang et al., “A Nanofibrous Encapsulation Device for Safe Delivery of Insulin-producing Cells to Treat Type 1 Diabetes,” Sci. Transl. Med. 13:eabb4601 (2021); Wang et al., “Scaffold-supported Transplantation of Islets in the Epididymal Fat Pad of Diabetic Mice,” J. Vis. Exp. 54995 (2017), each of which is hereby incorporated by reference in its entirety).

Chemically Induced Diabetic Mouse Model: To create diabetic mice, mice were injected intraperitoneally with freshly prepared STZ (Sigma-Aldrich, 130 mg/kg body weight) solution (13 mg/ml in 5 mM sodium citrate buffer solution). A small drop of blood was collected from the tail vein using a lancet and tested using a commercial glucometer (Contour next, Ascensia Diabetes Care, NJ). Only mice whose nonfasted blood glucose concentrations were above 300 mg/dl with two consecutive measurements were considered diabetic and underwent transplantation.

Injection of 4T1 Cell Line: The 4T1 cells were detached from the culture dish using TrypLE. Half-million native 4T1 cells or modified 4T1 cells were suspended in 50 μl of 50% Matrigel (Corning, 354277). Then, cell suspension was transferred to a 0.5-ml syringe and injected into the muscle of the right hindlimb.

Preparation of Islet Samples for Transplant: Under an inverted microscope, islets were hand-picked and transferred into each microcentrifuge tube (150 to 200 IEQ/tube for syngeneic islet transplantation; 500 to 600 IEQ/tube for allogeneic islet transplantation). MSC or eMSC spheroids (500 to 600) were added into each tube. The samples were centrifuged and washed with PBS to remove the culture medium. The islet and cell pellets in each tube were resuspended in 50 μl of PBS. Then, the islet and cell suspensions in each tube were loaded into polyethylene (PE) tubing (BD Biosciences, 63018-668). The PE tubing was centrifuged at 1000 rpm for 10 min and placed on ice.

Cell Transplantation in Kidney Capsule: The diabetic mice were anesthetized with isoflurane, and the left flank of the mouse was shaved. The surgical area was sanitized with povidone iodine swab and ethanol swab. The left kidney was located, and a small incision was made in the skin and then in the peritoneum to expose the left kidney. The kidney was popped out of the peritoneum when a slight pressure was applied to the incision. The PE tubing was carefully slid under the kidney capsule making a small pocket to hold the islet samples. The islets with or without cell samples were slowly loaded into the kidney capsule. The kidney was gently put back into the peritoneum before closing the incision with suture and skin staples. After surgery, mice were monitored twice a week, and blood glucose was detected. A total of 500 to 600 GFP/luciferase MSC spheroids or the corresponding number of MSC single cells were transplanted in the kidney capsule of healthy C57BL/6 mice using the described methods.

Intraperitoneal Glucose Tolerance Test: Mice were fasted overnight before receiving an intraperitoneal glucose bolus (2 g/kg body weight). The healthy mice and diabetic mice were used as positive control and negative control, respectively. Blood glucose was monitored at regular intervals (time: 0, 15, 30, 60, 90, and 120 min) after injection, allowing for the area under the curve to be calculated and analyzed between groups.

Immune Profiling: The following procedures were previously described (Wang et al., “A Nanofibrous Encapsulation Device for Safe Delivery of Insulin-producing Cells to Treat Type 1 Diabetes,” Sci. Transl. Med. 13:eabb4601 (2021), which is hereby incorporated by reference in its entirety). BALB/c islets (500 IEQ) without MSCs or with MSC spheroids or with eMSC spheroids were transplanted in diabetic C57BL/6 mice in the kidney capsule. Eight days after transplantation, the kidney transplanted with islets was retrieved. The tissue containing islets was sliced from the kidney using scissors and digested with type I collagenase (1 mg/ml) (Worthington Biochemical Corporation) for 1 hour in an incubator. The digestion was stopped by adding cell culture medium containing 10% FBS. The digested tissue was smashed, and the cell solution was filtered through a Falcon 40-μm strainer (Corning, 431750) to obtain single cells. Cells were centrifuged at 1000 rpm for 5 min. Supernatant was discarded, and cells were washed with PBS solution to remove the remaining FBS. The cells were stained with the Zombie™ Yellow Fixable Viability Kit (BioLegend, 423103) following the manufacturer's instruction. Cells were washed with 2 ml of cell-staining buffer (BioLegend, 420201) and centrifuged into a pellet. Fc receptors were blocked by preincubating cells with TruStain™ FcX PLUS (anti-mouse CD16/32) antibody (BioLegend, 156603) in 100 μl of cell-staining buffer for 5 min on ice. Then, cells were labeled with mixed antibodies (see Table 4 below) on ice for 30 min. The cells were washed twice with 2 ml of cell-staining buffer by centrifugation at 350 rcf for 5 min. The cells were further stained with Foxp3 using the Foxp3/transcription factor staining buffer set (Thermo Fisher Scientific, 00-5523-00) according to the manufacturer's instruction. Samples stained with fluorescence minus one for intracellular staining were run with every collection. UltraComp™ eBeads Compensation Beads (Thermo Fisher Scientific, 01-2222-41) incubated with each antibody following the manufacturer's instruction were used for compensation. Last, stained cells were analyzed using an Attune™ N×T flow cytometer (Thermo Fisher Scientific). The data were analyzed by FlowJo™ software v10.7.

TABLE 4
List of antibodies used in the immune profiling

| Antibody | Color | Dilution | Company | Part # |
|---|---|---|---|---|
| anti-mouse CD62L | FITC | 1:100 | BioLegend | 104405 |
| anti-mouse CD45 | Brilliant Violet 650 | 1:100 | BioLegend | 103151 |
| anti-mouse CD11c | APC | 1:100 | BioLegend | 117309 |
| anti-mouse CD86 | PE/Cyanine7 | 1:100 | Tonbo Biosciences | 60-0862-U025 |
| anti-mouse CD25 | Brilliant Violet 711 | 1:100 | BioLegend | 102049 |
| anti-mouse CD3 | Pacific Blue | 1:100 | BioLegend | 100213 |
| anti-mouse PD-1 | PE | 1:100 | BioLegend | 135205 |
| anti-mouse CD4 | Percp/Cyanine5.5 | 1:100 | BioLegend | 100433 |
| anti-mouse CD8a | AF700 | 1:100 | BioLegend | 100729 |
| anti-mouse CD44 | APC/Cyanine7 | 1:100 | BioLegend | 103027 |
| anti-mouse Foxp3 | eFlour-615 | 1:100 | Thermo fisher | 42-5773-82 |

Histological Analysis: The following procedures were previously described (Wang et al., “A Nanofibrous Encapsulation Device for Safe Delivery of Insulin-producing Cells to Treat Type 1 Diabetes,” Sci. Transl. Med. 13:eabb4601 (2021), which is hereby incorporated by reference in its entirety). The implants and the spleens were harvested from the mice and fixed in 10% formalin, dehydrated with graded ethanol solutions, embedded in paraffin, and sectioned by Cornell Histology Core Facility. The samples were sliced on a microtome at a thickness of 5 m. The sections were stained with H&E and then imaged by a microscope (IN200TC, Amscope). To conduct immunofluorescent staining, the histological slides were deparaffinized followed by antigen retrieval as described before (Wang et al., “A Bilaminated Decellularized Scaffold for Islet Transplantation: Structure, Properties and Functions in Diabetic Mice,” Biomaterials 138:80-90 (2017), which is hereby incorporated by reference in its entirety). Nonspecific binding was blocked via incubation with 5% donkey serum (Sigma-Aldrich, S30-M) for 1 hour at room temperature. Sections were decanted and incubated with primary antibodies overnight at 4° C. The sections were then washed and incubated with the fluorescence-conjugated secondary antibodies for 1 hour at room temperature. Nuclei were labeled with DAPI, and slides were covered with fluorescent mounting medium (Sigma-Aldrich, F6057). Last, the sections were imaged through confocal microscopy (FV1000, Olympus, Japan). The antibodies used here are listed in Table 5 below. The density of CD3⁺, CD4⁺, CD8⁺, Foxp3⁺, and PD-1⁺ cells and the ratio of CD3⁺ to insulin-producing (INS⁺) cells were analyzed using ImageJ software.

TABLE 5
List of antibodies used in the immunofluorescent staining

| Antibody | Vendor | Catalog |
|---|---|---|
| Rat anti-CD3 antibody | Bio-rad | MCA1477T |
| Rabbit anti-mouse CD4 | Abcam | ab183685 |
| Rabbit anti-CD8 alpha antibody | Abcam | ab209775 |
| Rabbit anti-insulin antibody | Abcam | ab63820 |
| Mouse anti-glucagon antibody | Sigma Aldrich | G2654 |
| Rat anti-FOXP3 antibody | Thermo Fisher | 14-5773-80 |
| Mouse anti-PD-L1 antibody | Thermo Fisher | 66248-1-IG |
| Donkey anti-Rabbit IgG (H + L) | Thermo Fisher | A10042 |
| Highly Cross-Adsorbed | | |
| Secondary Antibody, Alexa Fluor 568 | | |
| Donkey anti-Rat IgG (H + L) | Thermo Fisher | A21208 |
| Highly Cross-Adsorbed | | |
| Secondary Antibody, Alexa Fluor 488 | | |
| Donkey anti-mouse IgG (H + L) | Thermo Fisher | A21202 |
| Highly Cross-Adsorbed | | |
| Secondary Antibody, Alexa Fluor 488 | | |

Statistical Analysis: Unless otherwise stated, data were expressed as means SD. For comparisons between two groups, means were compared using two-tailed Student's t tests. Comparisons between multiple groups were performed by analysis of variance (ANOVA), followed by Tukey's post hoc analysis. Survival curves were analyzed using a Mantel-Cox test. Sample size, including number of mice per group, was chosen to ensure adequate power and was based on literature and historical data. Treated diabetic animals that did not reverse diabetes after allogeneic islet transplantation within 10 days (defined as three consecutive blood glucose readings>300 mg/dl) were excluded from analysis as the failure was not attributable entirely to immune rejection but possibly to variations in islet quality and size, cell numbers, surgery, and other factors (Coronel et al., “Immunotherapy via PD-L1-presenting Biomaterials Leads to Long-term Islet Graft Survival,” Sci. Adv. 6:eaba5573 (2020); Headen et al., “Local Immunomodulation with Fas Ligand-engineered Biomaterials Achieves Allogeneic Islet Graft Acceptance,” Nat. Mater. 17:732-739 (2018), each of which is hereby incorporated by reference in its entirety). All statistical analyses were performed using GraphPad™ Prism v.8 software (GraphPad Software Inc.). The level of significance was assessed starting at P<0.05.

### Example 1—Engineering and Characterizations of MSCs Expressing PD-L1 and CTLA4-Ig

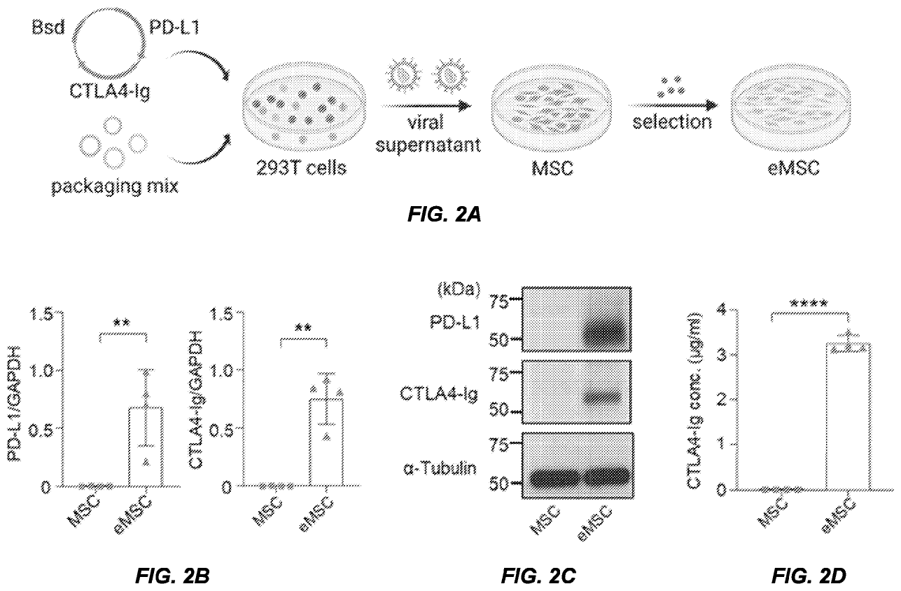

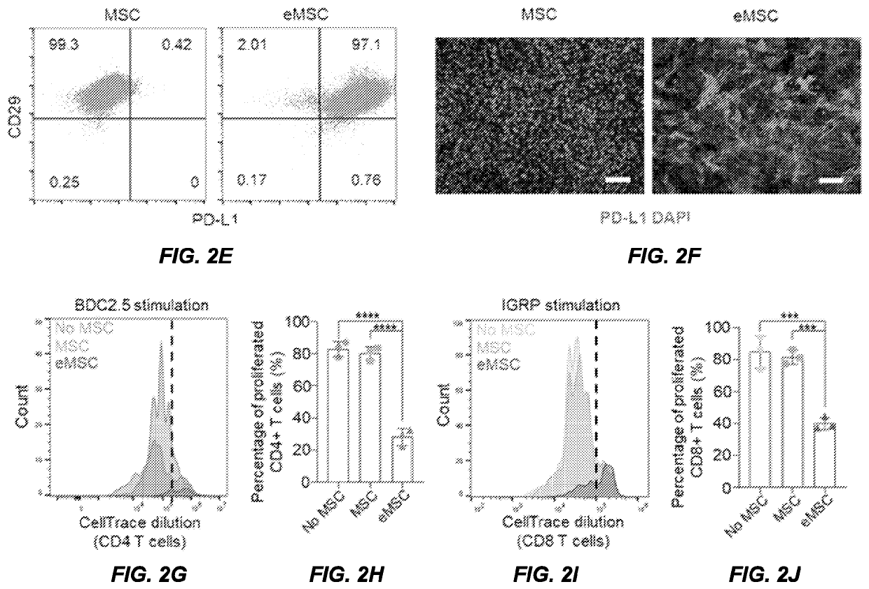

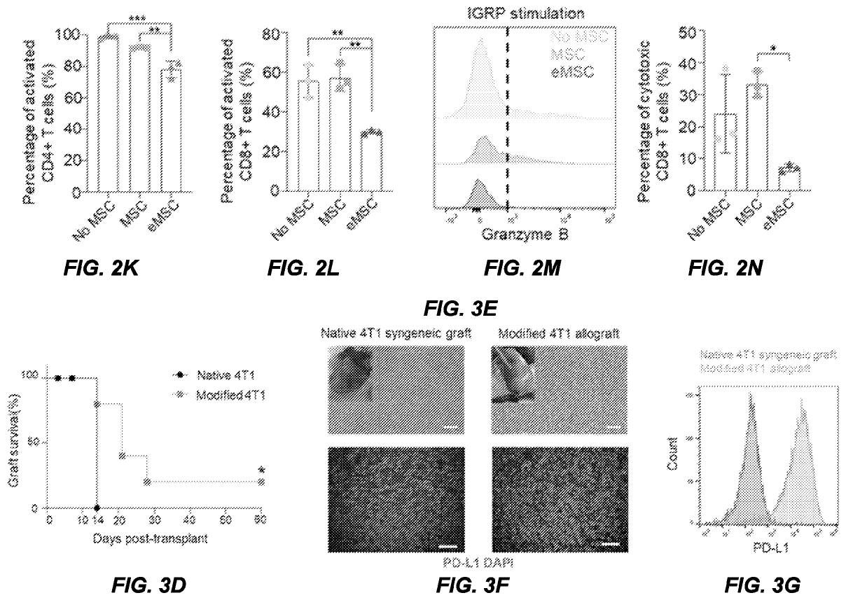

MSCs derived from bone marrow of C57BL/6 mice were chosen as starting cells because MSCs exist abundantly in multiple tissues and have been shown to be promising in multiple therapeutic applications (Bianco et al., “The Meaning, the Sense and the Significance: Translating the Science of Mesenchymal Stem Cells into Medicine,” Nat. Med. 19:35-42 (2013), which is hereby incorporated by reference in its entirety). Before modification, the MSCs were positive for mesenchymal stromal cell markers such as CD29 (99%), SCA-1 (94.4%), and CD44 (99.7%) and negative with CD31 (0.033%), CD45 (0.0065%), and CD117 (0.086%). The eMSCs expressing PD-L1 and CTLA4-Ig were generated by transfection using lentivirus carrying targeted genes (mouse PD-L1 and CTLA4-Ig) and selection through antibiotic blasticidin (Bsd) (FIG. 2A). Gene expression of PD-Li and CTLA4-Ig demonstrated that both genes were incorporated into the genome and highly expressed in the eMSCs, with a 500-fold and 800-fold change compared to MSCs, respectively (FIG. 2B). In the meantime, cell modification did not alter the gene expression of other selected molecules examined such as transforming growth factor β1, arginase 1, inducible nitric oxide synthase, and tumor necrosis factor-α in eMSCs compared to MSCs (P>0.05). Western blot analysis with specific anti-PD-L1 and anti-CTLA4 antibodies verified PD-L1 (55 kDa) and CTLA4 (62 kDa) expressions in the eMSCs (FIG. 2C). Using enzyme-linked immunosorbent assay (ELISA), CTLA4-Ig was detected in the culture medium of eMSCs after in vitro culture for 6 hours, while no CTLA4-Ig was detected in that of MSCs, confirming the secretion of CTLA4-Ig as a soluble factor by the eMSCs (FIG. 2D). Flow cytometry showed that 97.1% eMSCs expressed both CD29 and PD-L1 markers, while almost no expression of PD-L1 was detected on MSCs (FIG. 2E). Immunofluorescent staining images further confirmed the expression of PD-L1 on eMSCs but not MSCs (FIG. 2F).

After verification of PD-L1 and CTLA4-Ig expression by eMSCs, their ability to suppress T cell function was investigated via an in vitro T cell proliferation and activation assay. Allogeneic and diabetogenic splenocytes isolated from either transgenic BDC2.5 nonobese diabetic (NOD) mice [CD4 T cells with transgenic T cell receptor (TCR) specific for the BDC2.5 mimotope presented on MHCII] or from transgenic NY8.3 NOD mice [CD8 T cells with transgenic TCR specific for the islet-specific glucose-6-phosphatase catalytic subunit-related protein (IGRP) peptide presented on MHCI] were cocultured, labeled with a cell proliferation dye (CellTrace™), and pulsed with either the BDC2.5 mimotope or the IGRP peptide, with MSCs or eMSCs at a ratio of 5:1 (splenocytes:MSCs). This was followed by flow cytometry analysis of T cell proliferation and activation, respectively. The data indicated that more than 80% of CD4 T cells and CD8 T cells proliferated when splenocytes were stimulated by the BDC2.5 mimotope and the IGRP peptide, respectively (FIGS. 2G-2J). While MSCs did not have an effect on either CD4 or CD8 T cell proliferation, eMSCs suppressed the proliferation of both CD4⁺ and CD8⁺ allogeneic and diabetogenic T cells compared to MSCs (FIGS. 2G-2J). Quantitative analysis confirmed the proliferation inhibition of both allogeneic and diabetogenic CD4⁺ and CD8⁺ T cells when cocultured with eMSCs (FIGS. 2H, 2J). Similarly, stimulated by the BDC2.5 mimotope or the IGRP peptide, more than 95% CD4 T cells and around 55% CD8 T cells were highly activated as upregulation of CD25 and CD44 (FIGS. 2K, 2L). While MSCs did not have an influence on either CD4 or CD8 T cell activation, eMSCs significantly reduced the percentage of activated CD4 T cells (CD4⁺ CD25⁺ CD44⁺) and activated CD8 T cells (CD8⁺ CD25⁺ CD44⁺) compared to the no-MSC and MSC groups (P<0.01) (FIGS. 2K, 2L). Furthermore, cytotoxic CD8⁺ T cells were identified by their expression of granzyme B. The data showed that around 24 and 33.2% of CD8 T cells were granzyme B⁺ after IGRP stimulation in the no-MSC and MSC groups, respectively (FIGS. 2M, 2N). In contrast, around 7.1% CD8 T cells showed a cytotoxic phenotype as granzyme B expression in the eMSC group. Thus, eMSCs significantly inhibited cytotoxic CD8 T cell generation (CD8⁺ granzyme B⁺) compared to MSCs (P<0.05) (FIGS. 2M, 2N).

### Example 2—PD-L1 and CTLA4-Ig Expression Delays Allogeneic Cell Rejection

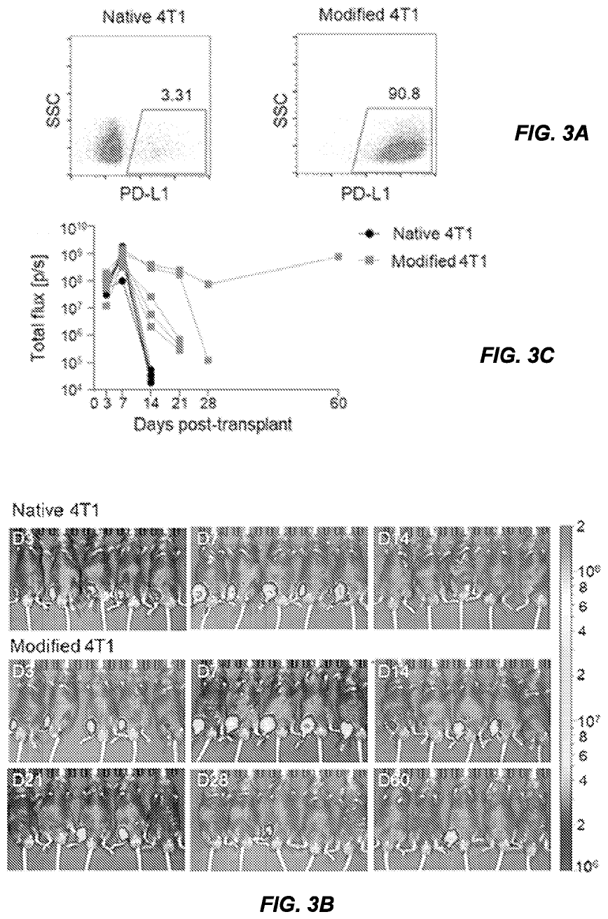

To evaluate whether the PD-L1 and CTLA4-Ig expression itself can protect allogeneic cells from immune rejection, 4T1 cells from BALB/c mice were transfected with two lentivirus vectors carrying green fluorescent protein (GFP)/luciferase and PD-L1/CTLA4-Ig, respectively. The GFP/luciferase 4T1 cells were first generated and then modified with PD-L1/CTLA4-Ig. Flow cytometry revealed that around 91% of modified cells overexpressed PD-L1, while only 3% of native 4T1 cells had PD-L1 expression (FIG. 3A). The GFP/luciferase-expressing 4T1 cells, with or without PD-L1/CTLA4-Ig expression, were transplanted in the right hindlimbs of healthy allogeneic C57BL/6 mice, and their survival was compared through bioluminescent imaging. The results showed that 4T1 cells without PD-L1/CTLA4-Ig expression were rejected within 14 days after transplantation in all C57BL/6 recipients (five of five), while PD-L1/CTLA4-Ig-expressing 4T1 cells survived in four of five animals at 14 days after transplantation, with one that survived for as long as 60 days (FIGS. 3B, 3C). The survival curve indicated that expression of PD-L1 and CTLA4-Ig significantly delayed allorejection (FIG. 3D) with a median survival time of 21 days (P<0.05).

A 4T1 tumor was formed in the hindlimb of allogeneic mouse 60 days after the injection of modified 4T1 cells (FIG. 3E). As control, GFP/luciferase-expressing native 4T1 cells were injected into the hindlimb of syngeneic BALB/c mice, which also formed a tumor (FIG. 3E). The morphology of the 4T1 tumor engrafted in the allogeneic C57BL/6 mouse was similar to that of the tumor formed by native 4T1 cells in the syngeneic BALB/c mouse at 60 days (FIG. 3E). Immunofluorescent staining showed that most of the cells within the modified 4T1 allograft were stained with PD-L1, while very few cells within the native 4T1 syngeneic graft expressed PD-L1 (FIG. 3F). Both the allograft and syngeneic graft cells were isolated from the tumor and further analyzed by flow cytometry. Not unexpectedly, 90.3% of engrafted cells expressed PD-L1, while only 0.8% of native 4T1 cells were positive with PD-L1 expression (FIG. 3G). Together, these data demonstrated that the expression of PD-L1 and CTLA4-Ig delayed allorejection.

### Example 3—PD-L1/CTLA4-Ig-overexpressing MSCs Improve Syngeneic Islet Transplantation

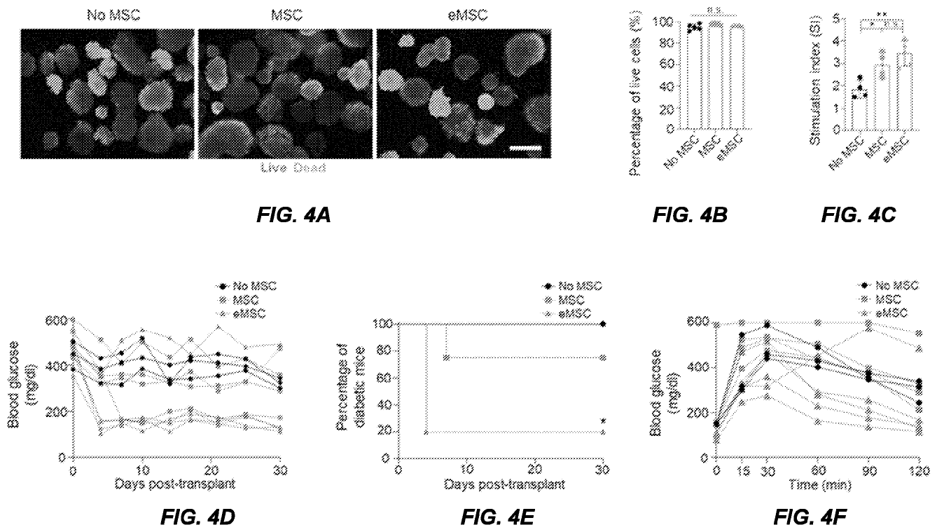

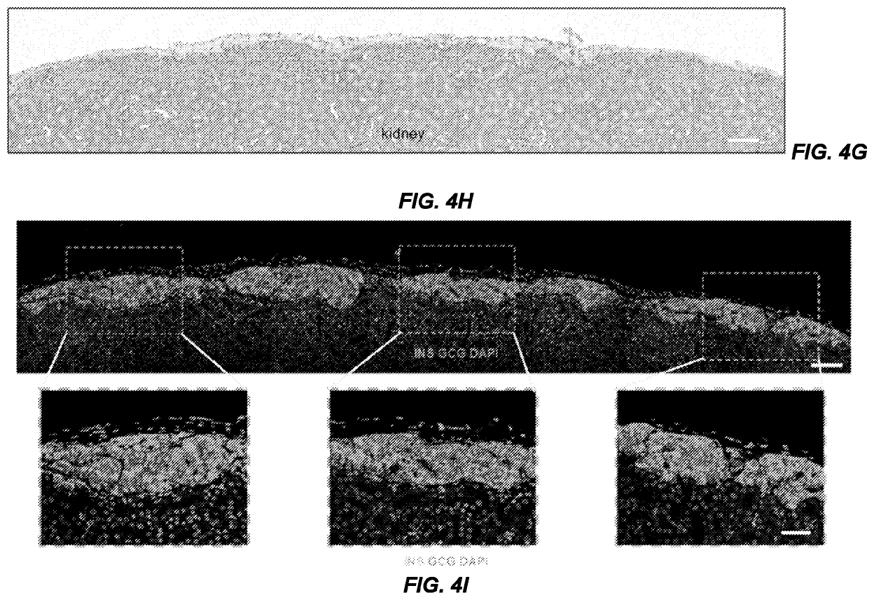

After confirming the immunomodulatory effects of PD-L1/CTLA4-Ig expression in allogeneic cells, the therapeutic potential of the eMSCs as protective accessory cells in islet transplantation was explored. First, the in vitro biocompatibility of the eMSCs with islets was examined by coculturing mouse islets with MSCs or eMSCs for 24 hours, using islets cultured alone as control. The live and dead imaging and quantitative analysis of fluorescence intensity demonstrated that there was no difference in terms of the viability of the islets among the three groups (FIGS. 4A, 4B). Furthermore, to test the function of islets after coculture, the glucose-stimulated insulin secretion (GSIS) assay was carried out in vitro. Islets from all groups responded to low and high glucose and secreted more insulin at high glucose than at low glucose while maintaining their capability to shut down insulin secretion during a second low-glucose treatment. The stimulation index (“SI”, which is the ratio of insulin secretion at high glucose to that at low glucose) of islets cocultured with MSCs or eMSCs was significantly higher than that of islets cultured alone (P<0.05) (FIG. 4C). This was likely caused by the beneficial paracrine effects between the MSCs and the islets (Rackham et al., “Annexin A1 is a Key Modulator of Mesenchymal Stromal Cell-mediated Improvements in Islet Function,” Diabetes 65:db150990 (2015); Jung et al., “Bone Marrow-derived Mesenchymal Stromal Cells Support Rat Pancreatic Islet Survival and Insulin Secretory Function in vitro,” Cytotherapy 13:19-29 (2011); Ito et al., “Mesenchymal Stem Cell and Islet Co-transplantation Promotes Graft Revascularization and Function,” Transplantation 89:1438-1445 (2010); Yoshimatsu et al., “The Co-transplantation of Bone Marrow-derived Mesenchymal Stem Cells Reduced Inflammation in Intramuscular Islet Transplantation,” PLOS ONE 10:e0117561 (2015); Cai et al., “Umbilical Cord Mesenchymal Stromal Cell With Autologous Bone Marrow Cell Transplantation in Established Type 1 Diabetes: A Pilot Randomized Controlled Open-Label Clinical Study to Assess Safety and Impact on Insulin Secretion,” Diabetes Care 39:149-157 (2015), each of which is hereby incorporated by reference in its entirety).

Then, the persistence of MSC single cells and MSC spheroids was investigated in vivo. The GFP/luciferase MSCs were generated as reported previously (Wang et al., “A Nanofibrous Encapsulation Device for Safe Delivery of Insulin-producing Cells to Treat Type 1 Diabetes,” Sci. Transl. Med. 13:eabb4601 (2021), which is hereby incorporated by reference in its entirety). A total of 500 to 600 GFP/luciferase MSC spheroids and the corresponding number of GFP/luciferase MSC single cells were transplanted under the kidney capsule of healthy C57BL/6 mice. The whole-body images showed that no bioluminescent signals were detected after 14 days in mice receiving MSC single cells, while MSC spheroids survived as long as 30 days. Thus, MSC spheroids were chosen for subsequent experiments rather than MSC single-cell suspension, because of the improved survival in vivo.

Next, a marginal dose [150 to 200 islet equivalent (IEQ)] of syngeneic islets was cotransplanted without MSCs (no-MSC group), with (500 to 600) MSC spheroids (MSC group), or with (500 to 600) eMSC spheroids (eMSC group) in the kidney capsule of streptozotocin (STZ)-induced C57BL/6 diabetic mice. The dosage of MSC spheroids was determined on the basis of the balance of having enough eMSCs to protect allogeneic islets while not occupying too much space and competing for oxygen and nutrients with islets. Non-fasting blood glucose curves showed that none of the mice in the no-MSC group and one of four mice in the MSC group became normoglycemic after transplantation (FIG. 4D). However, four of five mice reversed diabetes 4 days after transplantation in the eMSC group (FIG. 4D). The curve of diabetic mice percentage demonstrated that eMSCs significantly (P<0.05) improved syngeneic islet engraftment with a median time to cure of 4 days and reduced islet number needed for diabetes correction (FIG. 4E). The intraperitoneal glucose tolerance test (IPGTT) performed on mice with different grafts on day 30 showed that four engrafted mice receiving islets with eMSCs and one engrafted mouse receiving islets with MSCs cleared blood glucose within 2 hours after injection (FIG. 4F); all other recipients failed to achieve metabolic control over glucose (FIG. 4F). The hematoxylin and eosin (H&E) and immunofluorescent staining showed the engraftment of islets cotransplanted with eMSCs in the kidney capsule and the expression of insulin and glucagon within the islets 30 days after transplantation (FIG. 4G-4I). In contrast, islets without MSCs or with native MSCs were found to be less maintained in the kidney capsule as shown by H&E staining. The improved outcome by the eMSCs may be due to their anti-inflammatory function, as discussed infra.

### Example 4—PD-L1/CTLA4-Ig-Overexpressing MSCs Improve Allogeneic Islet Transplantation

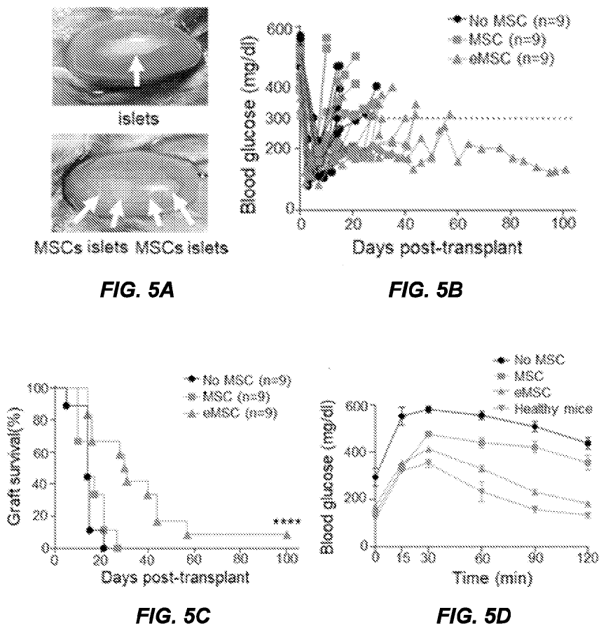

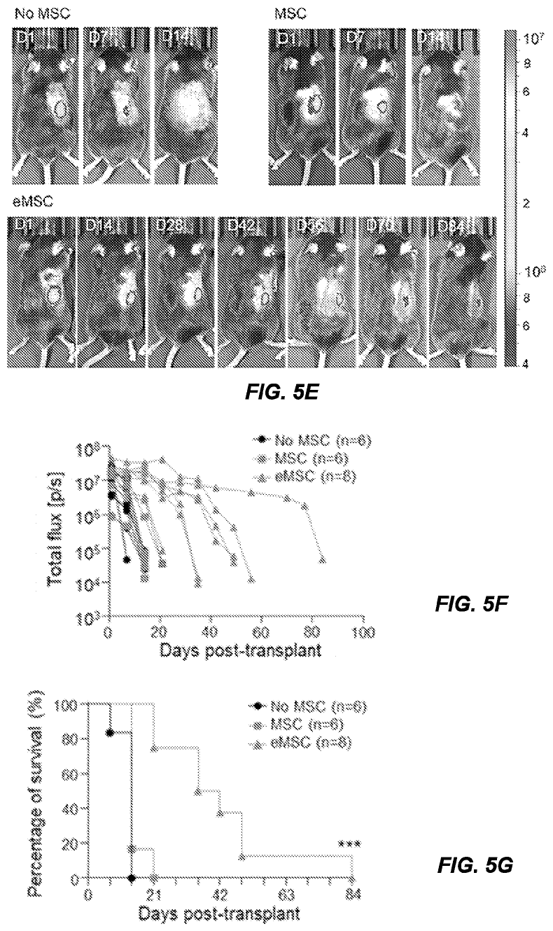

A more clinically relevant application of immunoprotective accessory cells such as the eMSCs would be for allogeneic transplantation. Therefore, BALB/c mouse islets were cotransplanted with eMSCs into diabetic C57BL/6 mice to test whether they could delay allorejection. Islets transplanted without MSCs (no-MSC group) or with native MSCs (MSC group) were included as control. In each mouse, around 500 to 600 IEQ BALB/c mouse islets were transplanted in one of the kidney capsules (FIG. 5A). The blood glucose curves showed that BALB/c islets in the no-MSC group or MSC group were all rejected within 14 and 20 days, respectively (FIGS. 5B, 5C). In contrast, mice transplanted with islets and 500 to 600 eMSC spheroids maintained normoglycemia for as long as 100 days (FIGS. 5B, 5C). Quantitative analysis showed that allogeneic islets cotransplanted with eMSCs survived significantly longer than the other two groups (P<0.0001) (FIG. 5C). The median survival times of these three groups were 14 days (no MSC), 14 days (MSC), and 40 days (eMSC) (n=9), indicating that the eMSCs significantly delayed allorejection and prolonged allograft survival compared to other groups (P<0.0001) (FIGS. 5B, 5C). The IPGTT performed on mice with different grafts on day 30 showed that mice receiving islets with eMSCs cleared blood glucose within 2 hours after injection, similar to the healthy mice, while those receiving islets without MSCs or with MSCs failed to achieve metabolic control over glucose (FIG. 5D).

To continuously and more directly monitor islet survival in vivo, around 500 IEQ GFP/luciferase expressing friend virus B (FVB) mouse islets were transplanted in the kidney capsule of diabetic C57BL/6 mice. The bioluminescent imaging showed that the signal was localized in the kidney, and allogeneic islets without MSCs were rejected within 14 days (FIG. 5E). Although better islet survival was observed within 7 days when islets were cotransplanted with MSCs compared to the no-MSC group, the islets were eventually rejected within 14 days (FIGS. 5E, 5F). However, the bioluminescent signal lasted for as long as 70 days when islets were cotransplanted with the eMSCs, and they were not completely rejected until day 84 (FIGS. 5E, 5F). Quantitative analysis confirmed that the eMSCs significantly improved allogeneic islet survival compared to no-MSC and MSC groups (P<0.001) (FIGS. 5F, 5G).

The next experiment assessed whether the immunomodulatory effects of the eMSCs are only local and not systemic. To answer this question, the systemic immune response was investigated in the spleen of diabetic C57BL/6 mice receiving allogeneic islets with/without MSCs or with eMSCs on day 14 after transplantation. The data indicated that CD3⁺ T cells and PD-1⁺ cells were observed in the spleen in all three groups with an average density of 10,000 and 15,000 cells/mm², respectively. There was no difference among the three groups in terms of the CD3⁺ T cell and PD-1⁺ cell densities (P>0.05). Besides this, ˜500 to 600 IEQ BALB/c islets and 500 to 600 eMSC spheroids were transplanted separately into two kidneys in the same C57BL/6 mouse recipient with diabetes. The blood glucose curves showed that islets were rejected in all the recipients within 14 days, and the eMSCs did not have any protective effects on allogeneic islets that were transplanted in different kidney capsules (FIG. 8). Together, the data confirm that the immunomodulation by the eMSCs is a local effect.

### Example 5—PD-L1/CTLA4-Ig-Overexpressing MSCs Promote Immune Tolerant Microenvironment

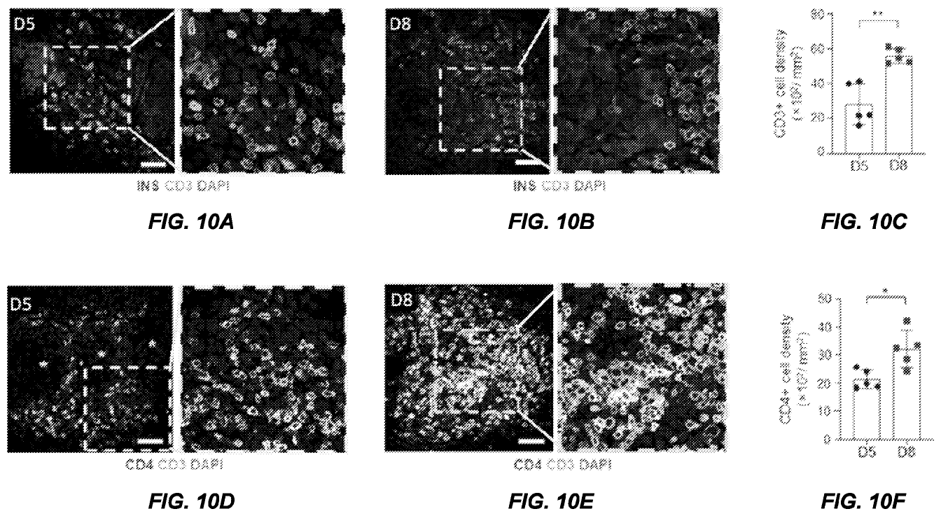

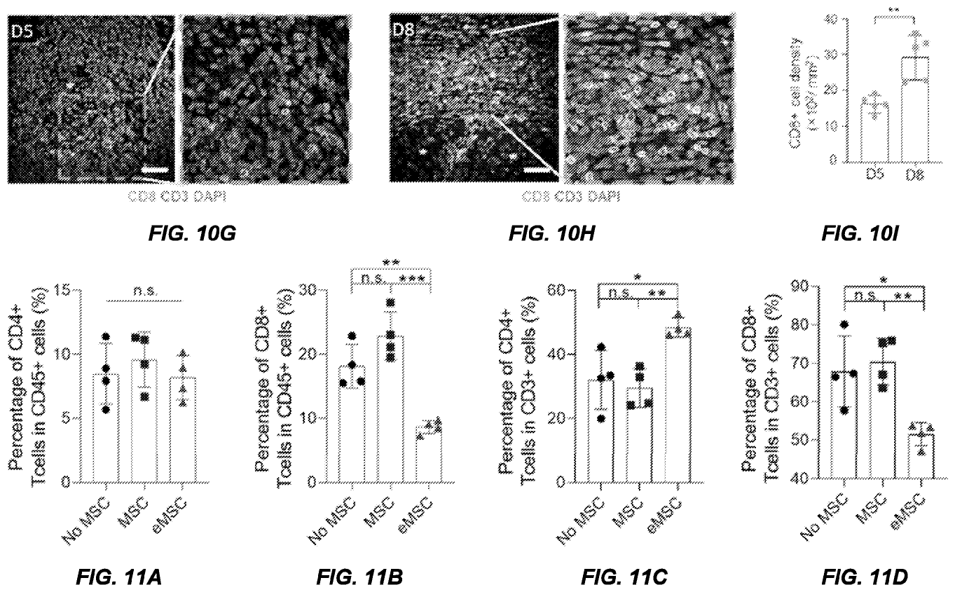

To understand how the eMSCs protected the islets from allorejection, the progression of immune response was examined in allogeneic islet grafts with no MSCs. Grafts were retrieved on days 5, 8, and 15 and analyzed by H&E and immunofluorescent staining. Histological images demonstrated that host immune cells infiltrated the allograft on day 5, accumulated around the islets on day 8, and, lastly, replaced islet cells on day 15 (FIGS. 9A-9C). Immunofluorescent staining images of grafts showed that insulin⁺ β cells were surrounded and infiltrated by host CD3⁺ T cells (FIGS. 10A, 10B). The ratio of CD3⁺ T cells to insulin+β cells in allograft retrieved on day 8 was significantly higher than that on day 5 (P<0.01), indicating progressive T cell infiltration overtime (FIGS. 10A-10C). Costaining of CD3 with CD4 or CD3 with CD8 demonstrated that the infiltrated T cells were CD4⁺ or CD8⁺ T cells (FIGS. 10D-10I) and both types of cells increased in number significantly from day 5 to day 8 (P<0.05), a time point chosen for the immune profiling in all the groups.

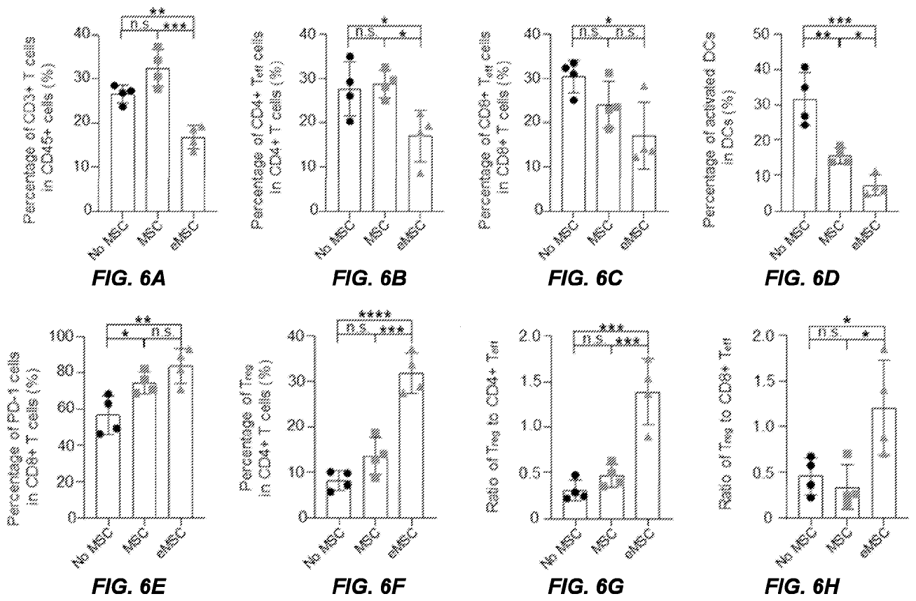

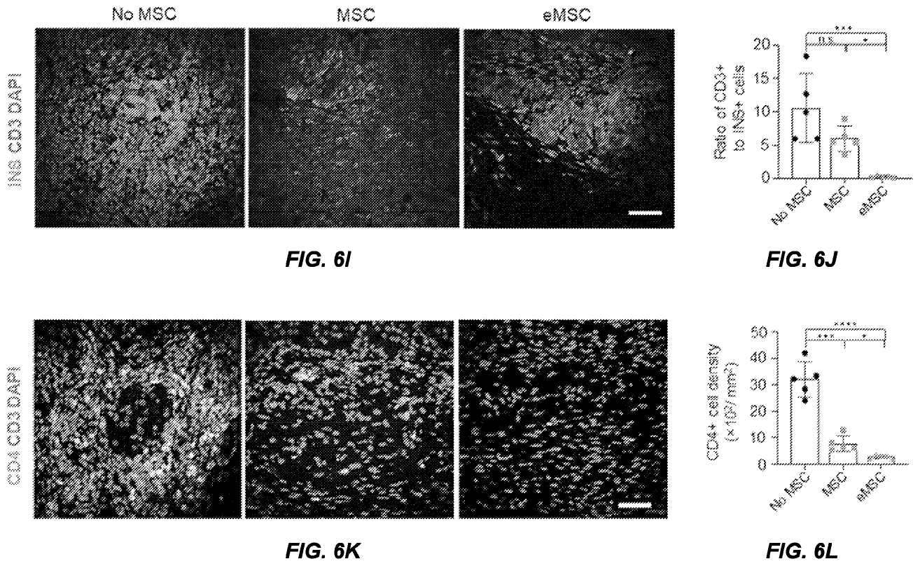

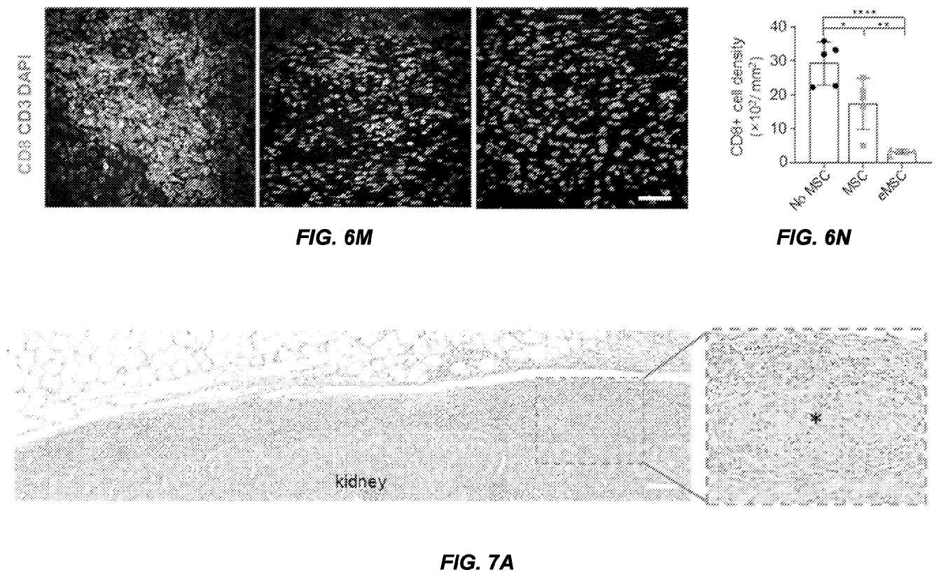

Next, the immune cells at the graft site were characterized in response to the PD-L1/CTLA4-Ig presentation by transplanting allogeneic islets (500 IEQ) without any MSCs or with either MSCs or eMSCs into diabetic C57BL/6 mice. On day 8 after transplantation, the grafts were explanted and the cells within the graft tissues were isolated. Flow cytometry analysis was carried out to identify the immune cell population based on the expression of markers including CD45, CD3, CD4, CD8, CD25, CD44, CD62L, CD11c, CD86, Foxp3, and PD-1. Results showed that the percentage of CD3⁺ T cells in eMSC grafts was significantly lower than those in no-MSC grafts and MSC grafts (P<0.01) (FIG. 6A). Although there was no difference in terms of the CD4⁺ T cell percentage in CD45⁺ cell population among different groups (P>0.05), the CD8⁺ T cells in the CD45⁺ cell population were significantly fewer in the eMSC group compared to the other two groups (P<0.01) (FIGS. 11A, 11B). In addition, more CD4⁺ T cells and fewer CD8⁺ T cells were observed within the CD3⁺ T cell population in the eMSC group compared to the other two groups (P<0.05) (FIGS. 11C, 11D). Furthermore, the percentage of CD4⁺ and CD8⁺ T_{eff} cells (CD44^{hi}CD62L^{lo}) and activated dendritic cells (CDI 1c+CD86*) also decreased significantly in eMSC grafts compared to the other groups (P<0.05) (FIGS. 6B-6D). It was additionally found that the percentage of CD8⁺ T cells expressing an exhaustion/anergy marker such as PD-1 increased significantly in the eMSC group compared to the no-MSC group (P<0.01) (FIG. 6E). Further investigation revealed that the percentage of CD4⁺ T_{reg} cells (CD4⁺ CD25⁺ Foxp3⁺) (FIG. 6F) and the ratio of CD4⁺ T_{reg} cells to CD4⁺ or CD8⁺ T_{eff} cells (FIGS. 6G, 6H) in the eMSC group were significantly higher than those in the other two groups (P<0.05).

Histological analysis of different grafts retrieved on day 8 corroborated the trends observed from flow cytometry. Specifically, immunofluorescent staining showed that the density of CD3⁺ T cells and the ratio of CD3⁺ T cells to insulin⁺β cells decreased significantly in allografts cotransplanted with eMSCs than those without MSCs or with MSCs (P<0.05) (FIGS. 6I, 6J). The densities of CD4⁺ and CD8⁺ T cells in the grafts cotransplanted with eMSCs were also significantly lower than those in the other two groups (P<0.05) (FIGS. 6K-6N).

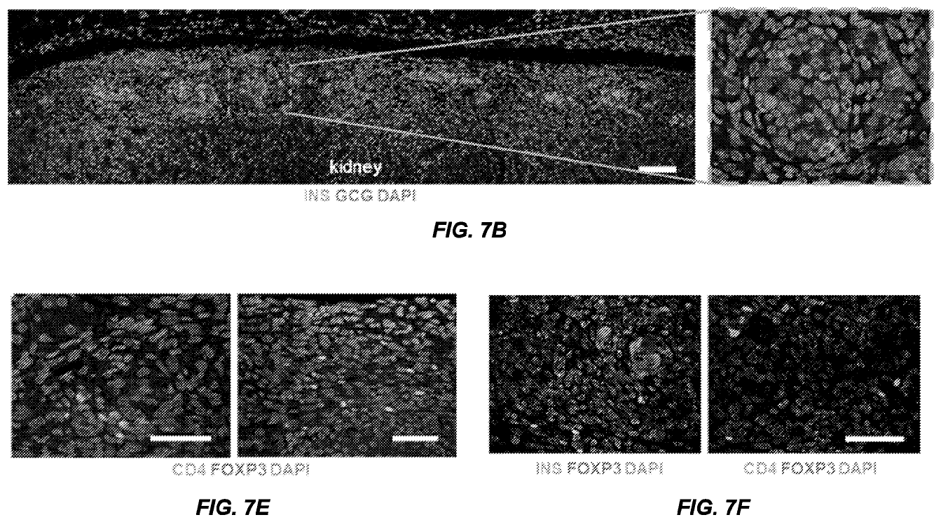

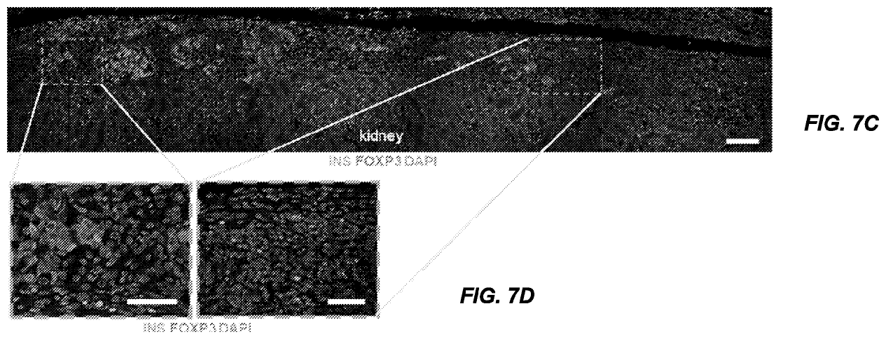

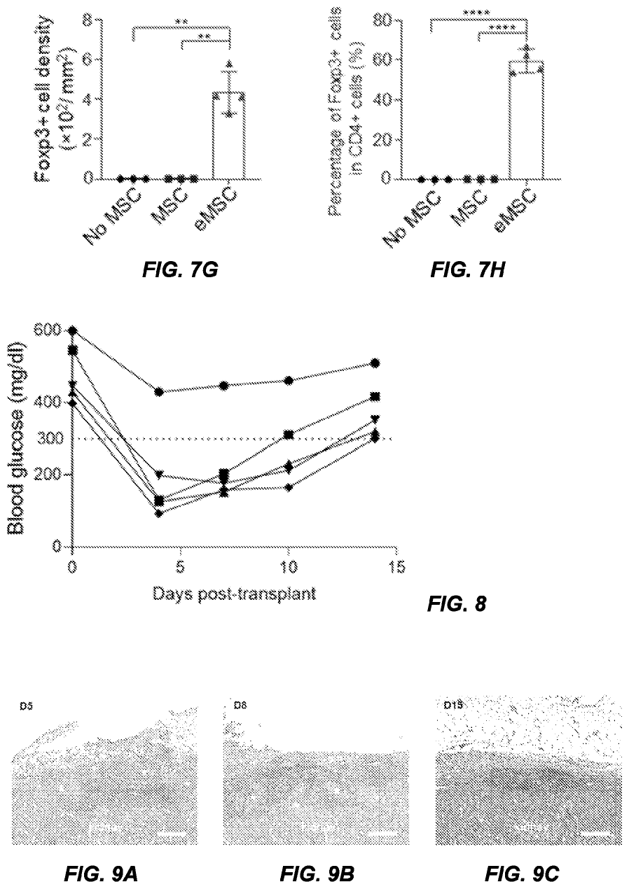

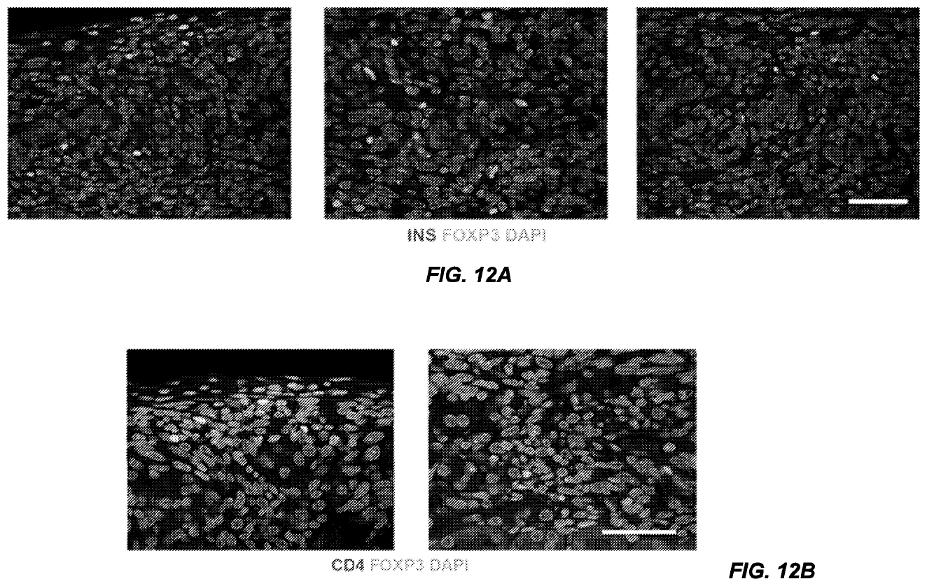

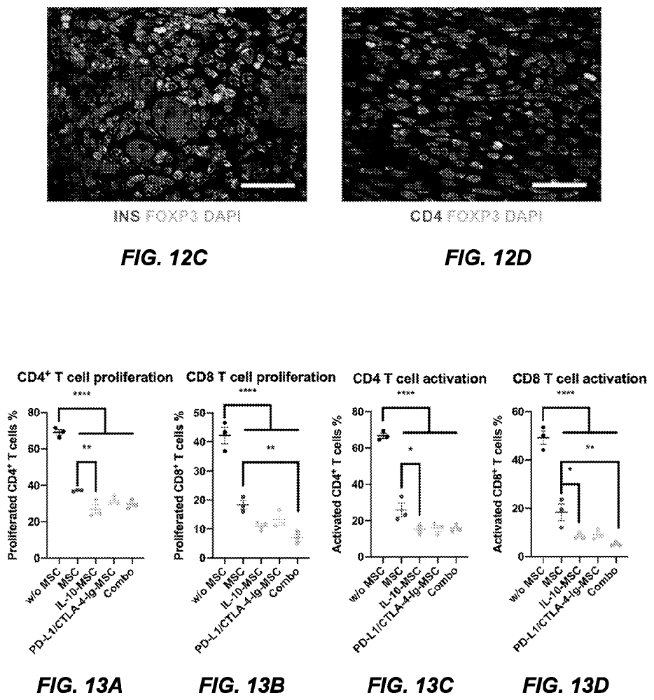

The grafts were also analyzed after a longer-term transplantation. The H&E image showed that allogeneic islets were maintained in the kidney capsule after 45 days' transplantation (FIG. 7A). Immunofluorescent staining for insulin and glucagon demonstrated that cells maintained their individual hormone identities, and many insulin-expressing p cells survived in the allogeneic mice (FIG. 7B). Costaining of insulin and Foxp3 showed that insulin⁺β cells were surrounded by many Foxp3⁺ T_{reg} cells (FIGS. 7C, 7D, 12A, 12C), which might be responsible for the long-term survival of allogeneic islets in vivo. The Foxp3⁺ T_{reg} cells within the grafts were further confirmed to express CD4 marker by costaining of CD4 and Foxp3 (FIGS. 7E, 12B, 12D). Furthermore, examination of the retrieved grafts 103 days after transplantation showed the survival of insulin⁺β cells surrounded by CD4⁺ Foxp3⁺ T_{reg} cells (FIG. 7F, 12C). Quantitative analysis showed that the density of Foxp3⁺ T_{reg} cells in the eMSC graft in the long term was around 450/mm², while there was no Foxp3⁺ T_{reg} cells found in the eradicated allograft in the other two groups (FIG. 7G). The percentage of Foxp3⁺ T_{reg} cells in the CD4⁺ T cells was around 60% within the eMSC graft, which was significantly higher than that in no-MSC and MSC grafts (P<0.0001) (FIG. 7H). Together, these data showed that the eMSCs suppressed CD4⁺ and CD8⁺ T_{eff} cells and promoted CD4⁺ T_{reg} cells within the graft. This tolerant immune microenvironment may be responsible for the delayed allorejection and prolonged islet survival.

### Discussion of Examples 1-5

Islet transplantation offers T1D patients many benefits including improved glucose control, prevention of dangerous hypoglycemia unawareness, and reduced risks of diabetes-related complications. However, chronic systemic immunosuppression required to prevent immune rejection can affect the longevity of the implanted islets and trigger adverse side effects on patients such as infections and cancer. In this study, we set out to develop a type of immunoprotective accessory cells that can be mixed and cotransplanted with islets using established clinical procedures but with no or reduced systemic immunosuppression.

MSCs were selected as the starting cells for multiple reasons. They exist in many tissues, can be obtained by isolating fat tissues with minimally invasive surgery, have been widely used in clinical applications for tissue regeneration, and have an acceptable safety profile (Bianco et al., “The Meaning, the Sense and the Significance: Translating the Science of Mesenchymal Stem Cells into Medicine,” Nat. Med. 19:35-42 (2013); Uccelli et al., “Mesenchymal Stem Cells in Health and Disease,” Nat. Rev. Immunol. 8:726-736 (2008), each of which is hereby incorporated by reference in its entirety). To date, more than 1000 clinical trials exploring MSCs are registered by the U.S. Food and Drug Administration and many have demonstrated that MSCs can be safely infused even in high doses in patients (Levy et al., “Shattering Barriers Toward Clinically Meaningful MSC Therapies,” Sci. Adv. 6:eaba6884 (2020); Lalu et al., Canadian Critical Care Trials Group, “Safety of Cell Therapy with Mesenchymal Stromal Cells (SafeCell): A Systematic Review and Meta-analysis of Clinical Trials,” PLOS ONE 7:e47559 (2012), each of which is hereby incorporated by reference in its entirety). For example, in a phase 1/2 trial (TREAT-ME1, Trial NCT02008539), which involved intravenous administration of autologous MSCs engineered to express the tumor-specific herpes simplex virus-thymidine kinase gene to treat gastrointestinal tumors, investigators reported favorable safety in patients who received the treatment (Niess et al., “Treatment of Advanced Gastrointestinal Tumors with Genetically Modified Autologous Mesenchymal Stromal Cells (TREAT-ME1): Study Protocol of a Phase I/II Clinical Trial,” BMC Cancer 15:237 (2015), which is hereby incorporated by reference in its entirety). Previous studies have also shown that MSCs infused with islets into the hepatic portal vein improved syngeneic rat islet (Ito et al., “Mesenchymal Stem Cell and Islet Co-transplantation Promotes Graft Revascularization and Function,” Transplantation 89:1438-1445 (2010), which is hereby incorporated by reference in its entirety) engraftment and rat and human islet (Forbes et al., “Human umbilical Cord Perivascular Cells Improve Human Pancreatic Islet Transplant Function by Increasing Vascularization,” Sci. Transl. Med. 12:eaan5907 (2020), which is hereby incorporated by reference in its entirety) engraftment in immunodeficient mice and promoted vascularization by secreting paracrine factors (Bianco et al., “The Meaning, the Sense and the Significance: Translating the Science of Mesenchymal Stem Cells into Medicine,” Nat. Med. 19:35-42 (2013); Ito et al., “Mesenchymal Stem Cell and Islet Co-transplantation Promotes Graft Revascularization and Function,” Transplantation 89:1438-1445 (2010); Cai et al., “Umbilical Cord Mesenchymal Stromal Cell With Autologous Bone Marrow Cell Transplantation in Established Type 1 Diabetes: A Pilot Randomized Controlled Open-Label Clinical Study to Assess Safety and Impact on Insulin Secretion,” Diabetes Care 39:149-157 (2015); Uccelli et al., “Mesenchymal Stem Cells in Health and Disease,” Nat. Rev. Immunol. 8:726-736 (2008); Forbes et al., “Human umbilical Cord Perivascular Cells Improve Human Pancreatic Islet Transplant Function by Increasing Vascularization,” Sci. Transl. Med. 12:eaan5907 (2020), each of which is hereby incorporated by reference in its entirety). To create the eMSCs with immunoprotective function, PD-L1 was expressed on their surface and CTLA4-Ig was expressed as an extracellularly released factor. PD-1 (CD279) and CTLA-4 (CD152) are two critical and potent regulators of peripheral T cell tolerance and T cell function (Bluestone et al., “CTLA4Ig: Bridging the Basic Immunology with Clinical Application,” Immunity 24:233-238 (2006); Chen et al., “Elements of Cancer Immunity and the Cancer-immune Set Point,” Nature 541:321-330 (2017), each of which is hereby incorporated by reference in its entirety); these two signaling pathways have been used in modulating alloreactive responses in multiple transplantation models. Engineering of primary islets with PD-L1 and/or CTLA4-Ig by gene modification or protein conjugation resulted in survival of islet allograft for more than 100 days (Batra et al., “Localized Immunomodulation with PD-L1 Results in Sustained Survival and Function of Allogeneic Islets Without Chronic Immunosuppression,” J. Immunol. 204:2840-2851 (2020); Li et al., “PD-Li-driven tolerance protects Neurogenin 3-induced Islet Neogenesis to Reverse Established Type 1 Diabetes in NOD Mice,” Diabetes 64:529-540 (2014), each of which is hereby incorporated by reference in its entirety). Knocking in human PD-L1 and CTLA4-Ig to human embryonic stem cells protected them from allogeneic immune responses in humanized mice (Rong et al., “An Effective Approach to Prevent Immune Rejection of Human ESC-derived Allografts,” Cell Stem Cell 14:121-130 (2014), which is hereby incorporated by reference in its entirety). Overexpression of PD-L1 protected stem cell-derived islet organoids in diabetic xenogeneic mice and allogeneic humanized mice and restored glucose homeostasis for 50 and 25 days, respectively (Yoshihara et al., “Immune-evasive Human Islet-like Organoids Ameliorate Diabetes,” Nature 586:606-611 (2020), which is hereby incorporated by reference in its entirety). Despite these advances, manipulation of islets can be laborious and negatively affect their function, and generation of hypoimmunogenic cells from stem cells may cause safety concerns (González et al., “How Safe Are Universal Pluripotent Stem Cells?” Cell Stem Cell 26:307-308 (2020), which is hereby incorporated by reference in its entirety). Therefore, accessory cells such as the eMSCs we created in this study may provide an easier and safer way to circumvent the need for chronic systemic immunosuppression in islet transplantation.

The eMSCs had similar gene expressions to the MSCs except for the exogenous PD-L1/CTLA4-Ig genes that were purposely introduced and had overexpression by hundred-fold. We used MSC spheroids to cotransplant with islets instead of using single cells because MSC spheroids showed improved survival compared to MSC single cells in kidney capsule. In addition, previous studies showed elevated gene expressions associated with anti-apoptotic factors such as B cell lymphoma extra large (Bcl-xL) and pro-angiogenesis factors such as vascular endothelial growth factor, fibroblast growth factor-2, and hepatocyte growth factor in spheroids compared to the cells in the two-dimensional monolayer culture (Wang et al., “The Paracrine Effects of Adipose-derived Stem Cells on Neovascularization and Biocompatibility of a Macroencapsulation Device,” Acta Biomater. 15:65-76 (2015); Bhang et al., “Angiogenesis in Ischemic Tissue Produced by Spheroid Grafting of Human Adipose-derived Stromal Cells,” Biomaterials 32:2734-2747 (2011), each of which is hereby incorporated by reference in its entirety). Both the MSC or eMSC spheroids were compatible with islets in vitro and improved their glucose-stimulated insulin secretion after coculture with islets. Furthermore, the eMSCs were shown to be able to suppress T cell proliferation, activation, and cytotoxicity of allogeneic and diabetogenic CD4⁺ and CD8⁺ in vitro in a mixed lymphocyte reaction in the presence of allogeneic antigen-presenting cells. The in vitro results indicate that eMSCs could not only protect islets immunologically by inhibiting T_{eff} cells, but also enhance their function by paracrine factors.

The therapeutic potential of the eMSCs was first demonstrated in a syngeneic islet transplantation model. Autologous islet transplantation is a clinically proven treatment option for patients with chronic pancreatitis who undergo pancreatectomy. However, even in the absence of immune rejection, the early posttransplant inflammation can cause extensive β cell loss (Stabler et al., “Engineering Immunomodulatory Biomaterials for Type 1 Diabetes,” Nat. Rev. Mater. 4:429-450 (2019); Brusko et al., “Strategies for Durable β Cell Replacement in Type 1 Diabetes,” Science 373:516-522 (2021), each of which is hereby incorporated by reference in its entirety). Therefore, inhibition of early inflammatory events is expected to improve long-term islet function. The signaling pathway of PD-L1 and CTLA4 can regulate not only alloresponses but also inflammatory responses. The up-regulation of PD-L1 expression in response to inflammatory cytokines acts as a natural “balance” to limit tissue-specific responses to inflammation (Keir et al., “PD-1 and Its Ligands in Tolerance and Immunity,” Annu. Rev. Immunol. 26:677-704 (2008); Yamazaki et al., “Expression of Programmed Death 1 Ligands by Murine T Cells and APC,” J. Immunol. 169:5538-5545 (2002), each of which is hereby incorporated by reference in its entirety). For example, genetically modified PD-L1-overexpressing dendritic cells differentiated from mouse embryonic stem (ES) cells were demonstrated to limit spinal cord inflammation (Hirata et al., “Prevention of Experimental Autoimmune Encephalomyelitis by Transfer of Embryonic Stem Cell-derived Dendritic Cells Expressing Myelin Oligodendrocyte Glycoprotein Peptide Along with TRAIL or Programmed death-1 Ligand,” J. Immunol. 174:1888-1897 (2005), each of which is hereby incorporated by reference in its entirety). Similarly, CTLA-4 signaling also helps bring an inflammatory response back down to homeostatic levels (Bluestone et al., “CTLA4Ig: Bridging the Basic Immunology with Clinical Application,” Immunity 24:233-238 (2006), which is hereby incorporated by reference in its entirety). For example, CTLA4-Ig therapy can reduce joint inflammation and damage in patients with active rheumatoid arthritis, which is a systemic inflammatory disorder (Maxwell et al., “Abatacept for Rheumatoid Arthritis,” Cochrane Database Syst. Rev. 2009, CD007277 (2009), which is hereby incorporated by reference in its entirety). Thus, the observed improvement of syngeneic islet transplantation using eMSCs with PD-L1 and CTLA4-Ig expression might be explained by the anti-inflammatory properties of these two ligands. The eMSCs may offer a new option to mitigate the early inflammation and improve autologous islet transplantation.

The eMSCs were also shown to improve the allogeneic islet transplantation in a diabetic mouse model. Without any immunosuppression, the eMSCs delayed allograft failure significantly, as evidenced by both blood glucose monitoring and bioluminescent imaging. Although the kidney capsule transplantation used in this study is different from the portal vein transplantation in clinical practice, it is possible that the eMSCs may be mixed with islets and cotransplanted into the portal vein (Forbes et al., “Human Umbilical Cord Perivascular Cells Improve Human Pancreatic Islet Transplant Function by Increasing Vascularization,” Sci. Transl. Med. 12:eaan5907 (2020); Wang et al., “Autologous Mesenchymal Stem Cell and Islet Cotransplantation: Safety and Efficacy,” Stem Cell Transl. Med. 7:11-19 (2017), each of which is hereby incorporated by reference in its entirety). Therefore, future studies may be directed at testing whether eMSCs can be used in portal vein and eventually clinical islet transplantation and improve the therapeutic outcome with reduced systemic immunosuppression. We analyzed local immune responses in the eMSC/islet graft at different time points using flow cytometry and immunostaining. The results showed the eMSCs suppressed host CD4⁺ and CD8⁺ T_{eff} cell activation, induced T cell exhaustion, and promoted T_{reg} cells, all of which could be responsible for the observed delay of allorejection.

Pioneering work has been done recently on synthetic biomaterial platforms for local immunomodulation for islet transplantation. For example, PEG microgels or poly(lactide-co-glycolide) (PLG) scaffold were modified to display PD-L1 and Fas ligand (FasL) through a streptavidin/biotin interaction (Coronel et al., “Immunotherapy via PD-L1-presenting Biomaterials Leads to Long-term Islet Graft Survival,” Sci. Adv. 6:eaba5573 (2020); Headen et al., “Local Immunomodulation with Fas Ligand-engineered Biomaterials Achieves Allogeneic Islet Graft Acceptance,” Nat. Mater. 17:732-739 (2018); Skoumal et al., “Localized Immune Tolerance from FasL-functionalized PLG Scaffolds,” Biomaterials 192:271-281 (2018), each of which is hereby incorporated by reference in its entirety). Long-term (>100 days) survival of allogeneic islets was achieved using biomaterial approaches combined with a short course of rapamycin treatment. Synthetic biomaterials allow islet transplantation at extrahepatic sites such as omentum, but they can cause foreign body responses, induce antibodies, and may be challenging to be used in current clinical protocol, i.e., portal vein transplantation. In contrast, accessory cells such as the eMSCs can be obtained from autologous source, secrete a wide spectrum of beneficial paracrine factors, and be mixed with islets and transplanted with no or minimal modifications of current clinical procedures. In addition, factors delivered or presented by biomaterials may be depleted or degraded over time. The accessory cells on the other hand act as a “living factory” to produce tolerogenic ligands, either immobilized on the cell surface or released to the local environment. The eMSCs enabled the allogenic islet survival for up to more than 100 days with no short-term treatment of rapamycin or other immunosuppressive drugs.

T_{reg} cells have been shown to play an important role in maintaining homeostasis and peripheral tolerance (Raffin et al., T_{reg} Cell-based Therapies: Challenges and Perspectives,” Nat. Rev. Immunol. 20:158-172 (2019); Ferreira et al., “Next-generation Regulatory T Cell Therapy,” Nat. Rev. Drug Discov. 18:749-769 (2019), each of which is hereby incorporated by reference in its entirety). Both systemic infusion of autologous T_{reg} cells (Bluestone et al., “Type 1 Diabetes Immunotherapy Using Polyclonal Regulatory T Cells,” Sci. Transl. Med. 7:315ra189 (2015); Marek-Trzonkowska et al., “Therapy of Type 1 Diabetes with CD4⁺ CD25^{high}CD127-Regulatory T Cells Prolongs Survival of Pancreatic Islets-Results of One Year Follow-up,” Clin. Immunol. 153:23-30 (2014), each of which is hereby incorporated by reference in its entirety) and cotransplantation of T_{reg} with islets (Graham et al., “PLG Scaffold Delivered Antigen-specific Regulatory T Cells Induce Systemic Tolerance in Autoimmune Diabetes,” Tissue Eng Part A. 19:1465-1475 (2013); Takemoto et al., “Coaggregates of Regulatory T Cells and Islet Cells Allow Long-term Graft Survival in Liver Without Immunosuppression,” Transplantation 99:942-947 (2015), each of which is hereby incorporated by reference in its entirety) have been investigated in treating diabetes and shown therapeutic effects in preclinical trials. However, the mass production of T_{reg} cells and the maintenance of their long-term function remain challenging. Thus, there are attempts to engineer other cell types with regulatory ligands. In one study, syngeneic myoblasts were edited to express FasL on the cell surface and protected islet allograft (Lau et al., “Prevention of Islet Allograft Rejection with Engineered Myoblasts Expressing FasL in Mice,” Science 273:109-112 (1996), which is hereby incorporated by reference in its entirety). However, myoblasts are relatively difficult to acquire noninvasively. In addition, while FasL induces T cell death (Motz et al., “Tumor Endothelium FasL Establishes a Selective Immune Barrier Promoting Tolerance in Tumorst,” Nat. Med. 20:607-615 (2014), which is hereby incorporated by reference in its entirety), it may also activate innate immune responses (Restifo, “Not So Fas: Re-evaluating the Mechanisms of Immune Privilege and Tumor Escape,” Nat. Med. 6:493-495 (2000), which is hereby incorporated by reference in its entirety). In another study, researchers engineered syngeneic fibroblasts using adenovirus to overexpress indoleamine 2,3 dioxygenase (IDO) and achieved allograft survival in IDO-expressing composite up to 51 days (Jalili et al., “Local Expression of Indoleamine 2,3 Dioxygenase in Syngeneic Fibroblasts Significantly Prolongs Survival of an Engineered Three-dimensional Islet Allograft,” Diabetes 59:2219-2227 (2010), which is hereby incorporated by reference in its entirety). Although IDO degrades tryptophan required for T cell growth and suppresses T cell responses (Chen et al., “IDO: More than an Enzyme,” Nat. Immunol. 12:809-811 (2011); Mellor et al., “Indoleamine 2,3 Dioxygenase and Regulation of T Cell Immunity,” Biochem. Biophys. Res. Commun. 338:20-24 (2005), each of which is hereby incorporated by reference in its entirety), the potency of IDO pathway may be lower compared to PD-L1 (Chinn et al., “PD-L1 and IDO Expression in Cervical and Vulvar Invasive and Intraepithelial Squamous Neoplasias: Implications for Combination Immunotherapy,” Histopathology 74:256-268 (2019); Zhang et al., “Differential Expression of PD-L1 and IDO1 in Association with the Immune Microenvironment in Resected Lung Adenocarcinomas,” Mod. Pathol. 32:511-523 (2019), each of which is hereby incorporated by reference in its entirety). Nevertheless, CTLA4-Ig has been reported to trigger the production of IDO in APCs (Grohmann et al., “CTLA-4-Ig Regulates Tryptophan Catabolism in vivo,” Nat. Immunol. 3:1097-1101 (2002), which is hereby incorporated by reference in its entirety), suggesting multiple ways in which immune regulation following CTLA4-Ig treatment may occur. Furthermore, in both studies, the immunomodulatory ligands were either immobilized on the cell surface or released to the peripheral environment. In the eMSCs we described here, the PD-L1 was immobilized on the cell surface and CTLA4-Ig was released. This dual modulation approach suppresses T cell function in a nonredundant way (Curran et al., “PD-1 and CTLA-4 Combination Blockade Expands Infiltrating T Cells and Reduces Regulatory T and Myeloid Cells within B16 Melanoma Tumors,” Proc. Natl. Acad. Sci. U.S.A. 107:4275-4280 (2010); Fife et al., “Control of Peripheral T-cell Tolerance and Autoimmunity Via the CTLA-4 and PD-1 Pathways,” Immunol. Rev. 224:166-182 (2008), each of which is hereby incorporated by reference in its entirety).

Limitations exist in this study. For example, although the eMSCs significantly improved islet survival in allogeneic diabetic mice without any immunosuppression, long-term engraftment in many of the recipients was not achieved. The different outcomes among the recipients might be caused mainly by the lack of MSC persistence in vivo, which resulted in the graft failure and eventual allograft rejection. Previous studies also observed insufficient survival of MSCs at the site of administration, which might be attributed to multiple issues such as apoptosis, hypoxia, and inflammation (Levy et al., “Shattering Barriers Toward Clinically Meaningful MSC Therapies,” Sci. Adv. 6:eaba6884 (2020); Eggenhofer et al., “The Life and Fate of Mesenchymal Stem Cells,” Front. Immunol. 5:148 (2014); Eggenhofer et al., “Mesenchymal Stem Cells Are Short-lived and Do Not Migrate Beyond the Lungs After Intravenous Infusion,” Front. Immunol. 3:297 (2012); Preda et al., “Short Life Span of Syngeneic Transplanted MSC Is a Consequence of in vivo Apoptosis and Immune Cell Recruitment in Mice,” Cell Death Dis. 12:566 (2021), each of which is hereby incorporated by reference in its entirety). Bioengineering strategies such as priming MSCs with hypoxia, inflammatory cytokines and small molecules have been investigated and shown to improve the survival of MSCs (Levy et al., “Shattering Barriers Toward Clinically Meaningful MSC Therapies,” Sci. Adv. 6:eaba6884 (2020); Li et al., “How to Improve the Survival of Transplanted Mesenchymal Stem Cell in Ischemic Heart?,” Stem Cells Int. 2016:9682757 (2016); Hu et al., “Transplantation of Hypoxia-preconditioned Mesenchymal Stem Cells Improves Infarcted Heart Function Via Enhanced Survival of Implanted Cells and Angiogenesis,” J. Thorac. Cardiovasc. Surg. 135:799-808 (2008), each of which is hereby incorporated by reference in its entirety). Together, it was highlighted that the survival of MSCs following local administration needs to be enhanced to improve the therapeutic outcome. There are other factors causing the variation including intrinsic biological variation and unintentional inconsistencies such as cell numbers, spatial distributions, and surgeries. For example, it was challenging to achieve homogeneous dispersion of MSCs and islets when they were delivered to the mouse kidney capsule. While T_{reg} cells surrounding the allograft could modulate the immune responses, some islets could still be exposed to the T_{eff} cells and the gradual T cell infiltration in the long-term would eventually result in destruction of the islets. Better preparation and surgical techniques may improve the therapeutic outcome. Another limitation is that the acquisition and modification of autologous MSCs would be time-consuming, compared to other strategies using off-the-shelf products such as synthetic biomaterial platforms or universal stem cell-derived p cells. Further investigation will be needed to test whether allogeneic modified MSCs have the potential to achieve the same therapeutic effects as autologous modified MSCs. Last, all the experiments described in this study were performed in mice using kidney capsule transplantation without considering autoimmune responses. Although the function of eMSCs in protecting allogeneic islets was not investigated in NOD mice in this study, we showed decreased proliferation, activation, and cytotoxicity of diabetogenic T cells in the in vitro T cell proliferation and activation studies that were carried out using splenocytes isolated from transgenic NOD mice, which might guide the in vivo studies in the future. In addition, future studies should be directed at portal vein transplantation to test whether the eMSCs may be used in clinical islet transplantation to improve graft function and therapeutic outcome with reduced and minimal systemic immunosuppression.

### Example 6—Production of CD47, CD39, CD73, IDO1, and IL-10-Overexpressing MSCs

CD47, CD39, CD73, IDO1 and IL-10 were selected as immunomodulatory proteins for the preparation of eMSCs, where CD47, CD39, and CD73 are cell-surface bound and IL-10 and IDO1 are secreted. To engineer CD47-SIRPα signaling pathway, adenosine pathway, ISO pathway, and IL-10 into MSCs, murine CD47, CD39, CD73, IDO1, and IL-10 genes will be cloned into a lentiviral vector. Human cytomegalovirus immediate early enhancer/promoter (CMV) or human elongation factor-1 alpha (EF1α) will be used to drive their expression, and self-cleaving 2A peptides (T2A, P2A, and F2A) will be placed between each open reading frame to enable polycistronic expression.

The sequence-verified lentiviral vector will be co-transfected with packaging plasmid and envelop plasmid into HEK293T cells, and the lentiviral supernatant will be collected and used to transfect C57BL/6 MSCs. After antibiotic (blasticidin) selection, at least 10 clones will be characterized for selection of those that show optimal expression of these genes.

To confirm expression of CD47, CD39, CD73, and IL-10 in engineered MSCs, Western blot with the respective antibody will also be performed. FACS and ELISA will also be used to further validate that CD47, CD39, and CD73 are expressed on the surface of engineered MSCs, while IL-10 is secreted.

A mixed lymphocyte reaction assay will be used to quantify the immunosuppressive potential of engineered MSCs. 100,000 splenocytes from C57BL/6 mice will be co-cultured with irradiated splenocytes (2000 cGy) from BALB/c mice at a ratio of 1:8. After 24 hours, 20,000 engineered MSCs will be added to the co-culture, and 32 hours later, [3]-thymidine will be added, and uptake of [3]-thymidine will be measured.

Once the immunosuppressive characteristics of the engineered MSCs is demonstrated, the therapeutic potential of the eMSCs as protective accessory cells in islet transplantation will be explored using the procedures of Example 3-5 above.

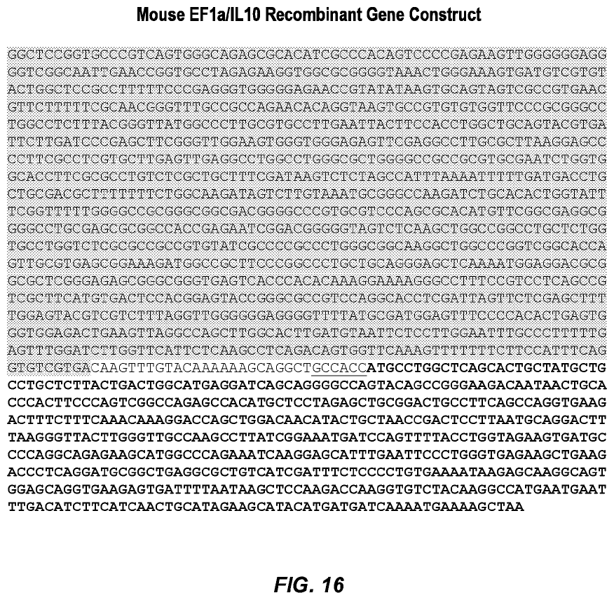

The above co-transfection procedure has been carried with a EF1α/IL-10 transgene illustrated in FIG. 16, and the lentiviral vector was used to transfect the C57BL/6 MSCs. FIGS. 13A-D show that the IL-10 secreting eMSCs are capable of reducing proliferation and activation of CD4⁺ and CD8⁺ T cells, which confirms that these eMSCs can be used to form mixed populations with islet cells and/or islets, and then implanted into patients to treat type 1 diabetes.

### Example 7—Production of Bicistronic Gene Constructs, Lentiviral Vectors, and eMSCs

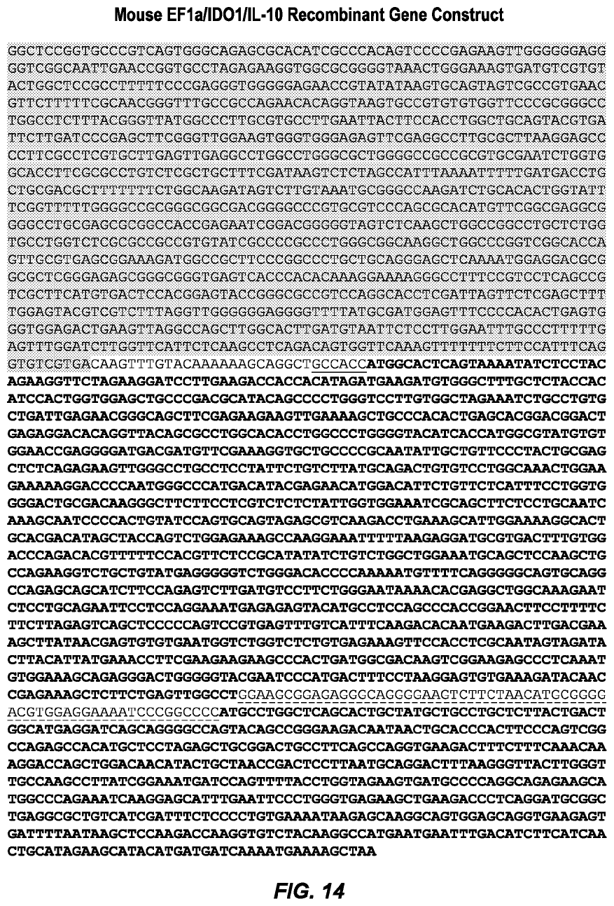

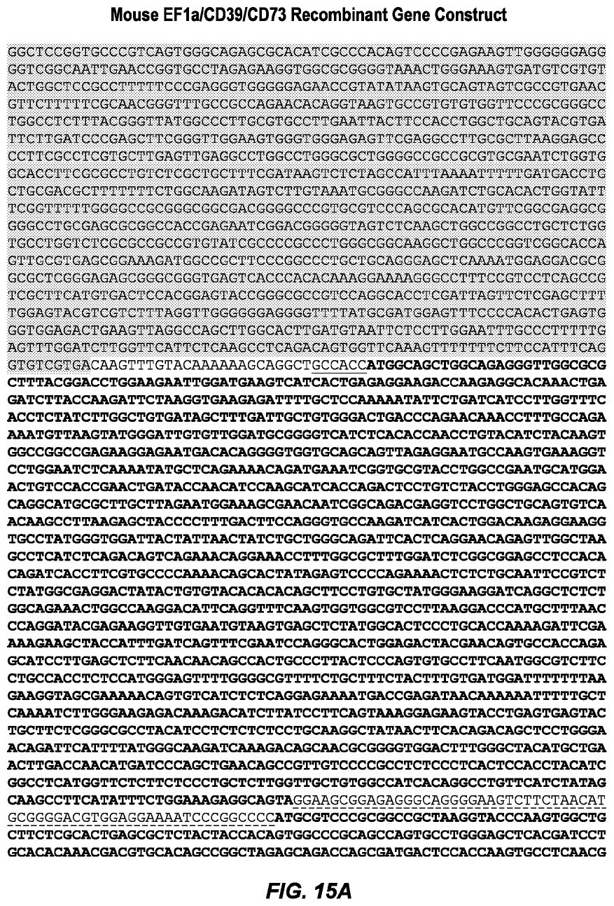

The open reading frames encoding IDO1 and IL10 were synthesized by Integrated DNA Technologies, and then linked together using PCR to generate the bicistronic DNA construct shown in FIG. 14 (SEQ ID NO: 37). This fragment was then inserted into the lentiviral vector using Gibson assembly. After confirming the correct sequence of the lentiviral vector, it was transduced into HEK293T cells using the ViraPower Kit. 2 days after transduction, the supernatant was collected, which was the packaged lentivirus. To generate engineered MSCs, the packaged lentivirus was used to transduce native MSCs as reported in in the Materials & Methods section.

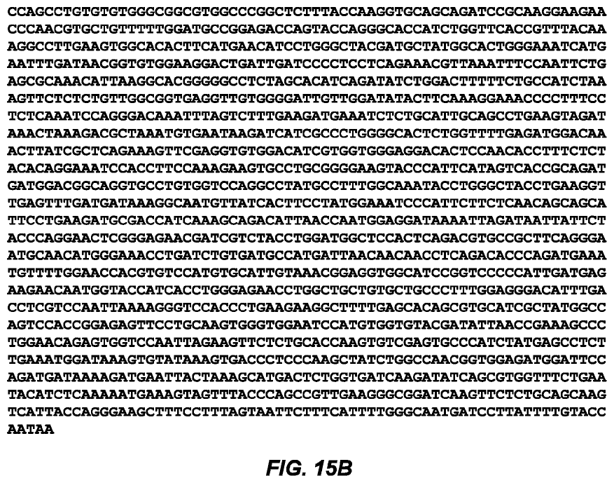

The bicistronic DNA construct containing CD39 and CD73 open reading frames was similarly prepared (see FIGS. 15A-15B, SEQ ID NO: 38), and the lentiviral vector was similarly prepared.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

1. A recombinant mesenchymal stromal cell that expresses one or more immunomodulatory proteins or polypeptides.

2. The recombinant mesenchymal stromal cell according to claim 1, wherein the mesenchymal stromal cell is CD29⁺/SCA-1⁺/CD44⁺ and optionally CD31⁻/CD45⁻/CD117⁻.

3. (canceled)

4. The recombinant mesenchymal stromal cell according to claim 1, wherein one or more immunomodulatory proteins or polypeptides comprises a combination of programmed death ligand-1 (PD-L1) and cytotoxic T lymphocyte antigen 4 immunoglobulin (CTLA4-Ig) fusion protein.

5. The recombinant mesenchymal stromal cell according to claim 4, wherein the mesenchymal stromal cell overexpresses both PD-L1 and CTLA4-Ig fusion protein.

6.-9. (canceled)

10. The recombinant mesenchymal stromal cell according to claim 1, wherein the one or more immunomodulatory proteins or polypeptides comprises one or more of PD-L1, CTLA4-Ig, CD47, CD39, CD73, IL-10, IDO1, Galectin-9, CD155, and Arginase 1.

11. (canceled)

12. The recombinant mesenchymal stromal cell according to claim 1, wherein the recombinant mesenchymal stromal cell comprises either (i) a heterologous transgene comprising a constitutive promoter upstream of a coding sequence of the immunomodulatory protein or polypeptide, or (ii) a constitutive promoter introduced upstream of a coding sequence of a homologous immunomodulatory protein.

13. The recombinant mesenchymal stromal cell according to claim 1, wherein the recombinant mesenchymal stromal cell are human or non-human mammalian mesenchymal stromal cells.

14. (canceled)

15. The recombinant mesenchymal stromal cell according to claim 1, wherein the mesenchymal stromal cells are isolated from a patient sample and then transformed with one or more recombinant expression vectors.

16. (canceled)

17. The recombinant mesenchymal stromal cell according to claim 1, wherein the recombinant mesenchymal stromal cells overexpress one of the immunomodulatory proteins or polypeptides on the cell surface and secretes one of the immunomodulatory proteins or polypeptides.

18. (canceled)

19. A mixed cell population comprising one or more recombinant mesenchymal stromal cells according to claim 1, and one or more cell types distinct of the recombinant mesenchymal stromal cells.

20.-21. (canceled)

22. The mixed cell population according to claim 18, wherein the one or more cell types distinct of the recombinant mesenchymal stromal cells are islet cells.

23. (canceled)

24. The mixed cell population according to claim 22, wherein the islet cells are present in the form of an islet or multiple islets.

25.-27. (canceled)

28. An implantable cell culture device comprising the mixed cell population according to claim 19.

29. The implantable cell culture device according to claim 28, wherein the cell culture device comprises (i) at least one cell culture chamber that contains the mixed cell population and/or (ii) one or more porous coatings that permit passage of soluble factors but inhibit passage of cells.

30. (canceled)

31. A method of improving survival of transplanted cells comprising:
implanting (i) one or more recombinant mesenchymal stromal cells according to claim 1, and (ii) one or more cell types distinct of the recombinant mesenchymal stromal cells into an individual at the same locus, whereby the implanted one or more cell types distinct of the recombinant mesenchymal stromal cells exhibit improved survival compared to said one or more cell types implanted in the absence of the one or more recombinant mesenchymal stromal cells at the same locus.

32. The method according to claim 31, wherein said method is carried out C)_in the absence of administering immunosuppressive agents to the individual, or (ii) using a reduction in the frequency, quantity, or number of immunosuppressive agents administered to the individual.

33. (canceled)

34. The method according to claim 31, wherein the one or more cell types distinct of the recombinant mesenchymal stromal cells are islet cells.

35.-40. (canceled)

41. A method of treating a diabetic subject, said method comprising:
implanting the mixed population of cells according to claim 22 into the diabetic subject, whereby the islet cells express insulin, glucagon, or both to treat the diabetic subject.

42. (canceled)

43. The method according to claim 41, wherein said method is carried out (i) in the absence of administering immunosuppressive agents to the diabetic subject, or (ii) using a reduction in the frequency, quantity, or number of immunosuppressive agents administered to the diabetic subject.

44.-48. (canceled)

49. A method of modifying T cell response to an allograft, the method comprising:
implanting an allograft into an individual with one or more recombinant mesenchymal stromal cells according to claim 1, whereby the one or more recombinant mesenchymal stromal cells cause, relative to an allograft in the absence of the one or more recombinant mesenchymal stromal cells, (i) an increase in the percentage of regulatory T cells (CD4+/CD25+/Foxp3+) present in the implanted graft, and (ii) a reduction in the number of T effector cells (CD4+ or CD8+) present in the implanted graft.

50.-57. (canceled)
